(19) United States
(12) Patent Application Publication
(10) Pub. No.: US 2025/0032604 A1
(43) Pub. Date: Jan. 30, 2025

(54) IMMUNOGENIC COMPOSITIONS AND THEIR USES

(71) Applicant: Flagship Pioneering Innovations VI, LLC, Cambridge, MA (US)

(72) Inventors: Jennifer A. NELSON, Brookline, MA (US); Erik Paul CARTER, Boston, MA (US); Michael Donato MELFI, Somerville, MA (US)

(21) Appl. No.: 18/712,809

(22) PCT Filed: Nov. 23, 2022
(86) PCT No.: PCT/US2022/050908

§ 371 Date: May 23, 2024

Related U.S. Application Data
(60) Provisional application No. 63/283,029, filed on Nov. 24, 2021.

(51) Int. Cl. A61K 39/215; A61K 39/00; A61K 39/145; C07K 14/005; C12N 7/00

(52) U.S. Cl. CPC A61K 39/215; A61K 39/145; C07K 14/005; C12N 7/00; A61K 2039/55516; A61K 2039/575; C07K 2319/735; C12N 2760/16122; C12N 2760/16134; C12N 2770/20021; C12N 2770/20034

(57) ABSTRACT

This disclosure provides compositions, pharmaceutical preparations, and methods relating to circular polyribonucleotides encoding an immunogen and a multimerization domain useful in the development and production of vaccines.

### BACKGROUND

Vaccination has made an enormous contribution to both human and animal health. Since the invention of the first vaccine in 1796, vaccines have come to be considered the most successful method for preventing many infectious diseases by provoking an immune response in a subject. According to the World Health Organization, immunization currently prevents 2-3 million deaths every year across all age groups. Today, vaccines have been developed to prevent and control the spread of more than 20 infectious diseases, including diphtheria, tetanus, pertussis, influenza, and measles, and have led to the complete eradication of smallpox. However, there remains a need to develop new and improved immunogenic compositions and uses thereof.

### SUMMARY

This disclosure provides compositions, pharmaceutical preparations, and methods relating to circular polyribonucleotides encoding one or more immunogens including a multimerization domain. This disclosure also provides methods of using circular polyribonucleotides encoding one or more immunogens including a multimerization domain. This disclosure also provides circular polyribonucleotides including a first expression sequence that encodes an immunogen including a multimerization domain and a second expression sequence that encodes an adjuvant. This disclosure also provides circular polyribonucleotides including an expression sequence that encodes an immunogen including a multimerization domain and a non-coding sequence that stimulates the innate immune system. Compositions and pharmaceutical preparations of circular polyribonucleotides described herein may induce an immune response in a subject upon administration. Compositions and pharmaceutical preparations of circular polyribonucleotides described herein may be used to treat or prevent a disease, disorder, or condition in a subject.

In a first aspect, the disclosure provides a circular polyribonucleotide including an open reading frame including a sequence encoding an immunogen and a sequence encoding a multimerization domain.

In some embodiments, the open reading frame includes, arranged in the following 5′-to-3′ order, a first sequence encoding an immunogen and second sequence encoding a multimerization domain.

In some embodiments, the open reading frame includes, arranged in the following 5′-to-3′ order, a first sequence encoding an immunogen, a second sequence encoding a multimerization domain, and a third sequence encoding an immunogen.

In some embodiments, the open reading frame includes, arranged in the following 5′-to-3′ order, a first sequence encoding an immunogen, a second sequence encoding a multimerization domain, a third sequence encoding an immunogen, and a fourth sequence encoding a multimerization domain.

In some embodiments, the open reading frame includes, arranged in the following 5′-to-3′ order, a first sequence encoding an immunogen, a second sequence encoding a multimerization domain, and a third sequence encoding a multimerization domain.

In some embodiments, the open reading frame includes, arranged in the following 5′-to-3′ order, a first sequence encoding a multimerization domain and second sequence encoding an immunogen.

In some embodiments, the open reading frame includes, arranged in the following 5′-to-3′ order, a first sequence encoding a multimerization domain, a second sequence encoding an immunogen, and a third sequence encoding a multimerization domain.

In some embodiments, the open reading frame includes, arranged in the following 5′-to-3′ order, a first sequence encoding a multimerization domain, a second sequence encoding an immunogen, a third sequence encoding a multimerization domain, and a fourth sequence encoding an immunogen.

In some embodiments, the open reading frame includes, arranged in the following 5′-to-3′ order, a first sequence encoding a multimerization domain, a second sequence encoding a multimerization domain, and a third sequence encoding an immunogen.

In some embodiments, the multimerization domain or each multimerization domain includes a T4 foldon domain. In some embodiments, the multimerization domain or each multimerization domain includes a ferritin domain. In some embodiments, the multimerization domain or each multimerization domain includes a β-annulus peptide. In some embodiments, the multimerization domain or each multimerization domain includes an AaLS peptide. In some embodiments, the multimerization domain or each multimerization domain includes a lumazine synthase domain.

In some embodiments, the open reading frame includes, arranged in the following 5′-to-3′ order, a first sequence encoding an immunogen and second sequence encoding a T4 foldon domain.

In some embodiments, the open reading frame includes, arranged in the following 5′-to-3′ order, a first sequence encoding an immunogen and second sequence encoding a ferritin domain.

In some embodiments, the open reading frame includes, arranged in the following 5′-to-3′ order, a first sequence encoding an immunogen and second sequence encoding a β-annulus peptide.

In some embodiments, the open reading frame includes, arranged in the following 5′-to-3′ order, a first sequence encoding an immunogen and second sequence encoding an AaLS peptide.

In some embodiments, the open reading frame includes, arranged in the following 5′-to-3′ order, a first sequence encoding an immunogen, a second sequence encoding a T4 foldon domain, and a third sequence encoding an immunogen.

In some embodiments, the open reading frame includes, arranged in the following 5′-to-3′ order, a first sequence encoding an immunogen, a second sequence encoding a T4 foldon domain, and a third sequence encoding a ferritin domain.

In some embodiments, the open reading frame includes, arranged in the following 5′-to-3′ order, a first sequence encoding an immunogen, a second sequence encoding a ferritin domain, and a third sequence encoding a T4 foldon domain.

In some embodiments, each immunogen is, independently, operably linked to a secretion signal sequence.

In some embodiments, the open reading frame is operably linked to an IRES.

In some embodiments, the circular polyribonucleotide further includes a second open reading frame encoding a second polypeptide operably linked to a second IRES.

In some embodiments, the second polypeptide is a polypeptide immunogen. In some embodiments, the second polypeptide is a polypeptide adjuvant. In some embodiments, the polypeptide adjuvant is a cytokine, a chemokine, a costimulatory molecule, an innate immune stimulator, a signaling molecule, a transcriptional activator, a cytokine receptor, a bacterial component, a viral component or a component of the innate immune system.

In some embodiments, the circular polyribonucleotide further includes a non-coding ribonucleic acid sequence that is an innate immune system stimulator. In some embodiments, the innate immune system stimulator is selected from a GU-rich motif, an AU-rich motif, a structured region including dsRNA, or an aptamer.

In another aspect, the disclosure provides an immunogenic composition including a circular polyribonucleotide described herein and a pharmaceutically acceptable excipient. In some embodiments, the composition further includes a second circular polyribonucleotide. In some embodiments, the second circular polyribonucleotide includes an open reading frame encoding an immunogen. In some embodiments, the second circular polyribonucleotide includes an open reading frame encoding a polypeptide adjuvant. In some embodiments, the second circular polyribonucleotide includes a non-coding ribonucleic acid sequence that is an innate immune system stimulator.

In another aspect, the disclosure provides a method of inducing an immune response against an immunogen in a subject, the method including administering to the subject a circular polyribonucleotide or immunogenic composition described herein.

In another aspect, the disclosure provides a method of treating or preventing a disease, condition, or disorder in a subject, the method including administering to the subject a circular polyribonucleotide or immunogenic composition described herein.

In some embodiments, the subject is a human subject.

In some embodiments, the method further includes administering an adjuvant to the subject.

In some embodiments, the method further includes administering a polypeptide immunogen to the subject.

### Definitions

The present disclosure will be described with respect to particular embodiments and with reference to certain figures, but the disclosure is not limited thereto but only by the claims. Terms as set forth hereinafter are generally to be understood in their common sense unless indicated otherwise.

As used herein, the term “adaptive immune response” means either a humoral or cell-mediated immune response. For purposes of the present disclosure, a “humoral immune response” refers to an immune response mediated by antibody molecules, while a “cellular immune response” is one mediated by T-lymphocytes and/or other white blood cells.

As used herein, the term “adjuvant” refers to a composition (e.g., a compound, polypeptide, nucleic acid, or lipid) that increases an immune response, for example, increases a specific immune response against an immunogen. Increasing an immune response includes intensification or broadening the specificity of either or both antibody and cellular immune responses.

As used herein, the term “carrier” means a compound, composition, reagent, or molecule that facilitates the transport or delivery of a composition (e.g., a polyribonucleotide) into a subject, a tissue, or a cell. Non-limiting examples of carriers include carbohydrate carriers (e.g., an anhydride-modified phytoglycogen or glycogen-type material), nanoparticles (e.g., a nanoparticle that encapsulates or is covalently linked binds to the circular polyribonucleotide), liposomes, fusosomes, ex vivo differentiated reticulocytes, exosomes, protein carriers (e.g., a protein covalently linked to the polyribonucleotide), or cationic carriers (e.g., a cationic lipopolymer or transfection reagent).

As used herein, the terms “circRNA,” “circular polyribonucleotide,” “circular RNA,” and “circular polyribonucleotide molecule” are used interchangeably and mean a polyribonucleotide molecule that has a structure having no free ends (i.e., no free 3′ and/or 5′ ends), for example a polyribonucleotide molecule that forms a circular or end-less structure through covalent (e.g., covalently closed) or non-covalent bonds. The circular polyribonucleotide may be a covalently closed polyribonucleotide.

As used herein, the term “circularization efficiency” is a measurement of resultant circular polyribonucleotide versus its non-circular starting material.

The term “diluent” means a vehicle including an inactive solvent in which a composition described herein (e.g., a composition including a circular polyribonucleotide) may be diluted or dissolved. A diluent can be an RNA solubilizing agent, a buffer, an isotonic agent, or a mixture thereof. A diluent can be a liquid diluent or a solid diluent. Non-limiting examples of liquid diluents include water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and 1,3-butanediol. Non-limiting examples of solid diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, or powdered sugar.

As used herein, the terms “disease,” “disorder,” and “condition” each refer to a state of sub-optimal health, for example, a state that is or would typically be diagnosed or treated by a medical professional.

As used herein, the term “epitope” refers to a portion or the whole of an immunogen that is recognized, targeted, or bound by an antibody or T cell receptor. An epitope can be a linear epitope, for example, a contiguous sequence of nucleic acids or amino acids. An epitope can be a conformational epitope, for example, an epitope that contains amino acids that form an epitope in the folded conformation of the protein. A conformational epitope can contain non-contiguous amino acids from a primary amino acid sequence. As another example, a conformational epitope includes nucleic acids that form an epitope in the folded conformation of an immunogenic sequence based on its secondary structure or tertiary structure.

As used herein, the term “expression sequence” is a nucleic acid sequence that encodes a product, (e.g., a peptide or polypeptide (e.g., an immunogen), or a regulatory nucleic acid. An exemplary expression sequence that codes for a peptide or polypeptide can include a plurality of nucleotide triads, each of which can code for an amino acid and is termed as a “codon.”

As used herein, the term “fragment” with respect to a polypeptide or a nucleic acid sequence, (e.g., a polypeptide immunogen or a nucleic acid sequence encoding a polypeptide immunogen), refers to a continuous, less than a whole portion of a sequence of the polypeptide or the nucleic acid. A fragment of a polypeptide immunogen or a nucleic acid sequence encoding a polypeptide immunogen, for instance, refers to continuous, less than a whole fraction (e.g., at least 20%, 30%, 40%, 50%, 60%, 70%, 80%, 90%, 95%, or 99% of the entire length) of the sequence such as a sequence disclosed herein. It is understood that all the present disclosure contemplates fragments (e.g., immunogenic fragments) of all immunogens disclosed herein.

As used herein, the term “GC content” refers to the percentage of guanine (G) and cytosine (C) in a nucleic acid sequence. The formula for calculation of the GC content is (G+C)/(A+G+C+U)×100% (for RNA) or (G+C)/(A+G+C+T)×100% (for DNA). Likewise, the term “uridine content” refers to the percentage of uridine (U) in a nucleic acid sequence. The formula for calculation of the uridine content is U/(A+G+C+U)×100%. Likewise, the term “thymidine content” refers to the percentage of thymidine (T) in a nucleic acid sequence. The formula for calculation of the thymidine content is T/(A+G+C+T)×100%.

As used herein, the term “innate immune system stimulator” refers to a substance that induces an innate immunological response, in part, by inducing expression of one or more genes involved in innate immunity, including, but not limited to, a type I interferon (e.g., IFNα, INFβ, and/or IFNγ), a pro-inflammatory cytokine (e.g., IL-1, IL-12, IL-18, TNF-α, and/or GM-CSF), retinoic-acid inducible gene-I (RIG-1, also known as DDX58), melanoma-differentiation-associated gene 5 (MDA5, also known as IFIH1), 2′-5′ oligoadenylate synthase 1 (OAS 1), OAS-like protein (OASL), and/or protein kinase R (PKR). An innate immune system stimulator may act as an adjuvant, (e.g., when administered in combination with or formulated with a ribonucleotide that encodes an immunogen). An innate immune system stimulator may be a separate molecule entity (e.g., not encoded by or incorporated as a sequence in a polyribonucleotide), for example, STING (e.g., caSTING), TLR3, TLR4, TLR9, TLR7, TLR8, TLR7, RIG-I/DDX58, and MDA-5/IFIH1 or a constitutively active mutant thereof. An innate immune system stimulator may be encoded by (e.g., expressed from) a polyribonucleotide. A polyribonucleotide may alternately or further include a ribonucleotide sequence that acts as an innate immune system stimulator (e.g., GU-rich motif, an AU-rich motif, a structured region including dsRNA, or an aptamer).

As used herein, the term “impurity” is an undesired substance present in a composition (e.g., a pharmaceutical composition as described herein). In some embodiments, an impurity is a process-related impurity. In some embodiments, an impurity is a product-related substance other than the desired product in the final composition (e.g., other than the active drug ingredient, e.g., circular polyribonucleotide, as described herein). As used herein, the term “process-related impurity” is a substance used, present, or generated in the manufacturing of a composition, preparation, or product that is undesired in the final composition, preparation, or product other than the linear polyribonucleotides described herein. In some embodiments, the process-related impurity is an enzyme used in the synthesis or circularization of polyribonucleotides. As used herein, the term “product-related substance” is a substance or byproduct produced during the synthesis of a composition, preparation, or product, or any intermediate thereof. In some embodiments, the product-related substance is deoxyribonucleotide fragments. In some embodiments, the product-related substance is deoxyribonucleotide monomers. In some embodiments, the product-related substance is one or more of: derivatives or fragments of polyribonucleotides described herein (e.g., fragments of 10, 9, 8, 7, 6, 5, or 4 ribonucleic acids, monoribonucleic acids, diribonucleic acids, or triribonucleic acids).

As used herein, the term “immunogen” refers to any molecule or molecular structure that includes one or more epitopes recognized, targeted, or bound by an antibody or a T cell receptor. In particular, an immunogen induces an immune response in a subject (e.g., is immunogenic as defined herein). An immunogen is capable of inducing an immune response in a subject, wherein the immune response refers to a series of molecular, cellular, and organismal events that are induced when an immunogen is encountered by the immune system. The immune response may be humoral and/or cellular immune response. These may include the production of antibodies and the expansion of B- and T-cells. To determine whether an immune response has occurred and to follow its course, the immunized subject can be monitored for the appearance of immune reactants directed at the specific immunogen. Immune responses to most immunogens induce the production of both specific antibodies and specific effector T cells. In some embodiments, the immunogen is foreign to a host. In some embodiments, the immunogen is not foreign to a host. An immunogen may include all or a portion of a polypeptide, a polysaccharide, a polynucleotide, or a lipid. An immunogen may also be a mixed polypeptide, polysaccharide, polynucleotide, and/or lipid. For example, an immunogen may be a polypeptide that has been translationally modified. A “polypeptide immunogen” refers to an immunogen that includes a polypeptide. A polypeptide immunogen may also include one or more post-translational modifications, and/or may form a complex with one or more additional molecules, and/or may adopt a tertiary or quaternary structure, each of which may determine or affect the immunogenicity of the polypeptide.

As used herein, the term “immunogenic” refers to a potential to induce a response to a substance in a particular immune response assay above a pre-determined threshold. The assay can be, e.g., expression of certain inflammatory markers, production of antibodies, or an assay for immunogenicity as described herein. In some embodiments, an immune response may be induced when an immune system of an organism or a certain type of immune cells are exposed to an immunogen.

An immunogenic response may be assessed may evaluating the antibodies in the plasma or serum of a subject using a total antibody assay, a confirmatory test, titration and isotyping of the antibodies, and neutralizing antibody assessment. A total antibody assay measures all the antibodies generated as part of the immune response in the serum or plasma of a subject that has been administered the immunogen. The most commonly used test to detect antibodies is an ELISA (enzyme-linked immunosorbent assay), which detects antibodies in the tested serum that bind to the antibody of interest, including IgM, IgD, IgG, IgA, and IgE. An immunogenic response can be further assessed by a confirmatory assay. Following a total antibody assessment, a confirmatory assay may be used to confirm the results of the total antibody assay. A competition assay may be used to confirm that antibody is specifically binding to target and that the positive finding in the screening assay is not a result of non-specific interactions of the test serum or detection reagent with other materials in the assay.

An immunogenic response can be assessed by isotyping and titration. An isotyping assay may be used to assess only the relevant antibody isotypes. For example, the expected isotypes may be IgM and IgG which may be specifically detected and quantified by isotyping and titration, and then compared to the total antibodies present.

An immunogenic response can be assessed by a neutralizing antibody assay (nAb). A neutralizing antibody assay (nAb) may be used to determine if the antibodies produced in response to the immunogen neutralized the immunogen thereby inhibiting the immunogen from having an effect on the target and leading to abnormal pharmacokinetic behaviors. An nAb assay is often a cell-based assay where the target cells are incubated with the antibody. A variety of cell based nAb assays may be used including but not limited to Cell Proliferation, Viability, Antibody-Dependent Cell-Mediated Cytotoxicity (ADCC), Complement-Dependent Cytotoxicity (CDC), Cytopathic Effect Inhibition (CPE), Apoptosis, Ligand Stimulated Cell Signaling, Enzyme Activity, Reporter Gene Assays, Protein Secretion, Metabolic Activity, Stress and Mitochondrial Function. Detection readouts include Absorbance, Fluorescence, Luminescence, Chemiluminescence, or Flow Cytometry. A ligand-binding assay may also be used to measure the binding affinity of an immunogen and an antibody in vitro to evaluate neutralization efficacy.

Furthermore, induction of a cellular immune response may be assessed by measuring T cell activation in a subject using cellular markers on T cells obtained from the subject. A blood sample, lymph node biopsy, or tissue sample can be collected from a subject and T cells from the sample evaluated for one or more (e.g., 2, 3, 4 or more) activation markers: CD25, CD71, CD26, CD27, CD28, CD30, CD154, CD40L, CD134, CD69, CD62L or CD44. T cell activation can also be assessed using the same methods in an in vivo animal model. This assay can also be performed by adding an immunogen to T cells in vitro (e.g., T cells obtained from a subject, animal model, repository, or commercial source) and measuring the aforementioned markers to evaluate T cell activation. Similar approaches can be used to assess the effect of and on activation of other immune cells, such as eosinophils (markers: CD35, CD11 b, CD66, CD69 and CD81), dendritic cells (makers: IL-8, MHC class II, CD40, CD80, CD83, and CD86), basophils (CD63, CD13, CD4, and CD203c), and neutrophils (CD11 b, CD35, CD66b and CD63). These markers can be assessed using flow cytometry, immunohistochemistry, in situ hybridization, and other assays that allow for measurement of cellular markers. Comparing results from before and after administration of an immunogen can be used to determine its effect.

As used herein, the term “inducing an immune response” refers to initiating, amplifying, or sustaining an immune response by a subject. Inducing an immune response may refer to an adaptive immune response or an innate immune response. The induction of an immune response may be measured as discussed above.

As used herein, the term “linear counterpart” is a polyribonucleotide molecule (and its fragments) having the same or similar nucleotide sequence (e.g., 100%, 95%, 90%, 85%, 80%, 75%, or any percentage therebetween sequence identity) as a circular polyribonucleotide and having two free ends (i.e., the uncircularized version (and its fragments) of the circularized polyribonucleotide). In some embodiments, the linear counterpart (e.g., a pre-circularized version) is a polyribonucleotide molecule (and its fragments) having the same or similar nucleotide sequence (e.g., 100%, 95%, 90%, 85%, 80%, 75%, or any percentage therebetween sequence identity) and same or similar nucleic acid modifications as a circular polyribonucleotide and having two free ends (i.e., the uncircularized version (and its fragments) of the circularized polyribonucleotide). In some embodiments, the linear counterpart is a polyribonucleotide molecule (and its fragments) having the same or similar nucleotide sequence (e.g., 100%, 95%, 90%, 85%, 80%, 75%, or any percentage therebetween sequence identity) and different or no nucleic acid modifications as a circular polyribonucleotide and having two free ends (i.e., the uncircularized version (and its fragments) of the circularized polyribonucleotide). In some embodiments, a fragment of the polyribonucleotide molecule that is the linear counterpart is any portion of linear counterpart polyribonucleotide molecule that is shorter than the linear counterpart polyribonucleotide molecule. In some embodiments, the linear counterpart further includes a 5′ cap. In some embodiments, the linear counterpart further includes a poly adenosine tail. In some embodiments, the linear counterpart further includes a 3′ UTR. In some embodiments, the linear counterpart further includes a 5′ UTR.

As used herein, the terms “linear RNA,” “linear polyribonucleotide,” and “linear polyribonucleotide molecule” are used interchangeably and mean polyribonucleotide molecule having a 5′ and 3′ end. One or both of the 5′ and 3′ ends may be free ends or joined to another moiety. Linear RNA includes RNA that has not undergone circularization (e.g., is pre-circularized) and can be used as a starting material for circularization through, for example, splint ligation, or chemical, enzymatic, ribozyme- or splicing-catalyzed circularization methods.

As used herein, the term “modified ribonucleotide” means a nucleotide with at least one modification to the sugar, the nucleobase, or the internucleoside linkage.

As used herein, the term “multimerization domain” refers to a polypeptide domain that self-assembles to form multimers (e.g., dimers, trimers, tetramers, or oligomers). In particular embodiments, a multimerization domain can be fused to a polypeptide (e.g., a polypeptide immunogen). In such instances, fusion to a multimerization domain results in the formation of a multimeric immunogen complex having more than one immunogen upon expression of the polypeptide including an immunogen covalently attached to a multimerization domain.

As used herein, the term “naked delivery” means a formulation for delivery to a cell without the aid of a carrier and without covalent modification to a moiety that aids in delivery to a cell. A naked delivery formulation is free from any transfection reagents, cationic carriers, carbohydrate carriers, nanoparticle carriers, or protein carriers. For example, naked delivery formulation of a circular polyribonucleotide is a formulation that includes a circular polyribonucleotide without covalent modification and is free from a carrier.

As used herein, the terms “nicked RNA,” “nicked linear polyribonucleotide,” and “nicked linear polyribonucleotide molecule” are used interchangeably and mean a polyribonucleotide molecule having a 5′ and 3′ end that results from nicking or degradation of a circular RNA.

As used herein, the term “non-circular RNA” means total nicked RNA and linear RNA.

The term “pharmaceutical composition” is intended to also disclose that the circular polyribonucleotide included within a pharmaceutical composition can be used for the treatment of the human or animal body by therapy. It is thus meant to be equivalent to “a circular polyribonucleotide for use in therapy”.

The term “polynucleotide” as used herein means a molecule including one or more nucleic acid subunits, or nucleotides, and can be used interchangeably with “nucleic acid” or “oligonucleotide”. A polynucleotide can include one or more nucleotides selected from adenosine (A), cytosine (C), guanine (G), thymine (T) and uracil (U), or variants thereof. A nucleotide can include a nucleoside and at least 1, 2, 3, 4, 5, 6, 7, 8, 9, 10, or more phosphate (PO₃) groups. A nucleotide can include a nucleobase, a five-carbon sugar (either ribose or deoxyribose), and one or more phosphate groups. Ribonucleotides are nucleotides in which the sugar is ribose. Polyribonucleotides or ribonucleic acids, or RNA, can refer to macromolecules that include multiple ribonucleotides that are polymerized via phosphodiester bonds. Deoxyribonucleotides are nucleotides in which the sugar is deoxyribose.

“Polydeoxyribonucleotides,” “deoxyribonucleic acids,” and “DNA” mean macromolecules that include multiple deoxyribonucleotides that are polymerized via phosphodiester bonds. A nucleotide can be a nucleoside monophosphate or a nucleoside polyphosphate. A nucleotide means a deoxyribonucleoside polyphosphate, such as, e.g., a deoxyribonucleoside triphosphate (dNTP), which can be selected from deoxyadenosine triphosphate (dATP), deoxycytidine triphosphate (dCTP), deoxyguanosine triphosphate (dGTP), uridine triphosphate (dUTP) and deoxythymidine triphosphate (dTTP) dNTPs, that include detectable tags, such as luminescent tags or markers (e.g., fluorophores). A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). In some examples, a polynucleotide is deoxyribonucleic acid (DNA), ribonucleic acid (RNA), or derivatives or variants thereof. In some cases, a polynucleotide is a short interfering RNA (siRNA), a microRNA (miRNA), a plasmid DNA (pDNA), a short hairpin RNA (shRNA), small nuclear RNA (snRNA), messenger RNA (mRNA), precursor mRNA (pre-mRNA), antisense RNA (asRNA), to name a few, and encompasses both the nucleotide sequence and any structural embodiments thereof, such as single-stranded, double-stranded, triple-stranded, helical, hairpin, etc. In some cases, a polynucleotide molecule is circular. A polynucleotide can have various lengths. A nucleic acid molecule can have a length of at least about 10 bases, 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 50 kb, or more. A polynucleotide can be isolated from a cell or a tissue. As embodied herein, the polynucleotide sequences may include isolated and purified DNA/RNA molecules, synthetic DNA/RNA molecules, and synthetic DNA/RNA analogs.

Polynucleotides (e.g., polyribonucleotides or polydeoxyribonucleotides) may include one or more nucleotide variants, including nonstandard nucleotide(s), non-natural nucleotide(s), nucleotide analog(s) and/or modified nucleotides. Examples of modified nucleotides include, but are not limited to diaminopurine, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudo-uracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid(v), 5-methyl-2-thiouracil, 3-(3-amino-3-N-2-carboxypropyl) uracil, (acp3)w, 2,6-diaminopurine and the like. In some cases, nucleotides may include modifications in their phosphate moieties, including modifications to a triphosphate moiety. Non-limiting examples of such modifications include phosphate chains of greater length (e.g., a phosphate chain having, 4, 5, 6, 7, 8, 9, 10 or more phosphate moieties) and modifications with thiol moieties (e.g., alpha-thiotriphosphate and beta-thiotriphosphates). Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as amino ally 1-dUTP (aa-dUTP) and aminohexhylacrylamide-dCTP (aha-dCTP) to allow covalent attachment of amine reactive moieties, such as N-hydroxy succinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Such alternative base pairs compatible with natural and mutant polymerases for de novo and/or amplification synthesis are described in Betz K, Malyshev D A, Lavergne T, Welte W, Diederichs K, Dwyer T J, Ordoukhanian P, Romesberg F E, Marx A. NAT. CHEM. BIOL. 2012 July; 8(7):612-4, which is herein incorporated by reference for all purposes.

As used herein, “polypeptide” means a polymer of amino acid residues (natural or unnatural) linked together most often by peptide bonds. The term, as used herein, refers to proteins, polypeptides, and peptides of any size, structure, or function. Polypeptides can include gene products, naturally occurring polypeptides, synthetic polypeptides, homologs, orthologs, paralogs, fragments and other equivalents, variants, and analogs of the foregoing. A polypeptide can be a single molecule or may be a multi-molecular complex such as a dimer, trimer, or tetramer. They can also include single chain or multichain polypeptides such as antibodies or insulin and can be associated or linked. Most commonly disulfide linkages are found in multichain polypeptides. The term polypeptide can also apply to amino acid polymers in which one or more amino acid residues are an artificial chemical analogue of a corresponding naturally occurring amino acid.

As used herein, the term “prevent” means to reduce the likelihood of developing a disease, disorder, or condition, or alternatively, to reduce the severity or frequency of symptoms in a subsequently developed disease or disorder. A therapeutic agent can be administered to a subject who is at increased risk of developing a disease or disorder relative to a member of the general population in order to prevent the development of, or lessen the severity of, the disease or condition. A therapeutic agent can be administered as a prophylactic (e.g., before development of any symptom or manifestation of a disease or disorder).

As used interchangeably herein, the terms “polyA” and “polyA sequence” refer to an untranslated, contiguous region of a nucleic acid molecule of at least 5 nucleotides in length and consisting of adenosine residues. In some embodiments, a polyA sequence is at least 10, at least 15, at least 20, at least 30, at least 40, or at least 50 nucleotides in length. In some embodiments, a polyA sequence is located 3′ to (e.g., downstream of) an open reading frame (e.g., an open reading frame encoding a polypeptide), and the polyA sequence is 3′ to a termination element (e.g., a Stop codon) such that the polyA is not translated. In some embodiments, a polyA sequence is located 3′ to a termination element and a 3′ untranslated region.

As used herein, the term “regulatory element” is a moiety, such as a nucleic acid sequence, that modifies expression of an expression sequence within the circular polyribonucleotide.

As used herein, the term “replication element” is a sequence and/or motif useful for replication or that initiates transcription of the circular polyribonucleotide.

As used herein, the terms “systemic delivery” and “systemic administration” mean a route of administration of pharmaceutical compositions or other substances into the circulatory system (e.g., blood or lymphoid system). Systemic administration can include oral administration, parenteral administration, intranasal administration, sublingual administration, rectal administration, transdermal administration, or any combinations thereof. As used herein, the term “non-systemic delivery” or “non-systemic administration” can refer to any other routes of administration than systemic delivery of pharmaceutical compositions or other substances (e.g., the delivered substances do not enter the circulation systems (e.g., blood and lymphoid system)) of the subject body.

As used herein, the term “sequence identity” is determined by alignment of two peptide or two nucleotide sequences using a global or local alignment algorithm. Sequences may then be referred to as “substantially identical” or “essentially similar” when they (when optimally aligned by for example the programs GAP or BESTFIT using default parameters) share at least a certain minimal percentage of sequence identity. GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is a nwsgapdna.cmp scoring matrix and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or EmbossWin version 2.10.0 (using the program “needle”). Alternatively, or additionally, percent identity may be determined by searching against databases, using algorithms such as FASTA, BLAST, etc. Sequence identity refers to the sequence identity over the entire length of the sequence.

A “signal sequence” refers to a polypeptide sequence (e.g., between 10 and 45 amino acids in length) that is present at the N-terminus of a polypeptide sequence of a nascent protein which targets the polypeptide sequence to the secretory pathway.

As used herein, the terms “treat” and “treating” refer to a therapeutic treatment of a disease or disorder (e.g., an infectious disease, a cancer, a toxicity, or an allergic reaction) in a subject. The effect of treatment can include reversing, alleviating, reducing severity of, curing, inhibiting the progression of, reducing the likelihood of recurrence of the disease or one or more symptoms or manifestations of the disease or disorder, stabilizing (i.e., not worsening) the state of the disease or disorder, and/or preventing the spread of the disease or disorder as compared to the state and/or the condition of the disease or disorder in the absence of the therapeutic treatment.

As used herein, the term “termination element” is a moiety, such as a nucleic acid sequence, that terminates translation of the expression sequence in the circular polyribonucleotide.

As used herein, the term “total ribonucleotide molecules” means the total amount of any ribonucleotide molecules, including linear polyribonucleotide molecules, circular polyribonucleotide molecules, monomeric ribonucleotides, other polyribonucleotide molecules, fragments thereof, and modified variations thereof, as measured by total mass of the ribonucleotide molecules

As used herein, the term “translation efficiency” is a rate or amount of protein or peptide production from a ribonucleotide transcript. In some embodiments, translation efficiency can be expressed as amount of protein or peptide produced per given amount of transcript that codes for the protein or peptide (e.g., in a given period of time, e.g., in a given translation system, e.g., an in vitro translation system like rabbit reticulocyte lysate, or an in vivo translation system like a eukaryotic cell or a prokaryotic cell).

As used herein, the term “translation initiation sequence” is a nucleic acid sequence that initiates translation of an expression sequence in the circular polyribonucleotide.

### BRIEF DESCRIPTION OF THE DRAWINGS

FIG. 1 is a schematic of exemplary polyribonucleotide constructs encoding an immunogen and one or more multimerization domains and exemplary corresponding immunogen complexes.

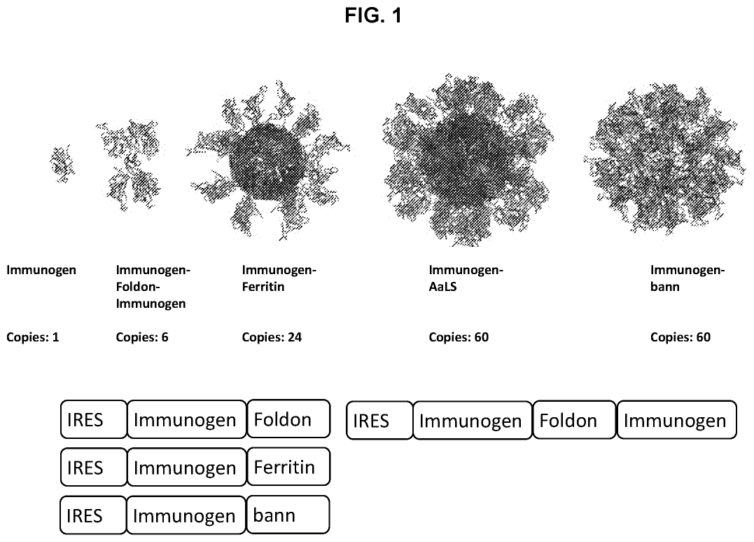

FIG. 2 is a schematic of an exemplary circular RNA that includes two expression sequences, each expression sequence operably connected to an IRES, and where at least one expression sequence is an immunogen including a multimerization domain.

FIG. 3 is a schematic of an exemplary circular RNA that includes two expression sequences, separated by a cleavage domain (e.g., a 2A, a furin site, or a furin-2A), where at least one expression sequence is an immunogen including a multimerization domain, and all are operably linked to an IRES.

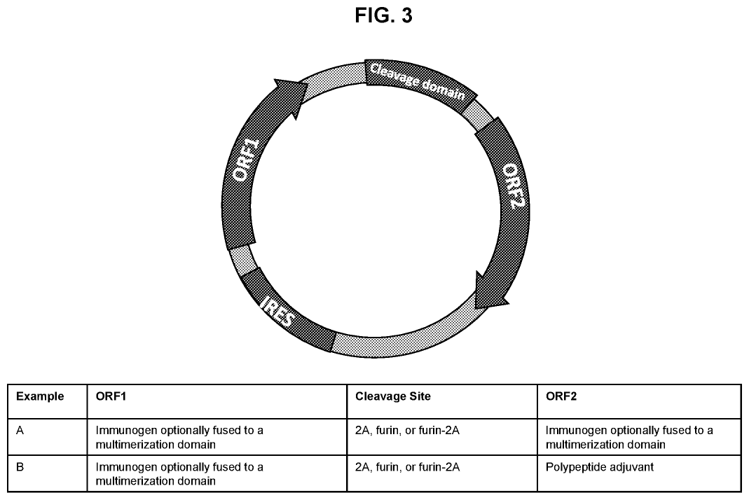

FIG. 4 shows a schematic of a circular RNA that includes an ORF that encodes an immunogen including a multimerization domain and a polynucleotide adjuvant sequence (e.g., a non-coding nucleotide sequence that stimulates the innate immune system).

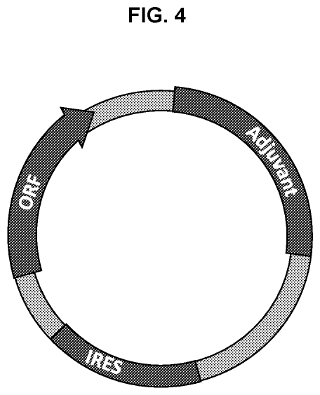

FIG. 5 shows a schematic of a plurality of circular RNAs, where a first circular RNA includes an ORF encoding an immunogen including a multimerization domain and a second circular RNA includes and ORF encoding either a second immunogen or a polypeptide adjuvant.

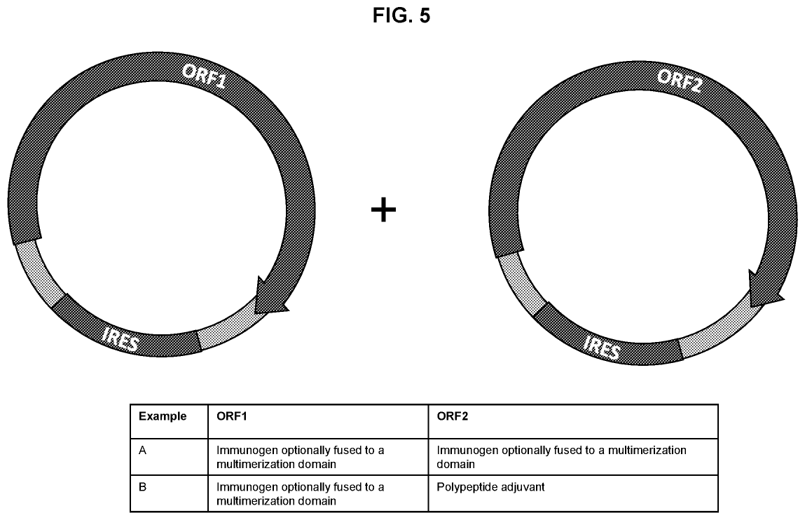

FIG. 6 is a Western blot showing expression of RBD and RBD-Foldon by circular RNA encoding a SARS-CoV-2 RBD immunogen (circRNA-RBD) or circular RNA encoding a SARS-CoV-2 RBD immunogen fused to a Foldon multimerization domain (circRNA-RBD-Foldon) in HEK293T cells at 24 hours after transfection. Asterisk denotes the sample was run under denaturing conditions. FIG. 6 shows that circRNA-RBD expresses RBD monomer and circRNA encoding SARS-CoV-2 RBD immunogen fused to a T4 Foldon multimerization domain expresses and forms trimeric structures in vitro (trimers).

FIG. 7 shows expression of SARS-CoV-2 RBD immunogen in serum of mice after having been administered either a circular RNA encoding a SARS-CoV-2 RBD immunogen or a circular RNA encoding a SARS-CoV-2 RBD immunogen fused to a Foldon multimerization domain.

FIG. 8 shows that binding antibodies were produced in the serum of mice 14 days, 27 days, 35 days, and 42 days after administration of the initial dose (post-prime dose) of either a circular RNA encoding a SARS-CoV-2 RBD immunogen or a circular RNA encoding a SARS-CoV-2 RBD immunogen fused to a foldon multimerization domain.

FIG. 9 shows that T cell responses were primed in mice at 42 days after administration of the initial dose (post-prime dose) of either a circular RNA encoding a SARS-CoV-2 RBD immunogen or a circular RNA encoding a SARS-CoV-2 RBD immunogen fused to a Foldon multimerization domain.

FIG. 10 shows that neutralizing antibodies against SARS-CoV-2 were produced in the serum of mice at 42 days after administration of the initial dose (post-prime dose) of either a circular RNA encoding a SARS-CoV-2 RBD immunogen or a circular RNA encoding a SARS-CoV-2 RBD immunogen fused to a Foldon multimerization domain.

FIG. 11 shows expression of a SARS-CoV-2 RBD immunogen fused to a T4 Foldon multimerization domain in the serum of cynomolgus monkeys after having been administered a 100 μg dose of LNP-formulated circular RNA or a 1000 μg dose of LNP-formulated circular RNA via intramuscular injection.

FIG. 12 shows that RBD-specific antibodies were primed in cynomolgus monkeys at Day 42 after administration of the initial dose (post-prime dose) of either a LNP-formulated circular polyribonucleotide encoding a SARS-CoV-2 RBD immunogen fused to a T4 Foldon multimerization domain or an adjuvanted circular polyribonucleotide encoding a SARS-CoV-2 RBD immunogen fused to a T4 Foldon multimerization domain.

FIG. 13 shows that neutralizing antibodies were primed in cynomolgus monkeys at Day 42 after administration of an initial dose (post-prime dose) of either an LNP-formulated circular polyribonucleotide encoding a SARS-CoV-2 RBD immunogen fused to a T4 Foldon multimerization domain or an adjuvanted circular polyribonucleotide encoding a SARS-CoV-2 RBD immunogen fused to a T4 Foldon multimerization domain.

### DETAILED DESCRIPTION

This disclosure provides compositions, pharmaceutical preparations, and methods relating to circular polyribonucleotides encoding one or more immunogens including a multimerization domain. This disclosure also provides methods of using the circular polyribonucleotides encoding one or more immunogens including a multimerization domain. Compositions and pharmaceutical preparations of circular polyribonucleotides described herein may induce an immune response in a subject upon administration. Compositions and pharmaceutical preparations of circular polyribonucleotides described herein may be used to treat or prevent a disease, disorder, or condition in a subject.

### Circular Polyribonucleotides

The circular polyribonucleotides described herein may include any one or more of the elements described herein and an expression sequence encoding an immunogen including a multimerization domain. In some embodiments, the circular polyribonucleotide includes any feature, or any combination of features as disclosed in International Patent Publication No. WO2019/118919, which is hereby incorporated by reference in its entirety.

In some embodiments, the circular polyribonucleotide is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides.

In some embodiments, the circular polyribonucleotide is between 500 nucleotides and 20,000 nucleotides, between 1,000 and 20,000 nucleotides, between 2,000 and 20,000 nucleotides, or between 5,000 and 20,000 nucleotides. In some embodiments, the circular polyribonucleotide is between 500 nucleotides and 10,000 nucleotides, between 1,000 and 10,000 nucleotides, between 2,000 and 10,000 nucleotides, or between 5,000 and 10,000 nucleotides.

### Immunogens

Circular polyribonucleotides described herein include at least one expression sequence encoding an immunogen including a multimerization domain. Circular polyribonucleotides described herein may include multiple expression sequences, wherein at least one expression sequence encodes an immunogen including a multimerization domain. Circular polyribonucleotides described herein may include two or more (two, three, four, five, six or more) expression sequences, wherein each expression sequence encodes an immunogen including a multimerization domain. Circular polyribonucleotides described herein may include a first expression sequence that encodes an immunogen including a multimerization domain and a second expression sequence that encodes an adjuvant. Circular polyribonucleotides described herein may include an expression sequence that encodes an immunogen including a multimerization domain and a non-coding sequence that stimulates the innate immune system.

An immunogen includes one or more epitopes that are recognized, targeted, or bound by a given antibody or T cell receptor. An epitope can be a linear epitope, for example, a contiguous sequence of nucleic acids or amino acids. An epitope can be a conformational epitope, for example, an epitope that contains amino acids that form an epitope in the folded conformation of the protein. A conformational epitope can contain non-contiguous amino acids from a primary amino acid sequence. As another example, a conformational epitope includes nucleic acids that form an epitope in the folded conformation of an immunogenic sequence based on its secondary structure or tertiary structure.

In some embodiments, an immunogen includes all or a part of a protein, a peptide, a glycoprotein, a lipoprotein, a phosphoprotein, a ribonucleoprotein, a carbohydrate (e.g., a polysaccharide), a lipid (e.g., a phospholipid or triglyceride), or a nucleic acid (e.g., DNA, RNA).

In other embodiments, an immunogen includes a protein immunogen or epitope (e.g., a peptide immunogen or peptide epitope from a protein, glycoprotein, lipoprotein, phosphoprotein, or ribonucleoprotein). An immunogen can include an amino acid, a sugar, a lipid, a phosphoryl, or a sulfonyl group, or a combination thereof.

In a particular embodiment, the immunogen is a polypeptide immunogen.

A polypeptide immunogen may include a post-translational modification, for example, glycosylation, ubiquitination, phosphorylation, nitrosylation, methylation, acetylation, amidation, hydroxylation, sulfation, or lipidation.

In some embodiments, an immunogen includes an epitope including at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids, or more. In some embodiments, an epitope includes or contains at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, or at most 30 amino acids, or less. In some embodiments, an epitope includes or contains 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids. In some embodiments, an epitope contains 5 amino acids. In some embodiments, an epitope contains 6 amino acids. In some embodiments, an epitope contains 7 amino acids. In some embodiments, an epitope contains 8 amino acids. In some embodiments, an epitope can be about 8 to about 11 amino acids. In some embodiments, an epitope can be about 9 to about 22 amino acids.

The immunogens may include immunogens recognized by B cells, immunogens recognized by T cells, or a combination thereof. In some embodiments, the immunogens include immunogens recognized by B cells. In some embodiments, the immunogens are immunogens recognized by B cells. In some embodiments, the immunogens include immunogens recognized by T cells. In some embodiments, the immunogens are immunogens recognized by T cells.

The epitopes may include epitopes recognized by B cells, epitopes recognized by T cells, or a combination thereof. In some embodiments, the epitopes include epitopes recognized by B cells. In some embodiments, the epitopes are epitopes recognized by B cells. In some embodiments, the epitopes include epitopes recognized by T cells. In some embodiments, the epitopes are epitopes recognized by T cells.

Techniques for identifying immunogens and epitopes in silico have been disclosed, for example, in Sanchez-Trincado J L, et al., Fundamentals and methods for T-and B-cell epitope prediction, J. IMMUNOL. RES., 2017:2680160. doi: 10.1155/2017/2680160 (2017)); Grifoni, A, et al., A Sequence Homology and Bioinformatic Approach Can Predict Candidate Targets for Immune Responses to SARS-CoV-2, CELL HOST MICROBE, 27(4):671-80 (2020)); Russi R C et al., In silico prediction of epitopes recognized by T cells and B cells in PmpD: First step towards to the design of a Chlamydia trachomatis vaccine, BIOMEDICAL J., 41(2):109-17 (2018)); Baruah V, et al., Immunoinformatics-aided identification of T cell and B cell epitopes in the surface glycoprotein of 2019-nCoV, J. MED. VIROL., 92(5), doi: 10.1002/jmv.25698 (2020)); each of which is incorporated herein by reference in its entirety.

In some embodiments, an immunogen includes a polynucleotide. In some embodiments, an immunogen is a polynucleotide. In some embodiments, an immunogen includes an RNA. In some embodiments, an immunogen is an RNA. In some embodiments, an immunogen includes a DNA. In some embodiments, an immunogen is a DNA. In some embodiments, the polynucleotide is encoded in the circular or linear polyribonucleotide.

A circular or linear polyribonucleotide of the disclosure includes or encodes any number of immunogens. In a particular embodiment, a circular or linear polyribonucleotide includes or encodes at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, at least 100, at least 120, at least 140, at least 160, at least 180, at least 200, at least 250, at least 300, at least 350, at least 400, at least 450, at least 500, or more of immunogens.

In some embodiments, a circular or linear polyribonucleotide includes or encodes, for example, at most 1, at most 2, at most 3, at most 4, at most 5, at most 6, at most 7, at most 8, at most 9, at most 10, at most 15, at most 20, at most 25, at most 30, at most 40, at most 50, at most 60, at most 70, at most 80, at most 90, at most 100, at most 120, at most 140, at most 160, at most 180, at most 200, at most 250, at most 300, at most 350, at most 400, at most 450, at most 500, or less immunogens.

In some embodiments, a circular or linear polyribonucleotide includes or encodes about 1, 2, 3, 4, 5, 6, 7, 8, 9, 10, 15, 20, 25, 30, 40, 50, 60, 70, 80, 90, 100, 120, 140, 160, 180, 200, 250, 300, 350, 400, 450, or 500 of immunogens.

In some embodiments, the circular or linear polyribonucleotide encodes a plurality of immunogens. In some embodiments, a circular or linear polyribonucleotide includes or encodes between 1 and 100 immunogens. In some embodiments, a circular or linear polyribonucleotide includes or encodes between 1 and 50 immunogens. In some embodiments, a circular or linear polyribonucleotide includes or encodes between 1 and 10 immunogens; for example, a circular or linear polyribonucleotide encodes 1, 2, 3, 4, 5, 6, 7, 8, 9, or 10 immunogens. In some embodiments, a circular or linear polyribonucleotide includes or encodes 2 immunogens. In some embodiments, a circular or linear polyribonucleotide includes or encodes 3 immunogens. In some embodiments, a circular or linear polyribonucleotide includes or encodes 4 immunogens. In some embodiments, a circular or linear polyribonucleotide includes or encodes 5 immunogens.

In some embodiments, the plurality of immunogens each identify the same target. Otherwise put, a single target may include each of the plurality of immunogens, each of the plurality of immunogens may be derived from the same target, and/or each of the plurality of immunogens may share a high degree of similarity with a portion or the whole of the target. For example, a target may be a cell and each of the immunogens may correspond to a protein of that cell. For example, the target may be a particular cancer cell and each of the immunogens may correspond to a tumor antigen associate with that cancer. Accordingly, in some embodiments, each of the plurality of immunogens is derived from different proteins from the same target.

In some embodiments, the immunogens are derived from different targets. In some embodiments, the plurality of immunogens may be derived various capsid proteins of a given virus. For example, the one immunogen may be derived from Orthopoxvirus, another immunogen may be derived Hepadnavirus, and a third immunogen may be derived Flavivirus. For example, a polyribonucleotide may encode multiple immunogens, where each immunogen is derived from yellow fever virus, Chikungunya virus, Zika, Hepatitis A, or Hepatitis B. A polyribonucleotide may encode an immunogen from each of yellow fever virus, Chikungunya virus, Zika, Hepatitis A, and Hepatitis B. A polyribonucleotide may encode multiple immunogens, where each immunogen is derived from Japanese encephalitis, Chikungunya virus, Zika, Hepatitis A, or Hepatitis B. A polyribonucleotide may encode an immunogen from each of Japanese encephalitis, Chikungunya virus, Zika, Hepatitis A, and Hepatitis B. A polyribonucleotide may encode multiple immunogens, where each immunogen is derived from SARS-CoV-2, a poxvirus, respiratory syncytial virus, or human papilloma virus. A polyribonucleotide may encode an immunogen from each of SARS-CoV-2, a poxvirus, respiratory syncytial virus, and human papilloma virus. A polyribonucleotide may encode multiple immunogens, where each immunogen is derived from a herpes virus (CMV, EBV, or VZV). A polyribonucleotide may encode an immunogen from each of the following herpes viruses: CMV, EBV, or VZV. A polyribonucleotide may encode multiple immunogens, where each immunogen is derived Singles or West Nile Virus. A polyribonucleotide may encode an immunogen from each of Shingles and West Nile Virus.

In some embodiments, the immunogens have at least 90%, 91%, 92%, 93%, 94%, 95%, 96%, 97%, 98%, or 99% sequence identity. In some embodiments, the immunogens also have less than 100% sequence identity. This may be indicative of immunogens related to one another by genetic drift, as such, a single circular or linear polyribonucleotide composition or immunogenic composition may be able to induce an immune response against a target that exists in various mutational states in a population or may induce an immune response against multiple targets having the same immunogen where the immunogen is related by genetic drift. For example, the immunogens may be related to one another by genetic drift of a target virus. In some embodiments, the plurality of immunogens may be derived from receptor-binding domains (RBD) from unique but related viruses.

In some embodiments, a circular or linear polyribonucleotide encodes variants of an immunogen. Variants can be naturally occurring variants (for example, variants identified in sequence data from different viral genera, species, isolates, or quasi-species), or can be derivative sequences as disclosed herein that have been generated in silico (for example, immunogens or epitopes with one or more amino acid insertions, deletions, substitutions, or a combination thereof compared to a wild-type immunogen or epitope).

An immunogen is from, for example, a virus, such as a viral surface protein, a viral membrane protein, a viral envelope protein, a viral capsid protein, a viral nucleocapsid protein, a viral spike protein, a viral entry protein, a viral membrane fusion protein, a viral structural protein, a viral non-structural protein, a viral regulatory protein, a viral accessory protein, a secreted viral protein, a viral polymerase protein, a viral DNA polymerase, a viral RNA polymerase, a viral protease, a viral glycoprotein, a viral fusogen, a viral helical capsid protein, a viral icosahedral capsid protein, a viral matrix protein, a viral replicase, a viral transcription factor, or a viral enzyme.

In some embodiments, the immunogen is from one of these viruses:

Orthomyxovirus: Useful immunogens can be from an influenza A, B or C virus, such as the hemagglutinin, neuraminidase, or matrix M2 proteins. Where the immunogen is an influenza A virus hemagglutinin it may be from any subtype (e.g., HI, H2, H3, H4, H5, H6, H7, H8, H9, H10, HII, H12, H13, H14, H15, or H16).

Paramyxoviridae viruses: Viral immunogens include, but are not limited to, those derived from Pneumoviruses (e.g., respiratory syncytial virus (RSV)), Rubulaviruses (e.g., mumps virus), Paramyxoviruses (e.g., parainfluenza virus), Metapneumoviruses and Morbilliviruses (e.g., measles virus), Henipaviruses (e.g., Nipah virus).

Poxviridae: Viral immunogens include, but are not limited to, those derived from Orthopoxvirus such as Variola vera, including but not limited to, Variola major and Variola minor.

Picornavirus: Viral immunogens include, but are not limited to, those derived from Picornaviruses, such as Enteroviruses, Rhinoviruses, Heparnavirus, Cardioviruses and Aphthoviruses. In one embodiment, the enterovirus is a poliovirus (e.g., a type 1, type 2 and/or type 3 poliovirus). In another embodiment, the enterovirus is an EV71 enterovirus. In another embodiment, the enterovirus is a coxsackie A or B virus.

Bunyavirus: Viral immunogens include, but are not limited to, those derived from an Orthobunyavirus, such as California encephalitis virus, a Phlebovirus, such as Rift Valley Fever virus, or a Nairovirus, such as Crimean-Congo hemorrhagic fever virus.

Heparnavirus: Viral immunogens include, but are not limited to, those derived from a Heparnavirus, such as hepatitis A virus (HAV).

Filovirus: Viral immunogens include, but are not limited to, those derived from a filovirus, such as an Ebola virus (including a Zaire, Ivory Coast, Reston, or Sudan ebolavirus) or a Marburg virus.

Togavirus: Viral immunogens include, but are not limited to, those derived from a Togavirus, such as a Rubivirus, an Alphavirus, or an Arterivirus. This includes rubella virus.

Flavivirus: Viral immunogens include, but are not limited to, those derived from a Flavivirus, such as Tick-borne encephalitis (TBE) virus, Dengue (types 1, 2, 3 or 4) virus, Yellow Fever virus, Japanese encephalitis virus, Kyasanur Forest Virus, West Nile encephalitis virus, St. Louis encephalitis virus, Russian spring-summer encephalitis virus, Powassan encephalitis virus, Zika virus.

Pestivirus: Viral immunogens include, but are not limited to, those derived from a Pestivirus, such as Bovine viral diarrhea (BVDV), Classical swine fever (CSFV) or Border disease (BDV).

Hepadnavirus: Viral immunogens include, but are not limited to, those derived from a Hepadnavirus, such as Hepatitis B virus. The hepatitis B virus immunogen may be a hepatitis B virus surface immunogen (HBsAg).

Other hepatitis viruses: Viral immunogens include, but are not limited to, those derived from a hepatitis C virus, delta hepatitis virus, hepatitis E virus, or hepatitis G virus.

Rhabdovirus: Viral immunogens include, but are not limited to, those derived from a Rhabdovirus, such as a Lyssavirus (e.g., a Rabies virus) and Vesiculovirus (VSV).

Caliciviridae: Viral immunogens include, but are not limited to, those derived from Calciviridae, such as Norwalk virus (Norovirus), and Norwalk-like Viruses, such as Hawaii Virus and Snow Mountain Virus.

Retrovirus: Viral immunogens include, but are not limited to, those derived from an Oncovirus, a Lentivirus (e.g., HIV-1 or HIV-2) or a Spumavirus.

Reovirus: Viral immunogens include, but are not limited to, those derived from an Orthoreovirus, a Rotavirus, an Orbivirus, or a Coltivirus.

Parvovirus: Viral immunogens include, but are not limited to, those derived from Parvovirus B119.

Bocavirus: Viral immunogens include, but are not limited to, those derived from bocavirus.

Herpesvirus: Viral immunogens include, but are not limited to, those derived from a human herpesvirus, such as, by way of example only, Herpes Simplex Viruses (HSV) (e.g., HSV types 1 and 2), Varicella-zoster virus (VZV), Epstein-Barr virus (EBV), Cytomegalovirus (CMV), Human Herpesvirus 6 (HHV6), Human Herpesvirus 7 (HHV7), and Human Herpesvirus 8 (HHV8).

Papovaviruses: Viral immunogens include, but are not limited to, those derived from Papillomaviruses and Polyomaviruses. The (human) papillomavirus may be of serotype 1, 2, 4, 5, 6, 8, 11, 13, 16, 18, 31, 33, 35, 39, 41, 42, 47, 51, 57, 58, 63 or 65 (e.g., from one or more of serotypes 6, 11, 16 and/or 18).

Orthohantaviruses: Viral immunogens include, but are not limited to, those derived from hantaviruses.

Arenavirus: Viral immunogens include, but are not limited to, those derived from Guanarito virus, Junin virus, Lassa virus, Lujo virus, Machupo virus, Sabia virus, or Whitewater Arroyo virus.

Adenovirus: Viral immunogens include those derived from adenovirus serotype 36 (Ad-36).

Community acquired respiratory viruses: Viral immunogens include those derived from community acquired respiratory viruses.

Coronavirus: Viral immunogens include, but are not limited to, those derived from a SARS coronavirus (e.g., SARS-CoV-1 and SARS-CoV-2), MERS coronavirus, avian infectious bronchitis (IBV), Mouse hepatitis virus (MHV), and Porcine transmissible gastroenteritis virus (TGEV). The coronavirus immunogen may be a spike polypeptide or a receptor binding domain (RBD) of a spike protein. The coronavirus immunogen may also be an envelope polypeptide, a membrane polypeptide or a nucleocapsid polypeptide.

In some embodiments, the immunogen is from a virus which infects fish. In some embodiments, the immunogen elicits an immune response against a virus which infects fish. For example, the virus which infects fish is selected from infectious salmon anemia virus (ISAV), salmon pancreatic disease virus (SPDV), infectious pancreatic necrosis virus (IPNV), channel catfish virus (CCV), fish lymphocystis disease virus (FLDV), infectious hematopoietic necrosis virus (IHNV), koi herpesvirus, salmon picorna-like virus (also known as picorna-like virus of atlantic salmon), landlocked salmon virus (LSV), atlantic salmon rotavirus (ASR), trout strawberry disease virus (TSD), coho salmon tumor virus (CSTV), or viral hemorrhagic septicemia virus (VHSV).

In some embodiments, an immunogen is from a host subject cell. For example, antibodies that block viral entry can be generated by using an immunogen or epitope from a component of a host cell that a virus uses as an entry factor.

An immunogen is from, for example, a bacteria, such as a bacterial surface protein, a bacterial membrane protein, a bacterial envelope protein, a bacterial inner membrane protein, a bacterial outer membrane protein, a bacterial periplasmic protein, a bacterial entry protein, a bacterial membrane fusion protein, a bacterial structural protein, a bacterial non-structural protein, a secreted bacterial protein, a bacterial polymerase protein, a bacterial DNA polymerase, a bacterial RNA polymerase, a bacterial protease, a bacterial glycoprotein, bacterial transcription factor, a bacterial enzyme, or a bacterial toxin.

In some embodiments, the immunogen elicits an immune response from one of these bacteria: Streptococcus agalactiae (also known as group B streptococcus or GBS)); Streptococcus pyogenes (also known as group A Streptococcus (GAS)); Staphylococcus aureus; Methicillin-resistant Staphylococcus aureus (MRSA); Staphylococcus epidermis; Treponema pallidum; Francisella tularensis; Rickettsia species; Yersinia pestis; Neisseria meningitidis: Immunogens include, but are not limited to, membrane proteins such as adhesins, autotransporters, toxins, iron acquisition proteins, and factor H binding protein; Streptococcus pneumoniae; Moraxella catarrhalis; Bordetella pertussis: Immunogens include, but are not limited to, pertussis toxin or toxoid (PT), filamentous haemagglutinin (FHA), pertactin, and agglutinogens 2 and 3; Clostridium tetani: the typical immunogen is tetanus toxoid; Cornynebacterium diphtheriae: the typical immunogen is diphtheria toxoid; Haemophilus influenzae; Pseudomonas aeruginosa; Chlamydia trachomatis; Chlamydia pneumoniae; Helicobacter pylori; Escherichia coli (Immunogens include, but are not limited to, immunogens derived from enterotoxigenic E. coli (ETEC), enteroaggregative E. coli (EAggEC), diffusely adhering E. coli (DAEC), enteropathogenic E. coli (EPEC), extraintestinal pathogenic E. coli(ExPEC) and/or enterohemorrhagic E. coli(EHEC)). ExPEC strains include uropathogenic E. coli (UPEC) and meningitis/sepsis-associated E. coli (MNEC). Also included are Bacillus anthracis; Clostridium perfringens or Clostridium botulinums; Legionella pneumophila; Coxiella burnetiid; Brucella species, such as B. abortus, B. canis, B. melitensis, B. neotomae, B. ovis, B. suis, and B. pinnipediae. Francisella species, such as F. novicida, F. philomiragia, and F. tularensis; Neisseria gonorrhoeae; Haemophilus ducreyi; Enterococcus faecalis or Enterococcus faecium; Staphylococcus saprophyticus; Yersinia enterocolitica; Mycobacterium tuberculosis; Listeria monocytogenes; Vibrio cholerae; Salmonella typhi; Borrelia burgdorferi; Porphyromonas gingivalis; and Klebsiella species.

An immunogen is from, for example, fungus, such as a fungal surface protein, a fungal membrane protein, a fungal envelope protein, a fungal inner membrane protein, a fungal outer membrane protein, a fungal periplasmic protein, a fungal entry protein, a fungal membrane fusion protein, a fungal structural protein, a fungal non-structural protein, a secreted fungal protein, a fungal polymerase protein, a fungal DNA polymerase, a fungal RNA polymerase, a fungal protease, a fungal glycoprotein, fungal transcription factor, a fungal enzyme, or a fungal toxin.

In some embodiments, the fungal immunogen is derived from Dermatophytes, including: Epidermophyton floccusum, Microsporum audouini, Microsporum canis, Microsporum distortum, Microsporum equinum, Microsporum gypsum, Microsporum nanum, Trichophyton concentricum, Trichophyton equinum, Trichophyton gallinae, Trichophyton gypseum, Trichophyton megnini, Trichophyton mentagrophytes, Trichophyton quinckeanum, Trichophyton rubrum, Trichophyton schoenleini, Trichophyton tonsurans, Trichophyton verrucosum, T. verrucosum var. album, var. discoides, var. ochraceum, Trichophyton violaceum, and/or Trichophyton faviforme; or from Aspergillus fumigatus, Aspergillus flavus, Aspergillus niger, Aspergillus nidulans, Aspergillus terreus, Aspergillus sydowii, Aspergillus flavatus, Aspergillus glaucus, Blastoschizomyces capitatus, Candida albicans, Candida enolase, Candida tropicalis, Candida glabrata, Candida krusei, Candida parapsilosis, Candida stellatoidea, Candida kusei, Candida parakwsei, Candida lusitaniae, Candida pseudotropicalis, Candida guilliermondi, Cladosporium carrionii, Coccidioides immitis, Blastomyces dermatidis, Cryptococcus neoformans, Geotrichum clavatum, Histoplasma capsulatum, Klebsiella pneumoniae, Microsporidia, Encephalitozoon spp., Septata intestinalis and Enterocytozoon bieneusi; the less common are Brachiola spp, Microsporidium spp., Nosema spp., Pleistophora spp., Trachipleistophora spp., Vittaforma spp Paracoccidioides brasiliensis, Pneumocystis carinii, Pythiumn insidiosum, Pityrosporum ovale, Sacharomyces cerevisae, Saccharomyces boulardii, Saccharomyces pombe, Scedosporium apiosperum, Sporothrix schenckii, Trichosporon beigelii, Toxoplasma gondii, Penicillium marneffei, Malassezia spp., Fonsecaea spp., Wangiella spp., Sporothrix spp., Basidiobolus spp., Conidiobolus spp., Rhizopus spp, Mucor spp, Absidia spp, Mortierella spp, Cunninghamella spp, Saksenaea spp., Alternaria spp, Curvularia spp, Helminthosporium spp, Fusarium spp, Aspergillus spp, Penicillium spp, Monolinia spp, Rhizoctonia spp, Paecilomyces spp, Pithomyces spp, and Cladosporium spp.

An immunogen is from, for example, a eukaryotic parasite surface protein, eukaryotic parasite membrane protein, a eukaryotic parasite envelope protein, a eukaryotic parasite entry protein, a eukaryotic parasite membrane fusion protein, a eukaryotic parasite structural protein, a eukaryotic parasite non-structural protein, a secreted eukaryotic parasite protein, a eukaryotic parasite polymerase protein, a eukaryotic parasite DNA polymerase, a eukaryotic parasite RNA polymerase, a eukaryotic parasite protease, a eukaryotic parasite glycoprotein, eukaryotic parasite transcription factor, a eukaryotic parasite enzyme, or a eukaryotic parasite toxin.

In some embodiments, the immunogen elicits an immune response against a parasite from the Plasmodium genus, such as P. falciparum, P. vivax, P. malariae, or P. ovale. In some embodiments, the immunogen elicits an immune response against a parasite from the Caligidae family, particularly those from the Lepeophtheirus and Caligus genera (e.g., sea lice such as Lepeophtheirus salmonis or Caligus rogercresseyi). In some embodiments, the immunogen elicits an immune response against the parasite Toxoplasma gondii.

In some embodiments, the immunogens are cancer immunogens (e.g., neoepitopes). For example, an immunogen is a neoantigen and/or neoepitope that is associated with acute leukemia, astrocytomas, biliary cancer (cholangiocarcinoma), bone cancer, breast cancer, brain stem glioma, bronchioloalveolar cell lung cancer, cancer of the adrenal gland, cancer of the anal region, cancer of the bladder, cancer of the endocrine system, cancer of the esophagus, cancer of the head or neck, cancer of the kidney, cancer of the parathyroid gland, cancer of the penis, cancer of the pleural/peritoneal membranes, cancer of the salivary gland, cancer of the small intestine, cancer of the thyroid gland, cancer of the ureter, cancer of the urethra, carcinoma of the cervix, carcinoma of the endometrium, carcinoma of the fallopian tubes, carcinoma of the renal pelvis, carcinoma of the vagina, carcinoma of the vulva, cervical cancer, chronic leukemia, colon cancer, colorectal cancer, cutaneous melanoma, ependymoma, epidermoid tumors, Ewings sarcoma, gastric cancer, glioblastoma, glioblastoma multiforme, glioma, hematologic malignancies, hepatocellular (liver) carcinoma, hepatoma, Hodgkin's Disease, intraocular melanoma, Kaposi sarcoma, lung cancer, lymphomas, medulloblastoma, melanoma, meningioma, mesothelioma, multiple myeloma, muscle cancer, neoplasms of the central nervous system (CNS), neuronal cancer, small cell lung cancer, non-small cell lung cancer, osteosarcoma, ovarian cancer, pancreatic cancer, pediatric malignancies, pituitary adenoma, prostate cancer, rectal cancer, renal cell carcinoma, sarcoma of soft tissue, schwanoma, skin cancer, spinal axis tumors, squamous cell carcinomas, stomach cancer, synovial sarcoma, testicular cancer, uterine cancer, or tumors and their metastases, including refractory versions of any of the above cancers, or any combination thereof.

In some embodiments, the immunogen is a tumor antigen selected from: (a) cancer-testis antigens such as NY-ESO-1, SSX2, SCP1 as well as RAGE, BAGE, GAGE and MAGE family polypeptides, for example, GAGE-1, GAGE-2, MAGE-1, MAGE-2, MAGE-3, MAGE-4, MAGE-5, MAGE-6, and MAGE-12 (which can be used, for example, to address melanoma, lung, head and neck, NSCLC, breast, gastrointestinal, and bladder tumors; (b) mutated antigens, for example, p53 (associated with various solid tumors, (e.g., colorectal, lung, head and neck cancer), p21/Ras (associated with, e.g., melanoma, pancreatic cancer and colorectal cancer), CDK4 (associated with, e.g., melanoma), MUMI (associated with, e.g., melanoma), caspase-8 (associated with, e.g., head and neck cancer), CIA 0205 (associated with, e.g., bladder cancer), HLA-A2-R1701, beta catenin (associated with, e.g., melanoma), TCR (associated with, e.g., T-cell non-Hodgkins lymphoma), BCR-abl (associated with, e.g., chronic myelogenous leukemia), triosephosphate isomerase, KIA 0205, CDC-27, and LDLR-FUT; (c) over-expressed antigens, for example, Galectin 4 (associated with, e.g., colorectal cancer), Galectin 9 (associated with, e.g., Hodgkin's disease), proteinase 3 (associated with, e.g., chronic myelogenous leukemia), WT 1 (associated with, e.g., various leukemias), carbonic anhydrase (associated with, e.g., renal cancer), aldolase A (associated with, e.g., lung cancer), PRAME (associated with, e.g., melanoma), HER-2/neu (associated with, e.g., breast, colon, lung and ovarian cancer), mammaglobin, alpha-fetoprotein (associated with, e.g., hepatoma), KSA (associated with, e.g., colorectal cancer), gastrin (associated with, e.g., pancreatic and gastric cancer), telomerase catalytic protein, MUC-1 (associated with, e.g., breast and ovarian cancer), G-250 (associated with, e.g., renal cell carcinoma), p53 (associated with, e.g., breast, colon cancer), and carcino embryonic antigen (associated with, e.g., breast cancer, lung cancer, and cancers of the gastrointestinal tract such as colorectal cancer); (d) shared antigens, for example, melanoma-melanocyte differentiation antigens such as MART-I/Melan A, gplOO, MC1R, melanocyte-stimulating hormone receptor, tyrosinase, tyrosinase related protein-1/TRPI and tyrosinase related protein-2/TRP2 (associated with, e.g., melanoma); (e) prostate associated antigens such as PAP, PSA, PSMA, PSH-P1, PSM-P1, PSM-P2, (associated with e.g., prostate cancer); (f) immunoglobulin idiotypes (associated with myeloma and B cell lymphomas, for example); (g) neoantigens. In certain embodiments, tumor immunogens include, but are not limited to, pi 5, Hom/Mel-40, H-Ras, E2A-PRL, H4-RET, IGH-IGK, MYL-RAR, Epstein Barr virus antigens, EBNA, human papillomavirus (HPV) antigens, including E6 and E7, hepatitis B and C virus antigens, human T-cell lymphotropic virus antigens, TSP-180, pl85erbB2, pl80erbB-3, c-met, mn-23H1, TAG-72-4, CA 19-9, CA 72-4, CAM 17.1, NuMa, K-ras, p16, TAGE, PSCA, CT7, 43-9F, 5T4, 791 Tgp72, beta-HCG, BCA225, BTAA, CA 125, CA 15-3 (CA 27.29YBCAA), CA 195, CA 242, CA-50, CAM43, CD68KP1, CO-029, FGF-5, Ga733 (EpCAM), HTgp-175, M344, MA-50, MG7-Ag, MOV18, NB/70K, NY-CO-1, RCAS1, SDCCAG16, TA-90 (Mac-2 binding protein cyclophilin C-associated protein), TAAL6, TAG72, TLP, TPS, and the like.

In some embodiments, the immunogen elicits an immune response against pollen allergens (tree-herb, weed-, and grass pollen allergens); insect or arachnid allergens (inhalant, saliva and venom allergens, e.g., mite allergens, cockroach and midges allergens, hymenopthera venom allergens); animal hair and dandruff allergens (from e.g., dog, cat, horse, rat, mouse, etc.); and food allergens (e.g., a gliadin). Important pollen allergens from trees, grasses and herbs are such originating from the taxonomic orders of Fagales, Oleales, Pinales and platanaceae including, but not limited to, birch (Betula), alder (Alnus), hazel (Corylus), hornbeam (Carpinus) and olive (Olea), cedar (Cryptomeria and Juniperus), plane tree (Platanus), the order of Poales including grasses of the genera Lolium, Phleum, Poa, Cynodon, Dactylis, Holcus, Phalaris, Secale, and Sorghum, the orders of Asterales and Urticales including herbs of the genera Ambrosia, Artemisia, and Parietaria. Other important inhalation allergens are those from house dust mites of the genus Dermatophagoides and Euroglyphus, storage mite (e.g., Lepidoglyphys, Glycyphagus and Tyrophagus), those from cockroaches, midges and fleas (e.g., Blatella, Periplaneta, Chironomus, and Ctenocepphalides), and those from mammals such as cat, dog and horse, venom allergens including such originating from stinging or biting insects such as those from the taxonomic order of Hymenoptera including bees (Apidae), wasps (Vespidea), and ants (Formicoidae).

In some embodiments, the immunogen is derived from, for example, toxin in a venom, such as a venom from a snake (e.g., most species of rattlesnakes (e.g., eastern diamondback rattlesnake), species of brown snakes (e.g., king brown snake and eastern brown snake), russel's viper, cobras (e.g., Indian cobra, king cobra), certain species of kraits (e.g., common krait), mambas (e.g., black mamba), saw-scaled viper, boomslang, dubois sea snake, species of taipans (e.g., coastal taipan and inland taipan snake), species of lanceheads (e.g., fer-de-lance and terciopelo), bushmasters, copperhead, cottonmouth, coral snakes, death adders, Belcher's sea snake, tiger snakes, Australian black snakes), spider (e.g., brown recluse, black widow spider, Brazilian wandering spider, funnel-web spider, button spider, Australian redback spider, katipo, false black widow, Chilean recluse spider, mouse spider, species of Macrothele, species of Sicarius, species of Exophthalmic, certain species of tarantulas), scorpion and other arachnids (e.g., fat-tailed scorpion, deathstalker scorpion, Indian red scorpion, species of Centruroides, species of Tityus such as the Brazilian yellow scorpion), insects (e.g., species of bees, species of wasps, certain ants such as fire ants, some species of lepidopteran caterpillars, certain species of centipede, remipede Xibalbanus tulumensis), fish (e.g., certain species of catfish (e.g., striped eel catfish and other eeltail catfishes), certain species of stingrays (e.g., blue-spotted stingray), lionfishes, stonefishes, scorpionfishes, toadfishes, rabbitfishes, goblinfishes, cockatoo waspfish, striped blenny, stargazers, chimaeras, weevers, dogfish sharks), cnidarians (e.g., certain species jellyfish (e.g., Irukanjdi jellyfish and box jellyfish), hydrozoans (e.g., Portuguese Man o'War), sea anemones, certain species of coral), a lizard (e.g., a gila monster, Mexican bearded lizard, certain species of Varanus (e.g., Komodo dragon), perentie, and lace monitor), a mammal (e.g., Southern short-tailed shrew, duck-billed platypus, European mole, Eurasian water shrew, Mediterranean water shrew, Northern short-tailed shrew, Elliot's short-tailed shrew, certain species of solenodon (e.g., Cuban solenodon, Hispaniolan solenodon), slow loiris), mollusks (e.g., certain species of cone snail), cephalopods (e.g., certain species of octopus (e.g., blue-ringed octopus), squid, and cuttlefish), amphibians (e.g., frogs such as poison dart frogs, Bruno's casque-headed frog, Greening's frog, salamanders (e.g., Fire salamander, Iberian ribbed newt).

In some embodiments, the toxin is from a plant or fungi (e.g., a mushroom).

In some embodiments, the toxin immunogen is derived from a toxin such as a cyanotoxins, dinotoxins, myotoxins, cytotoxins (e.g., ricin, apitoxin, mycotoxins (e.g., aflatoxin), ochratoxin, citrinin, ergot alkaloid, patulin, fusarium, fumonisins, trichothecenes, cardiotoxin), tetrodotoxin, batrachotoxin, botulinum toxin A, tetanus toxin A, diptheria toxin, dioxin, muscarine, bufortoxin, sarin, hemotoxins, phototoxins, necrotoxins, nephrotoxins, and neurotoxins (e.g., calciseptine, cobrotoxin, calcicludine, fasciculin-1, calliotoxin).

Immunogens from any number of microorganisms or cancers can be utilized in the circular or linear polyribonucleotides. In some cases, the immunogens are associated with or expressed by one microorganism disclosed above. In some embodiments, the immunogens are associated with or expressed by two or more microorganisms disclosed above. In some cases, the immunogens are associated with or expressed by one cancer disclosed above. In some embodiments, immunogens are associated with or expressed by two or more cancers disclosed above. In some embodiments, the immunogens are derived from toxins as disclosed above. In some embodiments, the immunogens are from two or more toxins disclosed above.

The two or more microorganisms are related or unrelated. In some cases, two or more microorganisms are phylogenetically related. For example, the circular or linear polyribonucleotides of the disclosure include or encode immunogens from two or more viruses, two or more members of a viral family, two or more members of a viral class, two or more members of a viral order, two or more members of a viral genus, two or more members of a viral species, two or more bacterial pathogens. In some embodiments, the two or more microorganisms are not phylogenetically related.

In some cases, two or more microorganisms are phenotypically related. For example, the circular or linear polyribonucleotides of the disclosure include or encode immunogens from two or more respiratory pathogens, two or more select agents, two or more microorganisms associated with severe disease, two or more microorganisms associated with adverse outcomes in immunocompromised subjects, two or more microorganisms associated with adverse outcomes related to pregnancy, two or more microorganisms associated with hemorrhagic fever.

An immunogen of the disclosure may include a wild-type sequence. When describing an immunogen, the term “wild-type” refers to a sequence (e.g., a nucleic acid sequence or an amino acid sequence) that is naturally occurring and encoded by a genome (e.g., a viral genome). A species (e.g., microorganism species) can have one wild-type sequence, or two or more wild-type sequences (for example, with one canonical wild-type sequence present in a reference microorganism genome, and additional variant wild-type sequences present that have arisen from mutations).

When describing an immunogen, the terms “derivative” and “derived from” refers to a sequence (e.g., nucleic acid sequence or amino acid sequence) that differs from a wild-type sequence by one or more nucleic acids or amino acids, for example, containing one or more nucleic acid or amino acid insertions, deletions, and/or substitutions relative to a wild-type sequence.

An immunogen derivative sequence is a sequence that has at least 60%, 70%, 75%, 80%, 85%, 90%, 91%, 92%, 93%, 94%, 95%, 96%, 97%, 98%, 99%, or more sequence identity to a wild-type sequence, for example, a wild-type nucleic acid, protein, immunogen, or epitope sequence.

In some embodiments, an immunogen contains one or more amino acid insertions, deletions, substitutions, or a combination thereof that affect the structure of an encoded protein. In some embodiments, an immunogen contains one or more amino acid insertions, deletions, substitutions, or a combination thereof that affect the function of an encoded protein. In some embodiments, an immunogen contains one or more amino acid insertions, deletions, substitutions, or a combination thereof that affect the expression or processing of an encoded protein by a cell.

In some embodiments, an immunogen contains one or more nucleic acid insertions, deletions, substitutions, or a combination thereof that affect the structure of an encoded immunogenic nucleic acid.

Amino acid insertions, deletions, substitutions, or a combination thereof can introduce a site for a post-translational modification (for example, introduce a glycosylation, ubiquitination, phosphorylation, nitrosylation, methylation, acetylation, amidation, hydroxylation, sulfation, or lipidation site, or a sequence that is targeted for cleavage). In some embodiments, amino acid insertions, deletions, substitutions, or a combination thereof remove a site for a post-translational modification (for example, remove a glycosylation, ubiquitination, phosphorylation, nitrosylation, methylation, acetylation, amidation, hydroxylation, sulfation, or lipidation site, or a sequence that is targeted for cleavage). In some embodiments, amino acid insertions, deletions, substitutions, or a combination thereof modify a site for a post-translational modification (for example, modify a site to alter the efficiency or characteristics of glycosylation, ubiquitination, phosphorylation, nitrosylation, methylation, acetylation, amidation, hydroxylation, sulfation, or lipidation site, or cleavage).

An amino acid substitution can be a conservative or a non-conservative substitution. A conservative amino acid substitution can be a substitution of one amino acid for another amino acid of similar biochemical properties (e.g., charge, size, and/or hydrophobicity). A non-conservative amino acid substitution can be a substitution of one amino acid for another amino acid with different biochemical properties (e.g., charge, size, and/or hydrophobicity). A conservative amino acid change can be, for example, a substitution that has minimal effect on the secondary or tertiary structure of a polypeptide. A conservative amino acid change can be an amino acid change from one hydrophilic amino acid to another hydrophilic amino acid. Hydrophilic amino acids can include Thr (T), Ser (S), His (H), Glu (E), Asn (N), Gln (Q), Asp (D), Lys (K) and Arg (R). A conservative amino acid change can be an amino acid change from one hydrophobic amino acid to another hydrophilic amino acid. Hydrophobic amino acids can include Ile (I), Phe (F), Val (V), Leu (L), Trp (W), Met (M), Ala (A), Gly (G), Tyr (Y), and Pro (P). A conservative amino acid change can be an amino acid change from one acidic amino acid to another acidic amino acid. Acidic amino acids can include Glu (E) and Asp (D). A conservative amino acid change can be an amino acid change from one basic amino acid to another basic amino acid. Basic amino acids can include His (H), Arg (R) and Lys (K). A conservative amino acid change can be an amino acid change from one polar amino acid to another polar amino acid. Polar amino acids can include Asn (N), Gln (Q), Ser (S) and Thr (T). A conservative amino acid change can be an amino acid change from one nonpolar amino acid to another nonpolar amino acid. Nonpolar amino acids can include Leu (L), Val(V), Ile (I), Met (M), Gly (G) and Ala (A). A conservative amino acid change can be an amino acid change from one aromatic amino acid to another aromatic amino acid. Aromatic amino acids can include Phe (F), Tyr (Y) and Trp (W). A conservative amino acid change can be an amino acid change from one aliphatic amino acid to another aliphatic amino acid. Aliphatic amino acids can include Ala (A), Val (V), Leu (L) and Ile (I). In some embodiments, a conservative amino acid substitution is an amino acid change from one amino acid to another amino acid within one of the following groups: Group 1: Ala, Pro, Gly, Gln, Asn, Ser, Thr; Group II: Cys, Ser, Tyr, Thr; Group III: Val, lie, Leu, Met, Ala, Phe; Group IV: Lys, Arg, His; Group V: Phe, Tyr, Trp, his; and Group VI: Asp, Glu.

In some embodiments, an immunogen derivative or epitope derivative of the disclosure includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, or at least 100 amino acid deletions relative to a sequence disclosed herein (e.g., a wild-type sequence).

In some embodiments, an immunogen derivative or epitope derivative of the disclosure includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid substitutions relative to a sequence disclosed herein (e.g., a wild-type sequence).

In some embodiments, an immunogen derivative or epitope derivative of the disclosure includes at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions relative to a sequence disclosed herein (e.g., a wild-type sequence).

In some embodiments, an immunogen derivative or epitope derivative of the disclosure includes 1-2, 1-3, 1-4, 1-5, 1-6, 1-7, 1-8, 1-9, 1-10, 1-15, 1-20, 1-30, 1-40, 2-3, 2-4, 2-5, 2-6, 2-7, 2-8, 2-9, 2-10, 2-15, 2-20, 2-30, 2-40, 3-3, 3-4, 3-5, 3-6, 3-7, 3-8, 3-9, 3-10, 3-15, 3-20, 3-30, 3-40, 5-6, 5-7, 5-8, 5-9, 5-10, 5-15, 5-20, 5-30, 5-40, 10-15, 15-20, or 20-25 amino acid substitutions relative to a sequence disclosed herein (e.g., a wild-type sequence).

In some embodiments, an immunogen derivative or epitope derivative of the disclosure includes 1, 2, 3, 4, 5, 6, 7, 8, 9, 10, 11, 12, 13, 14, 15, 16, 17, 18, 19, or 20 amino acid substitutions relative to a sequence disclosed herein (e.g., a wild-type sequence).

The one or more amino acid substitutions can be at the N-terminus, the C-terminus, within the amino acid sequence, or a combination thereof. The amino acid substitutions can be contiguous, non-contiguous, or a combination thereof.

In some embodiments, an immunogen derivative or epitope derivative of the disclosure includes at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, at most 50, at most 60, at most 70, at most 80, at most 90, at most 100, at most 120, at most 140, at most 160, at most 180, or at most 200 amino acid deletions relative to a sequence disclosed herein (e.g., a wild-type sequence).

In some embodiments, an immunogen derivative or epitope derivative of the disclosure includes 1-2, 1-3, 1-4, 1-5, 1-6, 1-7, 1-8, 1-9, 1-10, 1-15, 1-20, 1-30, 1-40, 2-3, 2-4, 2-5, 2-6, 2-7, 2-8, 2-9, 2-10, 2-15, 2-20, 2-30, 2-40, 3-3, 3-4, 3-5, 3-6, 3-7, 3-8, 3-9, 3-10, 3-15, 3-20, 3-30, 3-40, 5-6, 5-7, 5-8, 5-9, 5-10, 5-15, 5-20, 5-30, 5-40, 10-15, 15-20, 20-25, 20-30, 30-50, 50-100, or 100-200 amino acid deletions relative to a wild-type sequence.

In some embodiments, an immunogen derivative or epitope derivative of the disclosure includes 1, 2, 3, 4, 5, 6, 7, 8, 9, 10, 11, 12, 13, 14, 15, 16, 17, 18, 19, or 20 amino acid deletions relative to a wild-type sequence.

The one or more amino acid deletions can be at the N-terminus, the C-terminus, within the amino acid sequence, or a combination thereof. The amino acid deletions can be contiguous, non-contiguous, or a combination thereof.

In some embodiments, an immunogen derivative or epitope derivative of the disclosure includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid insertions relative to a wild-type sequence.

In some embodiments, an immunogen derivative or epitope derivative of the disclosure includes at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid insertions relative to a wild-type sequence).

In some embodiments, an immunogen derivative or epitope derivative of the disclosure includes 1-2, 1-3, 1-4, 1-5, 1-6, 1-7, 1-8, 1-9, 1-10, 1-15, 1-20, 1-30, 1-40, 2-3, 2-4, 2-5, 2-6, 2-7, 2-8, 2-9, 2-10, 2-15, 2-20, 2-30, 2-40, 3-3, 3-4, 3-5, 3-6, 3-7, 3-8, 3-9, 3-10, 3-15, 3-20, 3-30, 3-40, 5-6, 5-7, 5-8, 5-9, 5-10, 5-15, 5-20, 5-30, 5-40, 10-15, 15-20, or 20-25 amino acid insertions relative to a wild-type sequence.

In some embodiments, an immunogen derivative or epitope derivative of the disclosure includes 1, 2, 3, 4, 5, 6, 7, 8, 9, 10, 11, 12, 13, 14, 15, 16, 17, 18, 19, or 20 amino acid insertions relative to a wild-type sequence.

The one or more amino acid insertions can be at the N-terminus, the C-terminus, within the amino acid sequence, or a combination thereof. The amino acid insertions can be contiguous, non-contiguous, or a combination thereof.

In some embodiments, the immunogen is expressed by the circular or linear polyribonucleotide.

In some embodiments, the immunogen is a product of rolling circle amplification of the circular or linear polyribonucleotide.

The immunogen may be produced in substantial amounts. As such, the immunogen may be any proteinaceous molecule that can be produced. An immunogen can be a polypeptide that can be secreted from a cell, or localized to the cytoplasm, nucleus, or membrane compartment of a cell. In some embodiments, a polypeptide encoded by a circular or linear polyribonucleotide of the disclosure includes a fusion protein including two or more immunogens disclosed herein. In some embodiments, a polypeptide encoded by a circular or linear polyribonucleotide of the disclosure includes an epitope. In some embodiments, a polypeptide encoded by a circular or linear polyribonucleotide of the disclosure includes a fusion protein including two or more epitopes disclosed herein, for example, an artificial peptide sequence including a plurality of predicted epitopes from one or more microorganisms of the disclosure.

In some embodiments, an immunogen that can be expressed from the circular or linear polyribonucleotide is a membrane protein, for example, including a polypeptide sequence that is generally found as a membrane protein, or a polypeptide sequence that is modified to be a membrane protein. In some embodiments, exemplary immunogens that can be expressed from the circular or linear polyribonucleotide disclosed herein include an intracellular immunogen or cytosolic immunogen.

In some embodiments, the immunogen has a length of less than about 40,000 amino acids, less than about 35,000 amino acids, less than about 30,000 amino acids, less than about 25,000 amino acids, less than about 20,000 amino acids, less than about 15,000 amino acids, less than about 10,000 amino acids, less than about 9,000 amino acids, less than about 8,000 amino acids, less than about 7,000 amino acids, less than about 6,000 amino acids, less than about 5,000 amino acids, less than about 4,000 amino acids, less than about 3,000 amino acids, less than about 2,500 amino acids, less than about 2,000 amino acids, less than about 1,500 amino acids, less than about 1,000 amino acids, less than about 900 amino acids, less than about 800 amino acids, less than about 700 amino acids, less than about 600 amino acids, less than about 500 amino acids, less than about 400 amino acids, less than about 300 amino acids, less than about 250 amino acids, less than about 200 amino acids, less than about 150 amino acids, less than about 140 amino acids, less than about 130 amino acids, less than about 120 amino acids, less than about 110 amino acids, less than about 100 amino acids, less than about 90 amino acids, less than about 80 amino acids, less than about 70 amino acids, less than about 60 amino acids, less than about 50 amino acids, less than about 40 amino acids, less than about 30 amino acids, less than about 25 amino acids, less than about 20 amino acids, less than about 15 amino acids, less than about 10 amino acids, less than about 5 amino acids, any amino acid length therebetween or less may be useful.

In some embodiments, the circular or linear polyribonucleotide includes one or more immunogen sequences and is configured for persistent expression in a cell of a subject in vivo. In some embodiments, the circular or linear polyribonucleotide is configured such that expression of the one or more expression sequences in the cell at a later time point is equal to or higher than an earlier time point.

In such embodiments, the expression of the one or more immunogen sequences can be either maintained at a relatively stable level or can increase over time. The expression of the immunogen sequences can be relatively stable for an extended period of time. The expression of the immunogen sequences can be relatively stable transiently or for only a limited amount of time, for example, at most 1, 2, 3, 4, 5, 6, 7, 8, 9, or 10 days.

In some embodiments, the circular or linear polyribonucleotide expresses one or more immunogens in a subject, (e.g., transiently or long term). In certain embodiments, expression of the immunogens persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In certain embodiments, expression of the immunogens persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or any time therebetween.

The immunogen expression includes translating at least a region of the circular or linear polyribonucleotide provided herein. For example, a circular or linear polyribonucleotide can be translated in a subject to generate polypeptides that include one or more immunogens of the disclosure, thereby stimulating production of an adaptive immune response (e.g., antibody response and/or T cell response) in the subject. In some embodiments, a circular or linear polyribonucleotide of the disclosure is translated to produce one or more immunogens in a human or animal subject, thereby stimulating production of an adaptive immune response (e.g., antibody response and/or T cell response) in a human or animal subject.

In some embodiments, the methods for immunogen expression includes translation of at least 10%, at least 20%, at least 30%, at least 40%, at least 50%, at least 60%, at least 70%, at least 80%, at least 90%, or at least 95% of the total length of the circular or linear polyribonucleotide into polypeptides.

In some embodiments, the methods for immunogen expression includes translation of the circular or linear polyribonucleotide into polypeptides of at least 5 amino acids, at least 10 amino acids, at least 15 amino acids, at least 20 amino acids, at least 50 amino acids, at least 100 amino acids, at least 150 amino acids, at least 200 amino acids, at least 250 amino acids, at least 300 amino acids, at least 400 amino acids, at least 500 amino acids, at least 600 amino acids, at least 700 amino acids, at least 800 amino acids, at least 900 amino acids, or at least 1000 amino acids. In some embodiments, the methods for protein expression includes translation of the circular or linear polyribonucleotide into polypeptides of about 5 amino acids, about 10 amino acids, about 15 amino acids, about 20 amino acids, about 50 amino acids, about 100 amino acids, about 150 amino acids, about 200 amino acids, about 250 amino acids, about 300 amino acids, about 400 amino acids, about 500 amino acids, about 600 amino acids, about 700 amino acids, about 800 amino acids, about 900 amino acids, or about 1000 amino acids. In some embodiments, the methods include translation of the circular or linear polyribonucleotide into continuous polypeptides as provided herein, discrete polypeptides as provided herein, or both.

In some embodiments, the methods for immunogen expression include modification, folding, or other post-translation modification of the translation product. In some embodiments, the methods for immunogen expression include post-translation modification in vivo, (e.g., via cellular machinery).

### Multimerization

In certain embodiments, a circular polyribonucleotide may encode a multimerization domain. For example, a circular polyribonucleotide may encode a first polypeptide that is an immunogen and a second polypeptide that is a multimerization domain. For example, a multimerization domain may be encoded in the same open reading frame as an immunogen and expressed as fusion protein with the immunogen. In some embodiments, the circular polyribonucleotide may encode two or more immunogens, and each immunogen may optionally be fused to a multimerization domain. The multimerization domain may promote the formation of immunogen complexes (e.g., a complex including a plurality of immunogens).

Multimerization of the encoded immunogen may be beneficial for the induction of an immune response. Fusion of the immunogen to one or more multimerization elements (e.g., dimerization elements, trimerization elements, tetramerization elements, and oligomerization elements) may lead to the formation of a multimeric immunogen complex (e.g., formation of a multimeric immunogen complex following expression in an immunized subject). In some embodiments, formation of a multimeric immunogen complex increases immunogenicity of the immunogen. For example, formation of a multimeric immunogen complex may increase immunogenicity of the immunogen by mimicking an infection with an exogenous pathogen (e.g., a virus) where a plurality of potential immunogens is commonly located at the envelope of the pathogen (e.g., hemagglutinin (HA) immunogen of the influenza virus). In some embodiments, the multimerization complex includes at least 2, 3, 4, 6, 8, 10, 15, 20, 25, 30, 35, 40, 45, 50, 55, 60, 70, 80, 90, or 100 immunogens. In some embodiments, the immunogen complex includes 2 to 10, 2 to 50, 2 to 100, 5 to 10, 5 to 15, 5 to 20, 5 to 50, 5 to 100, 10 to 20, 10 to 30, 10 to 40, 10 to 50, 10 to 60, 10 to 100, 20 to 50 or 20 to 100 immunogens. In some embodiments, the immunogen complex includes 6 copies of the immunogen (e.g., the circular polyribonucleotide encodes an immunogen-foldon-immunogen fusion protein). In some embodiments, the immunogen complex includes 24 copies of the immunogen (e.g., the circular polyribonucleotide encodes an immunogen-ferritin fusion protein). In some embodiments, the immunogen complex includes 60 copies of the immunogen (e.g., the circular polyribonucleotide encodes an immunogen-AaLS fusion protein or encodes immunogen-β-annulus peptide).

When used in combination with a polypeptide immunogen of interest in the context of the present disclosure, such multimerization elements can be placed N-terminal or C-terminal to the polypeptide of interest. On nucleic acid level, the coding sequence for such multimerization element is typically placed in the same reading frame, 5′ or 3′ to the coding sequence for the polypeptide or protein of interest.

The multimerization domain may have between 10 and 500 amino acid residues (e.g., between 10 and 450, 10 and 400, 10 and 350, 10 and 300, 10 and 250, 10 and 200, 10 and 150, 10 and 100, 10 and 50, 50 and 500, 100 and 500, 150 and 500, 200 and 500, 250 and 500, 300 and 500, 350 and 500, 400 and 500, and 450 and 500 residues). In some embodiments, the multimerization domain may include between 20 and 2500 amino acid residues (e.g., between 20 and 250, 20 and 225, 20 and 200, 20 and 175, 20 and 150, 20 and 150, 20 and 125, 20 and 100, 20 and 75, 20 and 50, 50 and 250, 75 and 250, 100 and 250, 125 and 250, 150 and 250, 175 and 250, 200 and 250, and 225 and 250 residues).

In some embodiments, an immunogen fused to the multimerization domain is at least 2-fold, 5-fold, or 10-fold more immunogenic than the immunogen (e.g., in a human subject). In some embodiments, the immunogen fused to a multimerization domain is at least 20%, 30%, 40%, 50%, 60%, 70%, 80%, 90%, 100%, 200%, 300%, 400%, or 500% more immunogenic (e.g., in a human subject) than the immunogen not fused to the multimerization domain.

Particular multimerization elements are oligomerization elements, tetramerization elements, trimerization elements or dimerization elements. Dimerization elements may be selected from e.g., dimerization elements/domains of heat shock proteins, immunoglobulin Fc domains and leucine zippers (dimerization domains of the basic region leucine zipper class of transcription factors). Trimerization and tetramerization elements may be selected from e.g., engineered leucine zippers (engineered a-helical coiled coil peptide that adopt a parallel trimeric state), fibritin foldon domain from enterobacteria phage T4, GCN4pll, CCN4-pLI, and p53. In some embodiments, the circular polyribonucleotide includes a T4 foldon domain. In particular embodiments, the T4 foldon domain has an amino acid sequence that is at least 95% identical to GYIPEAPRDGQAYVRKDGEWVLLSTFL (SEQ ID NO: 29). In some embodiments, the T4 foldon has an amino acid sequence of SEQ ID NO: 29. In some embodiments, the multimerization domain is a β-annulus peptide (see, Matsuura et al. (2010), Angew. CHEM. INT. ED., 49: 9662-9665). In some embodiments, the β-annulus peptide has an amino acid sequence of INHVGGTGGAIMAPVAVTRQLVGS (SEQ ID NO: 30), where the C-terminal Serine residue is optionally present or absent or has an amino acid sequence that is at least 95% identical to SEQ ID NO: 30. In some embodiments, the circular polyribonucleotide includes an AaLS peptide. In particular embodiments, the AaLS peptide has an amino acid sequence that is at least 95% identical to TDILGKYVINYLNKLKKKEDIFKEFLKW (SEQ ID NO: 31). In some embodiments, the AaLS peptide has an amino acid sequence of SEQ ID NO: 31.

Oligomerization elements may be selected from e.g., ferritin, surfactant D, oligomerization domains of phosphoproteins of paramyxoviruses, complement inhibitor C4 binding protein (C4 bp) oligomerization domains, Viral infectivity factor (Vif) oligomerization domain, sterile alpha motif (SAM) domain, and von Willebrand factor type D domain.

Ferritin forms oligomers and is a highly conserved protein found in all animals, bacteria, and plants. Ferritin is a protein that spontaneously forms nanoparticles of 24 identical subunits. Ferritin-immunogen fusion constructs potentially form oligomeric aggregates or “clusters” of immunogens that may enhance the immune response. In some embodiments, the circular polyribonucleotide includes a ferritin domain. In some embodiments, the circular polyribonucleotide includes a ferritin domain having the amino acid sequence of:

(SEQ ID NO: 32)
DIIKLLNEQVNKEMNSSNLYMSMSSWCYTHSLDGAGLFLFDHAAEEYEHA
KKLIVFLNENNVPVQLTSISAPEHKFESLTQIFQKAYEHEQHISESINNI
VDHAIKGKDHATFNFLQWYVSEQHEEEVLFKDILDKIELIGNENHGLYLA
DQYVKGIAKSRKS.

Surfactant D protein (SPD) is a hydrophilic glycoprotein that spontaneously self-assembles to form oligomers. An SPD-immunogen fusion constructs may form oligomeric aggregates or “clusters” of immunogens that may enhance the immune response.

Phosphoprotein of paramyxoviruses (negative sense RNA viruses) functions as a transcriptional transactivator of the viral polymerase. Oligomerization of the phosphoprotein is critical for viral genome replication. A phosphoprotein-immunogen fusion constructs may form oligomeric aggregates or “clusters” of immunogens that may enhance the immune response.

Complement inhibitor C4 binding Protein (C4 bp) may also be used as a fusion partner to generate oligomeric immunogen aggregates. The C-terminal domain of C4 bp (57 amino acid residues in humans and 54 amino acid residues in mice) is both necessary and sufficient for the oligomerization of C4 bp or other polypeptides fused to it. A C4 bp-immunogen fusion constructs may form oligomeric aggregates or “clusters” of immunogens that may enhance the immune response. Viral infectivity factor (Vif) multimerization domain has been shown to form oligomers both in vitro and in vivo. The oligomerization of Vif involves a sequence mapping between residues 151 to 164 in the C-terminal domain and the 161 PPLP 164 motif (for human HIV-1: TPKKIKPPLP (SEQ ID NO: 33)). A Vif-immunogen fusion constructs may form oligomeric aggregates or “clusters” of immunogens that may enhance the immune response.

The sterile alpha motif (SAM) domain is a protein interaction module present in a wide variety of proteins involved in many biological processes. The SAM domain that spreads over around 70 residues is found in diverse eukaryotic organisms. SAM domains have been shown to homo- and hetero-oligomerise, forming multiple self-association oligomeric architectures. A SAM-immunogen fusion constructs may form oligomeric aggregates or “clusters” of immunogens that may enhance the immune response. von Willebrand factor (vWF) contains several type D domains: D1 and D2 are present within the N-terminal propeptide whereas the remaining D domains are required for oligomerization. The vWF domain is found in various plasma proteins: complement factors B, C2, C 3 and CR4; the Integrins (I-domains); collagen types VI, VII, XII and XIV; and other extracellular proteins. A vWF-immunogen fusion constructs may form oligomeric aggregates or “clusters” of immunogens that may enhance the immune response.

In some embodiments, the multimerization domain is a lumazine synthase domain. Lumazine synthase may assemble into a complex including 60 copies of the lumazine synthase domain, where each lumazine synthase domain may be fused to one or more immunogens. In some embodiments, the lumazine synthase domain includes an amino acid sequence of any of SEQ ID NOs: 34-44 and 115 or an amino acid sequence having a least 95% sequence identity with any one of SEQ ID NOs: 34-44 and 115.

SEQ ID NO: 34
MQIYEGKLTAEGLRFGIVASRFNHALVDRLVEGAIDAIVRHGGREEDITL
VRVPGSWEIPVAAGELARKEDIDAVIAIGVLIRGATPHFDYIASEVSKGL
ADLSLELRKPITFGVITADTLEQAIERAGTKHGNKGWEAALSAIEMANLF
KSLR
SEQ ID NO: 35
QIYEGKLTAEGLRFGIVASRFNHALVDRLVEGCIDCIVRHGGREEDITLV
RVPGSWEIPVAAGELARKEDIDAVIAIGVLIRGATPHFDYIASEVSKGLA
NLSLELRKPITFGVITADTLEQAIERAGTKHGNKCWEAALSAIEMANLFK
SLR
SEQ ID NO: 36
QIYEGKLTAEGLRFGIVASRFNHALVDRLVEGAIDAIVRHGGREEDITLV
RVPGSWEIPVAAGELARKENISAVIAIGVLIRGATPHFDYIASEVSKGLA
DLSLELRKPITFGVITADTLEQAIERAGTKHGNKGWEAALSAIEMANLFK
SLR
SEQ ID NO: 37
QIYEGKLTAEGLRFGIVASRFNHALVDRLVEGAIDCIVRHGGREEDITLV
RVPGSWEIPVAAGELARKEDIDAVIAIGVLIRGATPHFDYIASEVSKGLA
DLSLELRKPITFGVITADTLEQAIERAGTKHGNKGWEAALSAIEMANLFK
SLR
SEQ ID NO: 115
MQIYEGKLTAEGLRFGIVASRFNHALVDRLVEGAIDCIVRHGGREEDITL
VRVPGSWEIPVAAGELARKEDIDAVIAIGVLIRGATPHFDYIASEVSKGL
ANLSLELRKPITFGVITADTLEQAIERAGTKHGNKGWEAALSAIEMANLF
KSLR

Lumazine synthase domains are provided with one or more cysteine substitutions to introduce non-native disulfide bond(s) that stabilize the lumazine synthase complex formed from self-assembled subunits. In some embodiments, the non-native disulfide bond(s) are introduced with L121C-K131C, L121CG-K131C, L121 GC-K131C, K7C-R40C, 13C-L50C, 182C-K131CG, E5C-R52C, or E95C-A101C substitutions, or a combination thereof (such as 13C-L50C and 182C-K131 CG; E5C-R52C and 182C-K131 CG; or E95C-A101C and 182C-K131CG). The residues numbering is with reference to the lumazine synthase subunit set forth as SEQ ID NO: 34. Non-limiting examples include:

(L121C-K131C)
SEQ ID NO: 38
QIYEGKLTAEGLRFGIVASRFNHALVDRLVEGAIDAIVRHGGREEDITLV
RVPGSWEIPVAAGELARKENISAVIAIGVLIRGATPHFDYIASEVSKGLA
DLSLELRKPITFGVITADTCEQAIERAGTCHGNKGWEAALSAIEMANLFK
SLR
(L121CG-K131C)
SEQ ID NO: 39
QIYEGKLTAEGLRFGIVASRFNHALVDRLVEGAIDAIVRHGGREEDITLV
RVPGSWEIPVAAGELARKENISAVIAIGVLIRGATPHFDYIASEVSKGLA
DLSLELRKPITFGVITADTCCFEQAIERAGTCHGNKGWEAALSAIEMANL
FKSLR
(L121GC-K131C)
SEQ ID NO: 40
QIYEGKLTAEGLRFGIVASRFNHALVDRLVEGAIDAIVRHGGREEDITLV
RVPGSWEIPVAAGELARKENISAVIAIGVLIRGATPHFDYIASEVSKGLA
DLSLELRKPITFGVITADTCFCEQAIERAGTCHGNKGWEAALSAIEMANL
FKSLR
(K7C-R40C)
SEQ ID NO: 41
QIYEGCLTAEGLRFGIVASRFNHALVDRLVEGAIDAIVCVHGGREEDITL
VRVPGSWEIPVAAGELARKENISAVIAIGVLIRGATPHFDYIASEVSKGL
ADLSLELRKPITFGVITADTLEQAIERAGTKHGNKGWEAALSAIEMANLF
KSLR
(13C-L50C, 182C-K131CG)
SEQ ID NO: 42
QCYEGKLTAEGLRFGIVASRFNHALVDRLVEGAIDCIVRHGGREEDITCV
RVPGSWEIPVAAGELARKEDIDAVIAIGVLCRGATPHFDYIASEVSKGLA
DLSLELRKPITFGVITADTLEQAIERAGTCGHGNKGWEAALSAIEMANLF
KSLR
(E5C-R52C, 182C-K131CG)
SEQ ID NO: 43
QIYCGKLTAEGLRFGIVASRFNHALVDRLVEGAIDCIVRHGGREEDITLV
CVPGSWEIPVAAGELARKEDIDAVIAIGVLCRGATPHFDYIASEVSKGLA
DLSLELRKPITFGVITADTLEQAIERAGTCGHGNKGWEAALSAIEMANLF
KSLR
(E95C-A101C, 182C-K131CG)
SEQ ID NO: 44
QIYEGKLTAEGLRFGIVASRFNHALVDRLVEGAIDCIVRHGGREEDITLV
RVPGSWEIPVAAGELARKEDIDAVIAIGVLCRGATPHFDYIASCVSKGLC
DLSLELRKPITFGVITADTLEQAIERAGTCGHGNKGWEAALSAIEMANLF
KSLR

Various methods of multimerization of polypeptides are described International Publication No. WO2020/061564, page 25, line 1 through page 26, line 20, which is herein incorporated by reference.

In some embodiments, the multimerization domain is a riboflavin synthase domain. For example, the riboflavin synthase domain may have an amino acid sequence having a least 95% sequence identity TDILGKYVINYLNKLKKKEDIFKEFLKW (SEQ ID NO: 116). In some embodiments, the riboflavin synthase domain may have an amino acid sequence of SEQ ID NO: 116.

In some embodiments, the circular polyribonucleotide may include one or more multimerization domains. For example, the circular polyribonucleotide may include 2, 3, 4, 5, 6, 7, 8, 9, or 10 multimerization domains. In some embodiments, the circular polyribonucleotide includes two multimerization domains. Two or more multimerization domains may be adjacent to one another. Alternatively, two or more multimerization domains may be separated by one or more other elements. For example, two multimerization domains may be separated by an immunogen. In particular embodiments, the circular polyribonucleotide may include a ferritin domain and a T4 foldon domain. The ferritin and T4 foldon domain may be linked, (e.g., by a Gly-Ser linker). In some embodiments, the ferritin domain linked to the T4 foldon domain has an amino acid sequence of:

(SEQ ID NO: 45)
PGSGYIPEAPRDGQAYVRKDGEWVLLSTFLSGRSGGDIIKLLNEQVNKEM
NSSNLYMSMSSWCYTHSLDGAGLFLFDHAAEEYEHAKKLIVFLNENNVPV
QLTSISAPEHKFESLTQIFQKAYEHEQHISESINNIVDHAIKGKDHATFN
FLQWYVSEQHEEEVLFKDILDKIELIGNENHGLYLADQYVKGIAKSRKS.

Suitable multimerization domains may be selected, for example, from the list of amino acid sequences according to SEQ ID NOs: 1116-1167 of the international patent application WO2017/081082, or fragments or variants of these sequences.

In some embodiments, a circular polyribonucleotide encodes an open reading frame (e.g., an open reading frame operably linked to an IRES) including the elements as described and arranged in Table 1 or Table 2, below. For the embodiments described in Table 1 or Table 2, each immunogen optionally includes a secretion signal sequence. Where an embodiment of Table 1 or Table 2 includes multiple immunogens, the immunogens may be the same or different (e.g., selected from any of the immunogens described herein). Where an embodiment of Table 1 includes multiple multimerization domains, the multimerization domains may be the same or different (e.g., selected from any of the multimerization domains described herein). In some embodiments, a circular polyribonucleotide includes multiple open reading frames, where each open reading frame is described in Table 1 or Table 2.

TABLE 1
Exemplary construct designs including an
immunogen and a multimerization domain

| Region 1 | Region 2 | Region 3 | Region 4 |
|---|---|---|---|
| Immunogen | MD | — | — |
| Immunogen | MD | Immunogen | — |
| Immunogen | MD | Immunogen | MD |
| Immunogen | MD | MD | — |
| MD | Immunogen | — | — |
| MD | Immunogen | MD | — |
| MD | Immunogen | MD | Immunogen |
| MD | MD | Immunogen | — |

*MD = each independently selected from any multimerization domain described herein

TABLE 2
Exemplary construct designs including an
immunogen and a multimerization domain

| Region 1 | Region 2 | Region 3 | Region 4 |
|---|---|---|---|
| Immunogen | T4 Foldon | — | — |
| Immunogen | Ferritin | — | — |
| Immunogen | β-annulus (bann) | — | — |
| Immunogen | AaLS | — | — |
| Immunogen | T4 Foldon | Immunogen | — |
| Immunogen | T4 Foldon | Ferritin | — |
| Immunogen | Ferritin | T4 Foldon | |

### Internal Ribosome Entry Sites

In some embodiments, a circular polyribonucleotide described herein includes one or more internal ribosome entry site (IRES) elements. In some embodiments, the IRES is operably linked to one or more expression sequences (e.g., each IRES is operably linked to one or more expression sequences, where each expression sequence optionally encodes an immunogen, such as an immunogen including a multimerization domain). In embodiments, the IRES is located between a heterologous promoter and the 5′ end of a coding sequence (e.g., a coding sequence encoding an immunogen including a multimerization domain).

A suitable IRES element to include in a polyribonucleotide includes an RNA sequence capable of engaging a eukaryotic ribosome. In some embodiments, the IRES element is at least about 5 nt, at least about 8 nt, at least about 9 nt, at least about 10 nt, at least about 15 nt, at least about 20 nt, at least about 25 nt, at least about 30 nt, at least about 40 nt, at least about 50 nt, at least about 100 nt, at least about 200 nt, at least about 250 nt, at least about 350 nt, or at least about 500 nt.

In some embodiments, the IRES element is derived from the DNA of an organism including, but not limited to, a virus, a mammal, and a Drosophila. Such viral DNA may be derived from, but is not limited to, picomavirus complementary DNA (cDNA), with encephalomyocarditis virus (EMCV) cDNA and poliovirus cDNA. In one embodiment, Drosophila DNA from which an IRES element is derived includes, but is not limited to, an Antennapedia gene from Drosophila melanogaster.

In some embodiments, the IRES sequence is an IRES sequence of Taura syndrome virus, Triatoma virus, Theiler's encephalomyelitis virus, simian Virus 40, Solenopsis invicta virus 1, Rhopalosiphum padi virus, Reticuloendotheliosis virus, fuman poliovirus 1, Plautia stall intestine virus, Kashmir bee virus, Human rhinovirus 2 (HRV-2), Homalodisca coagulata virus-1, Human Immunodeficiency Virus type 1, Homalodisca coagulata virus-1, Himetobi P virus, Hepatitis C virus, Hepatitis A virus, Hepatitis GB virus, foot and mouth disease virus, Human enterovirus 71, Equine rhinitis virus, Ectropis obliqua picorna-like virus, Encephalomyocarditis virus (EMCV), Drosophila C Virus, Crucifer tobamo virus, Cricket paralysis virus, Bovine viral diarrhea virus 1, Black Queen Cell Virus, Aphid lethal paralysis virus, Avian encephalomyelitis virus (AEV), Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1R, Human BAG-I, Human BCL2, Human BiP, Human c-IAPI, Human c-myc, Human eIF4G, Mouse NDST4L, Human LEF1, Mouse HIF1 alpha, Human n.myc, Mouse Gtx, Human p27kipl, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Salivirus, Cosavirus, Parechovirus, Drosophila hairless, S. cerevisiae TFIID, S. cerevisiae YAP1, Human c-src, Human FGF-1, Simian picomavirus, Turnip crinkle virus, Aichivirus, Crohivirus, Echovirus 11, an aptamer to eIF4G, Coxsackievirus B3 (CVB3) or Coxsackievirus A (CVB1/2). In yet another embodiment, the IRES is an IRES sequence of Coxsackievirus B3 (CVB3). In a further embodiment, the IRES is an IRES sequence of Encephalomyocarditis virus. In a further embodiment, the IRES is an IRES sequence of Theiler's encephalomyelitis virus.

The IRES sequence may have a modified sequence in comparison to the wild-type IRES sequence. In some embodiments, when the last nucleotide of the wild-type IRES is not a cytosine nucleic acid residue, the last nucleotide of the wild-type IRES sequence may be modified such that it is a cytosine residue. For example, the IRES sequence may be a CVB3 IRES sequence wherein the terminal adenosine residue is modified to cytosine residue. In some embodiments, the modified CVB3 IRES may have the nucleic acid sequence of:

(SEQ ID NO: 81)
TTAAAACAGCCTGTGGGTTGATCCCACCCACAGGCCCATTGGGCGCTAGC
ACTCTGGTATCACGGTACCTTTGTGCGCCTGTTTTATACCCCCTCCCCCA
ACTGTAACTTAGAAGTAACACACACCGATCAACAGTCAGCGTGGCACACC
AGCCACGTTTTGATCAAGCACTTCTGTTACCCCGGACTGAGTATCAATAG
ACTGCTCACGCGGTTGAAGGAGAAAGCGTTCGTTATCCGGCCAACTACTT
CGAAAAACCTAGTAACACCGTGGAAGTTGCAGAGTGTTTCGCTCAGCACT
ACCCCAGTGTAGATCAGGTCGATGAGTCACCGCATTCCCCACGGGCGACC
GTGGCGGTGGCTGCGTTGGCGGCCTGCCCATGGGGAAACCCATGGGACGC
TCTAATACAGACATGGTGCGAAGAGTCTATTGAGCTAGTTGGTAGTCCTC
CGGCCCCTGAATGCGGCTAATCCTAACTGCGGAGCACACACCCTCAAGCC
AGAGGGCAGTGTGTCGTAACGGGCAACTCTGCAGCGGAACCGACTACTTT
GGGTGTCCGTGTTTCATTTTATTCCTATACTGGCTGCTTATGGTGACAAT
TGAGAGATCGTTACCATATAGCTATTGGATTGGCCATCCGGTGACTAATA
GAGCTATTATATATCCCTTTGTTGGGTTTATACCACTTAGCTTGAAAGAG
GTTAAAACATTACAATTCATTGTTAAGTTGAATACAGCAAC

In some embodiments, the IRES sequence is an Enterovirus 71 (EV17) IRES. In some embodiments, the terminal guanosine residue of the EV17 IRES sequence is modified to a cytosine residue. In some embodiments, the modified EV71 IRES may have the nucleic acid sequence of:

(SEQ ID NO: 94)
ACGTTACTGGCCGAAGCCGCTTGGAATAAGGCCGGTGTGCGTTTGTCTAT
ATGTTATTTTCCACCATATTGCCGTCTTTTGGCAATGTGAGGGCCCGGAA
ACCTGGCCCTGTCTTCTTGACGAGCATTCCTAGGGGTCTTTCCCCTCTCG
CCAAAGGAATGCAAGGTCTGTTGAATGTCGTGAAGGAAGCAGTTCCTCTG
GAAGCTTCTTGAAGACAAACAACGTCTGTAGCGACCCTTTGCAGGCAGCG
GAACCCCCCACCTGGCGACAGGTGCCTCTGCGGCCAAAAGCCACGTGTAT
AAGATACACCTGCAAAGGCGGCACAACCCCAGTGCCACGTTGTGAGTTGG
ATAGTTGTGGAAAGAGTCAAATGGCTCTCCTCAAGCGTATTCAACAAGGG
GCTGAAGGATGCCCAGAAGGTACCCCATTGTATGGGATCTGATCTGGGGC
CTCGGTGCACATGCTTTACATGTGTTTAGTCGAGGTTAAAAAACGTCTAG
GCCCCCCGAACCACGGGGACGTGGTTTTCCTTTGAAAAACACGATGATAA
TA

In some embodiments, the polyribonucleotide includes at least one IRES flanking at least one (e.g., 2, 3, 4, 5 or more) expression sequence. In some embodiments, the IRES flanks both sides of at least one (e.g., 2, 3, 4, 5 or more) expression sequence. In some embodiments, the polyribonucleotide includes one or more IRES sequences on one or both sides of each expression sequence, leading to separation of the resulting peptide(s) and or polypeptide(s). For example, a polyribonucleotide described herein may include a first IRES operably linked to a first expression sequence (e.g., encoding a first immunogen, such as a first immunogen including a multimerization domain) and a second IRES operably linked to a second expression sequence (e.g., encoding a second immunogen, such as a second immunogen including a multimerization domain).

In some embodiments, a polyribonucleotide described herein includes an IRES (e.g., an IRES operably linked to a coding region). For example, the polyribonucleotide may include any IRES as described in Chen et al. MOL. CELL 81(20):4300-18, 2021; Jopling et al. ONCOGENE 20:2664-70, 2001; Baranick et al. PNAS 105(12):4733-38, 2008; Lang et al. MOLECULAR BIOLOGY OF THE CELL 13(5):1792-1801, 2002; Dorokhov et al. PNAS 99(8):5301-06, 2002; Wang et al. NUCLEIC ACIDS RESEARCH 33(7):2248-58, 2005; Petz et al. NUCLEIC ACIDS RESEARCH 35(8):2473-82, 2007; Chen et al. SCIENCE 268:415-417, 1995; Fan et al. NATURE COMMUNICATION 13(1):3751-3765, 2022, and International Publication No. WO2021/263124, each of which is hereby incorporated by reference in their entirety.

### Signal Sequences

In some embodiments, exemplary immunogens that can be expressed from a circular polyribonucleotide disclosed herein include a secreted protein, for example, a protein (e.g., immunogen) that naturally includes a signal sequence, or one that does not usually encode a signal sequence but is modified to contain one. In some embodiments, the immunogen(s) encoded by the circular polyribonucleotide includes a secretion signal. For example, the secretion signal may be the naturally encoded secretion signal for a secreted protein. In another example, the secretion signal may be a modified secretion signal for a secreted protein. In other embodiments, the immunogen(s) encoded by the circular polyribonucleotide do not include a secretion signal.

In some embodiments, the signal sequence is selected from

| | SecSP38 |
|---|---|
| SEQ ID NO: 1 | |
| | (MWWRLWWLLLLLLLLWPMVWA;); |
| | SecD4 |
| SEQ ID NO: 2 | |
| | (MWWLLLLLLLLWPMVWA;), |
| | gLuc |
| SEQ ID NO: 3 | |
| | (MGVKVLFALICIAVAEAK;); |
| | INHC1 |
| SEQ ID NO: 4 | |
| | (MASRLTLLTLLLLLLAGDRASS;); |
| | Epo |
| SEQ ID NO: 5 | |
| | (MGVHECPAWLWLLLSLLSLPLGLPVLG;); |
| | and |
| | IL-2 |
| SEQ ID NO: 6 | |
| | (MYRMQLLSCIALSLALVTNS;). |

In some embodiments, a circular polyribonucleotide encodes multiple copies of the same immunogen (e.g., one, two, three, four, five, six, seven, eight, nine, ten, or more). In some embodiments, at least one copy of the immunogen includes a signal sequence and at least one copy of the immunogen does not include a signal sequence. In some embodiments, a circular polyribonucleotide encodes plurality of immunogens (e.g., a plurality of different immunogens or a plurality of immunogens having less than 100% sequence identity), where at least one of the plurality of immunogens includes a signal sequence and at least one copy of the plurality of immunogens does not include a signal sequence.

In some embodiments, the signal sequence is a wild-type signal sequence that is present on the N-terminus of the corresponding wild-type immunogen, (e.g., when expressed endogenously). In some embodiments, the signal sequence is heterologous to the immunogen, (e.g., is not present when the wild-type immunogen is expressed endogenously). A polyribonucleotide sequence encoding an immunogen may be modified to remove the nucleotide sequence encoding a wild-type signal sequence and/or add a sequence encoding a heterologous signal sequence.

The circular polyribonucleotide may further include one or more adjuvants, each with or without a signal sequence. In some embodiments, the circular polyribonucleotide encodes at least one adjuvant and at least one immunogen. In some embodiments, the at least one encoded adjuvant includes a signal sequence and the at least one encoded immunogen does not include a signal sequence. In some embodiments, the at least one encoded adjuvant includes a signal sequence and the at least one encoded immunogen includes a signal sequence. In some embodiments, the at least one encoded adjuvant does not include a signal sequence and the at least one encoded immunogen includes a signal sequence. In some embodiments, neither the encoded adjuvant nor the encoded immunogen includes a signal sequence.

In some embodiments, the signal sequence is a wild-type signal sequence that is present on the N-terminus of the corresponding wild-type adjuvant, (e.g., when expressed endogenously). In some embodiments, the signal sequence is heterologous to the adjuvant, (e.g., is not present when the wild-type adjuvant is expressed endogenously). A polyribonucleotide sequence encoding an adjuvant may be modified to remove the nucleotide sequence encoding a wild-type signal sequence and/or add a sequence encoding a heterologous signal sequence.

A polypeptide encoded by a polyribonucleotide (e.g., immunogen or an adjuvant encoded by a polyribonucleotide) may include a signal sequence that directs the immunogen or adjuvant to the secretory pathway. In some embodiments, the signal sequence may direct the immunogen or adjuvant to reside in certain organelles (e.g., the endoplasmic reticulum, Golgi apparatus, or endosomes). In some embodiments, the signal sequence directs the immunogen or adjuvant to be secreted from the cell. For secreted proteins, the signal sequence may be cleaved after secretion, resulting in a mature protein. In other embodiments, the signal sequence may become embedded in the membrane of the cell or certain organelles, creating a transmembrane segment that anchors the protein to the membrane of the cell, endoplasmic reticulum, or Golgi apparatus. In certain embodiments, the signal sequence of a transmembrane protein is a short sequence at the N-terminal of the polypeptide. In other embodiments, the first transmembrane domain acts as the first signal sequence, which targets the protein to the membrane.

In some embodiments, an adjuvant encoded by a polyribonucleotide includes a secretion signal sequence. In some embodiments, an immunogen encoded by a polyribonucleotide includes either a secretion signal sequence, a transmembrane insertion signal sequence, or does not include a signal sequence.

### Regulatory Elements

In some embodiments, a circular polyribonucleotide includes one or more regulatory elements, (e.g., one or more sequences that modify expression of an expression sequence within the circular polyribonucleotide).

A regulatory element may include a sequence that is located adjacent to an expression sequence that encodes an expression product. A regulatory element may be operably linked to the adjacent sequence. A regulatory element may increase an amount of product expressed as compared to an amount of the expressed product when no regulatory element is present. A regulatory element may be used to increase the expression of one or more immunogen(s) and/or adjuvant(s) encoded by a circular polyribonucleotide. Likewise, a regulatory element may be used to decrease the expression of one or more immunogen(s) and/or adjuvant(s) encoded by a circular polyribonucleotide. In some embodiments, a regulatory element may be used to increase expression of an immunogen and/or adjuvant and another regulatory element may be used to decrease expression of another immunogen and/or adjuvant on the same circular polyribonucleotide. In addition, one regulatory element can increase an amount of product (e.g., an immunogen or adjuvants) expressed for multiple expression sequences attached in tandem. Hence, one regulatory element can enhance the expression of one or more expression sequences (e.g., immunogens or adjuvants). Multiple regulatory elements can also be used, for example, to differentially regulate expression of different expression sequences.

In some embodiments, a regulatory element as provided herein can include a selective translation sequence. As used herein, the term “selective translation sequence” refers to a nucleic acid sequence that selectively initiates or activates translation of an expression sequence in the circular polyribonucleotide, for instance, certain riboswitch aptazymes. A regulatory element can also include a selective degradation sequence. As used herein, the term “selective degradation sequence” refers to a nucleic acid sequence that initiates degradation of the circular polyribonucleotide, or an expression product of the circular polyribonucleotide. In some embodiments, the regulatory element is a translation modulator. A translation modulator can modulate translation of the expression sequence in the circular polyribonucleotide. A translation modulator can be a translation enhancer or suppressor. In some embodiments, a translation initiation sequence can function as a regulatory element.

In some embodiments, a circular polyribonucleotide produces stoichiometric ratios of expression products. Rolling circle translation continuously produces expression products at substantially equivalent ratios. In some embodiments, the circular polyribonucleotide has a stoichiometric translation efficiency, such that expression products are produced at substantially equivalent ratios. In some embodiments, the circular polyribonucleotide has a stoichiometric translation efficiency of multiple expression products, (e.g., products from 2, 3, 4, 5, 6, 7, 8, 9, 10, 11, 12, or more expression sequences). In some embodiments, the circular polyribonucleotide produces substantially different ratios of expression products. For example, the translation efficiency of multiple expression products may have a ratio of 1:10,000; 1:7000, 1:5000, 1:1000, 1:700, 1:500, 1:100, 1:50, 1:10, 1:5, 1:4, 1:3 or 1:2. In some embodiments, the ratio of multiple expression products may be modified using a regulatory element.

Further examples of regulatory elements are described in paragraphs [0154]-[0161] of International Patent Publication No. WO2019/118919, which is hereby incorporated by reference in its entirety.

### Cleavage Domains

A circular polyribonucleotide of the disclosure can include a cleavage domain (e.g., a stagger element or a cleavage sequence).

The term “stagger element” refers to a moiety, such as a nucleotide sequence, that induces ribosomal pausing during translation. In some embodiments, the stagger element is a non-conserved sequence of amino-acids with a strong alpha-helical propensity followed by the consensus sequence—D(V/I)ExNPGP, where x=any amino acid (SEQ ID NO: 7). In some embodiments, the stagger element may include a chemical moiety, such as glycerol, a non-nucleic acid linking moiety, a chemical modification, a modified nucleic acid, or any combination thereof.

In some embodiments, a circular polyribonucleotide includes at least one stagger element adjacent to an expression sequence. In some embodiments, the circular polyribonucleotide includes a stagger element adjacent to each expression sequence. In some embodiments, the stagger element is present on one or both sides of each expression sequence, leading to separation of the expression products, (e.g., immunogen(s) and/or adjuvant(s)). In some embodiments, the stagger element is a portion of the one or more expression sequences. In some embodiments, the circular polyribonucleotide includes one or more expression sequences (e.g., immunogen(s) and/or adjuvant(s)), and each of the one or more expression sequences is separated from a succeeding expression sequence (e.g., immunogen(s) and/or adjuvant(s) by a stagger element on the circular polyribonucleotide. In some embodiments, the stagger element prevents generation of a single polypeptide (a) from two rounds of translation of a single expression sequence or (b) from one or more rounds of translation of two or more expression sequences. In some embodiments, the stagger element is a sequence separate from the one or more expression sequences. In some embodiments, the stagger element includes a portion of an expression sequence of the one or more expression sequences.

Examples of stagger elements are described in paragraphs [0172]-[0175] of International Patent Publication No. WO2019/118919, which is hereby incorporated by reference in its entirety.

In some embodiments, the plurality of immunogens and/or adjuvants encoded by a circular ribonucleotide may be separated by an IRES between each immunogen (e.g., each immunogen is operably linked to a separate IRES). For example, a circular polyribonucleotide may include a first IRES operable linked to a first expression sequence and a second IRES operably linked to a second expression sequence. The IRES may be the same IRES between all immunogens. The IRES may be different between different immunogens.

In some embodiments, the plurality of immunogens and/or adjuvants may be separated by a 2A self-cleaving peptide. For example, a circular polyribonucleotide may encode an IRES operably linked to an open reading frame encoding a first immunogen, a 2A, and a second immunogen.

In some embodiments, the plurality of immunogens and/or adjuvants may be separated by a protease cleavage site (e.g., a furin cleavage site). For example, a circular polyribonucleotide may encode an IRES operably linked to an open reading frame encoding a first immunogen, a protease cleavage site (e.g., a furin cleavage site), and a second immunogen.

In some embodiments, the plurality of immunogens and/or adjuvants may be separated by a 2A self-cleaving peptide and a protease cleavage site (e.g., a furin cleavage site). For example, a circular polyribonucleotide may encode an IRES operably linked to an open reading frame encoding a first immunogen, a 2A, a protease cleavage site (e.g., a furin cleavage site), and a second immunogen. A circular polyribonucleotide may also encode an IRES operably linked to an open reading frame encoding a first immunogen, a protease cleavage site (e.g., a furin cleavage site), a 2A, and a second immunogen. A tandem 2A and furin cleavage site may be referred to as a furin-2A (which includes furin-2A or 2A-furin, arranged in either orientation).

Furthermore, the plurality of immunogens and/or adjuvants encoded by the circular ribonucleotide may be separated by both IRES and 2A sequences. For example, an IRES may be between one immunogen and/or adjuvant and a second immunogen and/or adjuvant while a 2A peptide may be between the second immunogen and/or adjuvant and the third immunogen and/or adjuvant. The selection of a particular IRES or 2A self-cleaving peptide may be used to control the expression level of immunogen and/or adjuvant under control of the IRES or 2A sequence. For example, depending on the IRES and or 2A peptide selected, expression on the polypeptide may be higher or lower.

To avoid production of a continuous expression product, (e.g., immunogen and/or adjuvant) while maintaining rolling circle translation, a stagger element may be included to induce ribosomal pausing during translation. In some embodiments, the stagger element is at 3′ end of at least one of the one or more expression sequences. The stagger element can be configured to stall a ribosome during rolling circle translation of the circular polyribonucleotide. The stagger element may include, but is not limited to a 2A-like, or CHYSEL (SEQ ID NO: 8) (cis-acting hydrolase element) sequence. In some embodiments, the stagger element encodes a sequence with a C-terminal consensus sequence that is X₁X₂X₃EX₅NPGP, where X₁ is absent or G or H, X₂ is absent or D or G, X₃ is D or V or I or S or M, and X₅ is any amino acid (SEQ ID NO: 9). In some embodiments, this sequence includes a non-conserved sequence of amino-acids with a strong alpha-helical propensity followed by the consensus sequence—D(V/I)ExNPGP (SEQ ID NO: 7), where x=any amino acid. Some non-limiting examples of stagger elements includes GDVESNPGP (SEQ ID NO: 10), GDIEENPGP (SEQ ID NO: 11), VEPNPGP (SEQ ID NO: 12), IETNPGP (SEQ ID NO: 13), GDIESNPGP (SEQ ID NO: 14), GDVELNPGP (SEQ ID NO: 15), GDIETNPGP (SEQ ID NO: 16), GDVENPGP (SEQ ID NO: 17), GDVEENPGP (SEQ ID NO: 18), GDVEQNPGP (SEQ ID NO: 19), IESNPGP (SEQ ID NO: 20), GDIELNPGP (SEQ ID NO: 21), HDIETNPGP (SEQ ID NO: 22), HDVETNPGP (SEQ ID NO: 23), HDVEMNPGP (SEQ ID NO: 24), GDMESNPGP (SEQ ID NO: 25), GDVETNPGP (SEQ ID NO: 26), GDIEQNPGP (SEQ ID NO: 27), and DSEFNPGP (SEQ ID NO: 28).

In some embodiments, a stagger element described herein cleaves an expression product, such as between G and P of the consensus sequence described herein. As one non-limiting example, the circular polyribonucleotide includes at least one stagger element to cleave the expression product. In some embodiments, the circular polyribonucleotide includes a stagger element adjacent to at least one expression sequence. In some embodiments, the circular polyribonucleotide includes a stagger element after each expression sequence. In some embodiments, the circular polyribonucleotide includes a stagger element is present on one or both sides of each expression sequence, leading to translation of individual peptide(s) and or polypeptide(s) from each expression sequence.

In some embodiments, a stagger element includes one or more modified nucleotides or unnatural nucleotides that induce ribosomal pausing during translation. Unnatural nucleotides may include peptide nucleic acid (PNA), Morpholino and locked nucleic acid (LNA), as well as glycol nucleic acid (GNA) and threose nucleic acid (TNA). Examples such as these are distinguished from naturally occurring DNA or RNA by changes to the backbone of the molecule. Exemplary modifications can include any modification to the sugar, the nucleobase, the internucleoside linkage (e.g., to a linking phosphate/to a phosphodiester linkage/to the phosphodiester backbone), and any combination thereof that can induce ribosomal pausing during translation. Some of the exemplary modifications provided herein are described elsewhere herein.

In some embodiments, a stagger element is present in a circular polyribonucleotide in other forms. For example, in some exemplary circular polyribonucleotides, a stagger element includes a termination element of a first expression sequence in the circular polyribonucleotide, and a nucleotide spacer sequence that separates the termination element from a first translation initiation sequence of an expression succeeding the first expression sequence. In some examples, the first stagger element of the first expression sequence is upstream of (5′ to) a first translation initiation sequence of the expression succeeding the first expression sequence in the circular polyribonucleotide. In some cases, the first expression sequence and the expression sequence succeeding the first expression sequence are two separate expression sequences in the circular polyribonucleotide. The distance between the first stagger element and the first translation initiation sequence can enable continuous translation of the first expression sequence and its succeeding expression sequence. In some embodiments, the first stagger element includes a termination element and separates an expression product of the first expression sequence from an expression product of its succeeding expression sequences, thereby creating discrete expression products. In some cases, the circular polyribonucleotide including the first stagger element upstream of the first translation initiation sequence of the succeeding sequence in the circular polyribonucleotide is continuously translated, while a corresponding circular polyribonucleotide including a stagger element of a second expression sequence that is upstream of a second translation initiation sequence of an expression sequence succeeding the second expression sequence is not continuously translated. In some cases, there is only one expression sequence in the circular polyribonucleotide, and the first expression sequence and its succeeding expression sequence are the same expression sequence. In some exemplary circular polyribonucleotides, a stagger element includes a first termination element of a first expression sequence in the circular polyribonucleotide, and a nucleotide spacer sequence that separates the termination element from a downstream translation initiation sequence. In some such examples, the first stagger element is upstream of (5′ to) a first translation initiation sequence of the first expression sequence in the circular polyribonucleotide. In some cases, the distance between the first stagger element and the first translation initiation sequence enables continuous translation of the first expression sequence and any succeeding expression sequences. In some embodiments, the first stagger element separates one round expression product of the first expression sequence from the next round expression product of the first expression sequences, thereby creating discrete expression products. In some cases, the circular polyribonucleotide including the first stagger element upstream of the first translation initiation sequence of the first expression sequence in the circular polyribonucleotide is continuously translated, while a corresponding circular polyribonucleotide including a stagger element upstream of a second translation initiation sequence of a second expression sequence in the corresponding circular polyribonucleotide is not continuously translated. In some cases, the distance between the second stagger element and the second translation initiation sequence is at least 2×, 3×, 4×, 5×, 6×, 7×, 8×, 9×, or 10× greater in the corresponding circular polyribonucleotide than a distance between the first stagger element and the first translation initiation in the circular polyribonucleotide. In some cases, the distance between the first stagger element and the first translation initiation is at least 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, 55 nt, 60 nt, 65 nt, 70 nt, 75 nt, or greater. In some embodiments, the distance between the second stagger element and the second translation initiation is at least 2 nt, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, 55 nt, 60 nt, 65 nt, 70 nt, 75 nt, or greater than the distance between the first stagger element and the first translation initiation. In some embodiments, the circular polyribonucleotide includes more than one expression sequence.

In some embodiments, a circular polyribonucleotide includes at least one cleavage sequence. In some embodiments, the cleavage sequence is adjacent to an expression sequence. In some embodiments, the cleavage sequence is between two expression sequences. In some embodiments, cleavage sequence is included in an expression sequence. In some embodiments, the circular polyribonucleotide includes between 2 and 10 cleavage sequences. In some embodiments, the circular polyribonucleotide includes between 2 and 5 cleavage sequences. In some embodiments, the multiple cleavage sequences are between multiple expression sequences; for example, a circular polyribonucleotide may include three expression sequences two cleavage sequences such that there is a cleavage sequence in between each expression sequence. In some embodiments, the circular polyribonucleotide includes a cleavage sequence, such as in an immolating circRNA or cleavable circRNA or self-cleaving circRNA. In some embodiments, the circular polyribonucleotide includes two or more cleavage sequences, leading to separation of the circular polyribonucleotide into multiple products (e.g., miRNAs, linear RNAs, smaller circular polyribonucleotide, etc.).

In some embodiments, a cleavage sequence includes a ribozyme RNA sequence. A ribozyme (from ribonucleic acid enzyme, also called RNA enzyme or catalytic RNA) is an RNA molecule that catalyzes a chemical reaction. Many natural ribozymes catalyze either the hydrolysis of one of their own phosphodiester bonds, or the hydrolysis of bonds in other RNA, but they have also been found to catalyze the aminotransferase activity of the ribosome. Catalytic RNA can be “evolved” by in vitro methods. Similar to riboswitch activity discussed above, ribozymes and their reaction products can regulate gene expression. In some embodiments, a catalytic RNA or ribozyme can be placed within a larger non-coding RNA such that the ribozyme is present at many copies within the cell for the purposes of chemical transformation of a molecule from a bulk volume. In some embodiments, aptamers and ribozymes can both be encoded in the same non-coding RNA.

In some embodiments, the cleavage sequence encodes a cleavable polypeptide linker. For example, a polyribonucleotide may encode two or more immunogens (e.g., where the two or more immunogens are encoded by a single open-reading frame (ORF)). For example, two or more immunogens may be encoded by a single open-reading frame, the expression of which is controlled by an IRES. In some embodiments, the ORF further encodes a polypeptide linker, e.g., such that the expression product of the ORF encodes two or more immunogens each separated by a sequence encoding a polypeptide linker (e.g., a linker of 5-200, 5 to 100, 5 to 50, 5 to 20, 50 to 100, or 50 to 200 amino acids). The polypeptide linker may include a cleavage site, for example, a cleavage site recognized and cleaved by a protease (e.g., an endogenous protease in a subject following administration of the polyribonucleotide to that subject). In such embodiments, a single expression product including the amino acid sequence of two or more immunogens is cleaved upon expression, such that the two or more immunogens are separated following expression. Exemplary protease cleavage sites are known to those of skill in the art, for example, amino acid sequences that act as protease cleavage sites recognized by a metalloproteinase (e.g., a matrix metalloproteinase (MMP), such as any one or more of MMPs 1-28), a disintegrin and metalloproteinase (ADAM, such as any one or more of ADAMs 2, 7-12, 15, 17-23, 28-30 and 33), a serine protease (e.g., furin), urokinase-type plasminogen activator, matriptase, a cysteine protease, an aspartic protease, or a cathepsin protease. In some embodiments, the protease is MMP9 or MMP2. In some embodiments, the protease is matriptase.

In some embodiments, a circular polyribonucleotide described herein is an immolating circular polyribonucleotide, a cleavable circular polyribonucleotide, or a self-cleaving circular polyribonucleotide. A circular polyribonucleotide can deliver cellular components including, for example, RNA, lncRNA, lincRNA, miRNA, tRNA, rRNA, snoRNA, ncRNA, siRNA, or shRNA. In some embodiments, a circular polyribonucleotide includes miRNA separated by (i) self-cleavable elements; (ii) cleavage recruitment sites; (iii) degradable linkers; (iv) chemical linkers; and/or (v) spacer sequences. In some embodiments, circRNA includes siRNA separated by (i) self-cleavable elements; (ii) cleavage recruitment sites (e.g., ADAR); (iii) degradable linkers (e.g., glycerol); (iv) chemical linkers; and/or (v) spacer sequences. Non-limiting examples of self-cleavable elements include hammerhead, splicing element, hairpin, hepatitis delta virus (HDV), Varkud Satellite (VS), and glmS ribozymes.

### Translation Initiation Sequences

In some embodiments, a circular polyribonucleotide encodes an immunogen and includes a translation initiation sequence (e.g., a start codon). In some embodiments, the translation initiation sequence includes a Kozak or Shine-Dalgarno sequence. In some embodiments, the translation initiation sequence includes a Kozak sequence. In some embodiments, the circular polyribonucleotide includes the translation initiation sequence, (e.g., Kozak sequence, adjacent to an expression sequence). In some embodiments, the translation initiation sequence is a non-coding start codon. In some embodiments, the translation initiation sequence (e.g., Kozak sequence) is present on one or both sides of each expression sequence, leading to separation of the expression products. In some embodiments, the circular polyribonucleotide includes at least one translation initiation sequence adjacent to an expression sequence. In some embodiments, the translation initiation sequence provides conformational flexibility to the circular polyribonucleotide. In some embodiments, the translation initiation sequence is within a substantially single stranded region of the circular polyribonucleotide. Further examples of translation initiation sequences are described in paragraphs [0163]-[0165] of International Patent Publication No. WO2019/118919, which is hereby incorporated by reference in its entirety.

The circular polyribonucleotide may include more than 1 start codon such as, but not limited to, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 50, at least 60 or more than 60 start codons. Translation may initiate on the first start codon or may initiate downstream of the first start codon.

In some embodiments, a circular polyribonucleotide may initiate at a codon which is not the first start codon (e.g., AUG). Translation of the circular polyribonucleotide may initiate at an alternative translation initiation sequence, such as those described in [0164] of International Patent Publication No. WO2019/118919A1, which is incorporated herein by reference in its entirety.

In some embodiments, translation is initiated by eukaryotic initiation factor 4A (eIF4A) treatment with Rocaglates (translation is repressed by blocking 43S scanning, leading to premature, upstream translation initiation and reduced protein expression from transcripts bearing the RocA-eIF4A target sequence, see for example, www.nature.com/articles/nature17978).

### Untranslated Regions

In some embodiments, a circular polyribonucleotide includes untranslated regions (UTRs). UTRs of a genomic region including a gene may be transcribed but not translated. In some embodiments, a UTR may be included upstream of the translation initiation sequence of an expression sequence described herein. In some embodiments, a UTR may be included downstream of an expression sequence described herein. In some instances, one UTR for the first expression sequence is the same as or continuous with or overlapping with another UTR for a second expression sequence.

Exemplary untranslated regions are described in paragraphs [0197]-[201] of International Patent Publication No. WO2019/118919, which is hereby incorporated by reference in its entirety.

In some embodiments, a circular polyribonucleotide includes a poly-A sequence. Exemplary poly-A sequences are described in paragraphs [0202]-[0205] of International Patent Publication No. WO2019/118919, which is hereby incorporated by reference in its entirety. In some embodiments, a circular polyribonucleotide lacks a poly-A sequence.

In some embodiments, a circular polyribonucleotide includes a UTR with one or more stretches of Adenosines and Uridines embedded within. These AU rich signatures may increase turnover rates of the expression product.

Introduction, removal, or modification of UTR AU rich elements (AREs) may be useful to modulate the stability, or immunogenicity (e.g., the level of one or more markers of an immune or inflammatory response) of the circular polyribonucleotide. When engineering specific circular polyribonucleotides, one or more copies of an ARE may be introduced to the circular polyribonucleotide and the copies of an ARE may modulate translation and/or production of an expression product. Likewise, AREs may be identified and removed or engineered into the circular polyribonucleotide to modulate the intracellular stability and thus affect translation and production of the resultant protein.

It should be understood that any UTR from any gene may be incorporated into the respective flanking regions of the circular polyribonucleotide.

In some embodiments, a circular polyribonucleotide lacks a 5′-UTR and is competent for protein expression from its one or more expression sequences. In some embodiments, the circular polyribonucleotide lacks a 3′-UTR and is competent for protein expression from its one or more expression sequences. In some embodiments, the circular polyribonucleotide lacks a poly-A sequence and is competent for protein expression from its one or more expression sequences. In some embodiments, the circular polyribonucleotide lacks a termination element and is competent for protein expression from its one or more expression sequences. In some embodiments, the circular polyribonucleotide lacks an internal ribosomal entry site and is competent for protein expression from its one or more expression sequences. In some embodiments, the circular polyribonucleotide lacks a cap and is competent for protein expression from its one or more expression sequences. In some embodiments, the circular polyribonucleotide lacks a 5′-UTR, a 3′-UTR, and an IRES, and is competent for protein expression from its one or more expression sequences. In some embodiments, the circular polyribonucleotide includes one or more of the following sequences: a sequence that encodes one or more miRNAs, a sequence that encodes one or more replication proteins, a sequence that encodes an exogenous gene, a sequence that encodes a therapeutic, a regulatory element (e.g., translation modulator, e.g., translation enhancer or suppressor), a translation initiation sequence, one or more regulatory nucleic acids that targets endogenous genes (e.g., siRNA, lncRNAs, shRNA), and a sequence that encodes a therapeutic mRNA or protein.

In some embodiments, a circular polyribonucleotide lacks a 5′-UTR. In some embodiments, the circular polyribonucleotide lacks a 3′-UTR. In some embodiments, the circular polyribonucleotide lacks a poly-A sequence. In some embodiments, the circular polyribonucleotide lacks a termination element. In some embodiments, the circular polyribonucleotide lacks an internal ribosomal entry site. In some embodiments, the circular polyribonucleotide lacks degradation susceptibility by exonucleases. In some embodiments, the fact that the circular polyribonucleotide lacks degradation susceptibility can mean that the circular polyribonucleotide is not degraded by an exonuclease, or only degraded in the presence of an exonuclease to a limited extent (e.g., that is comparable to or similar to in the absence of exonuclease).

In some embodiments, the circular polyribonucleotide is not degraded by exonucleases. In some embodiments, the circular polyribonucleotide has reduced degradation when exposed to exonuclease. In some embodiments, the circular polyribonucleotide lacks binding to a cap-binding protein. In some embodiments, the circular polyribonucleotide lacks a 5′ cap.

### Termination Elements

In some embodiments, the polyribonucleotide described herein includes at least one termination element. In some embodiments, the polyribonucleotide includes a termination element operably linked to an expression sequence. In some embodiments, the polynucleotide lacks a termination element.

In some embodiments, the polyribonucleotide includes one or more expression sequences, and each expression sequence may or may not have a termination element. In some embodiments, the polyribonucleotide includes one or more expression sequences, and the expression sequences lack a termination element, such that the polyribonucleotide is continuously translated. Exclusion of a termination element may result in rolling circle translation or continuous expression of expression product.

In some embodiments, the circular polyribonucleotide includes one or more expression sequences, and each expression sequence may or may not have a termination element. In some embodiments, the circular polyribonucleotide includes one or more expression sequences, and the expression sequences lack a termination element, such that the circular polyribonucleotide is continuously translated. Exclusion of a termination element may result in rolling circle translation or continuous expression of expression product (e.g., peptides or polypeptides, due to lack of ribosome stalling or fall-off). In such an embodiment, rolling circle translation expresses a continuous expression product through each expression sequence. In some other embodiments, a termination element of an expression sequence can be part of a stagger element. In some embodiments, one or more expression sequences in the circular polyribonucleotide includes a termination element. However, rolling circle translation or expression of a succeeding (e.g., second, third, fourth, fifth, etc.) expression sequence in the circular polyribonucleotide is performed. In such instances, the expression product may fall off the ribosome when the ribosome encounters the termination element (e.g., a stop codon, and terminates translation). In some embodiments, translation is terminated while the ribosome (e.g., at least one subunit of the ribosome) remains in contact with the circular polyribonucleotide.

In some embodiments, the circular polyribonucleotide includes a termination element at the end of one or more expression sequences. In some embodiments, one or more expression sequences includes two or more termination elements in succession. In such embodiments, translation is terminated and rolling circle translation is terminated. In some embodiments, the ribosome completely disengages with the circular polyribonucleotide. In some such embodiments, production of a succeeding (e.g., second, third, fourth, fifth, etc.) expression sequence in the circular polyribonucleotide may require the ribosome to reengage with the circular polyribonucleotide prior to initiation of translation. Generally, termination elements include an in-frame nucleotide triplet that signals termination of translation (e.g., UAA, UGA, UAG). In some embodiments, one or more termination elements in the circular polyribonucleotide are frame-shifted termination elements, such as but not limited to, off-frame or −1 and +1 shifted reading frames (e.g., hidden stop) that may terminate translation. Frame-shifted termination elements include nucleotide triples, TAA, TAG, and TGA that appear in the second and third reading frames of an expression sequence. Frame-shifted termination elements may be important in preventing misreads of mRNA, which is often detrimental to the cell. In some embodiments, the termination element is a stop codon.

In some embodiments, an expression sequence includes a poly-A sequence (e.g., at the 3′ end of an expression sequence, for example 3′ to a termination element). In some embodiments, the length of a poly-A sequence is greater than 10 nucleotides in length. In one embodiment, the poly-A sequence is greater than 15 nucleotides in length (e.g., at least or greater than about 10, 15, 20, 25, 30, 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the poly-A sequence is designed according to the descriptions of the poly-A sequence in [0202]-[0204] of International Patent Publication No. WO2019/118919A1, which is incorporated herein by reference in its entirety. In some embodiments, the expression sequence lacks a poly-A sequence (e.g., at the 3′ end of an expression sequence).

In some embodiments, a circular polyribonucleotide includes a polyA, lacks a polyA, or has a modified polyA to modulate one or more characteristics of the circular polyribonucleotide. In some embodiments, the circular polyribonucleotide lacking a polyA or having modified polyA improves one or more functional characteristics (e.g., immunogenicity (e.g., the level of one or more marker of an immune or inflammatory response), half-life, and/or expression efficiency).

Further examples of termination elements are described in paragraphs [0169]-[0170] of International Patent Publication No. WO2019/118919, which is hereby incorporated by reference in its entirety.

### Spacer Sequences

In some embodiments, a circular polyribonucleotide described herein includes a spacer sequence. In some embodiments, a polyribonucleotide described herein includes one or more spacer sequences. A spacer refers to any contiguous nucleotide sequence (e.g., of one or more nucleotides) that provides distance or flexibility between two adjacent polynucleotide regions. Spacers may be present in between any of the nucleic acid elements described herein. Spacer may also be present within a nucleic acid element described herein.

The spacer may be, e.g., at least 5 (e.g., at least 10, at least 15, at least 20) ribonucleotides in length. In some embodiments, each spacer region is at least 5 (e.g., at least 10, at least 15, at least 20) ribonucleotides in length. Each spacer region may be, e.g., from 5 to 500 (e.g., 10, 20, 30, 40, 50, 60, 70, 80, 90, 100, 150, 200, 250, 300, 350, 400, 450, or 500) ribonucleotides in length. The first spacer region, the second spacer region, or the first spacer region and the second spacer region may include a polyA sequence. The first spacer region, the second spacer region, or the first spacer region and the second spacer region may include a polyA-C sequence. In some embodiments, the first spacer region, the second spacer region, or the first spacer region and the second spacer region includes a polyA-G sequence. In some embodiments, the first spacer region, the second spacer region, or the first spacer region and the second spacer region includes a polyA-T sequence. In some embodiments, the first spacer region, the second spacer region, or the first spacer region and the second spacer region includes a random sequence.

In some embodiments, the spacer sequence can be, for example, at least 10 nucleotides in length, at least 15 nucleotides in length, or at least 30 nucleotides in length. In some embodiments, the spacer sequence is at least 7, 8, 9, 10, 11, 12, 13, 14, 15, 16, 17, 18, 19, 20, 25 or 30 nucleotides in length. In some embodiments, the spacer sequence is no more than 100, 90, 80, 70, 60, 50, 45, 40, 35 or 30 nucleotides in length. In some embodiments the spacer sequence is from 20 to 50 nucleotides in length. In certain embodiments, the spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length.

The spacer sequences can be polyA sequences, polyA-C sequences, polyC sequences, or poly-U sequences.

In some embodiments, the spacer sequences can be polyA-T, polyA-C, polyA-G, or a random sequence.

Exemplary spacer sequences are described in paragraphs [0293]-[0302] of International Patent Publication No. WO2019/118919, and this publication is hereby incorporated by reference in its entirety.

### Modifications

A circular polyribonucleotide may include one or more substitutions, insertions and/or additions, deletions, and covalent modifications with respect to reference sequences, in particular, the parent polyribonucleotide, are included within the scope of this disclosure.

In some embodiments, a circular polyribonucleotide includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc.). The one or more post-transcriptional modifications can be any post-transcriptional modification, such as any of the more than one hundred different nucleoside modifications that have been identified in RNA (Rozenski, J, Crain, P, and McCloskey, J. (1999). The RNA Modification Database: 1999 update, NUCL ACIDS REs 27: 196-97). In some embodiments, the first isolated nucleic acid includes messenger RNA (mRNA). In some embodiments, the polyribonucleotide includes at least one nucleoside selected from the group such as those described in [0311] of International Patent Publication No. WO2019/118919A1, which is incorporated herein by reference in its entirety.

A circular polyribonucleotide may include any useful modification, such as to the sugar, the nucleobase, or the internucleoside linkage (e.g., to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, a circular polyribonucleotide includes at least one N(6)methyladenosine (m6A) modification to increase translation efficiency. In some embodiments, the m6A modification can reduce immunogenicity (e.g., reduce the level of one or more marker of an immune or inflammatory response) of the circular polyribonucleotide.

In some embodiments, a modification may include a chemical or cellular induced modification.

For example, some non-limiting examples of intracellular RNA modifications are described by Lewis and Pan in “RNA modifications and structures cooperate to guide RNA-protein interactions” from NAT REVIEWS MOL CELL BIOL, 2017, 18:202-10.

In some embodiments, chemical modifications to the ribonucleotides of a circular polyribonucleotide may enhance immune evasion. The circular polyribonucleotide may be synthesized and/or modified by methods well established in the art, such as those described in CURRENT PROTOCOLS IN NUCLEIC ACID CHEMISTRY, Beaucage, S. L. et al. (Eds.), John Wiley & Sons, Inc., New York, NY, USA, which is hereby incorporated herein by reference. Modifications include, for example, end modifications, e.g., 5′ end modifications (phosphorylation (mono-, di- and tri-), conjugation, inverted linkages, etc.), 3′ end modifications (conjugation, DNA nucleotides, inverted linkages, etc.), base modifications (e.g., replacement with stabilizing bases, destabilizing bases, or bases that base pair with an expanded repertoire of partners), removal of bases (abasic nucleotides), or conjugated bases. The modified ribonucleotide bases may also include 5-methylcytidine and pseudouridine. In some embodiments, base modifications may modulate expression, immune response, stability, subcellular localization, to name a few functional effects, of the circular polyribonucleotide. In some embodiments, the modification includes a bi-orthogonal nucleotide, e.g., an unnatural base. See for example, Kimoto et al, CHEM COMMUN (Camb), 2017, 53:12309, DOI: 10.1039/c7cc06661a, which is hereby incorporated by reference.

In some embodiments, sugar modifications (e.g., at the 2′ position or 4′ position) or replacement of the sugar one or more ribonucleotides of the circular polyribonucleotide may, as well as backbone modifications, include modification or replacement of the phosphodiester linkages. Specific examples of circular polyribonucleotide include, but are not limited to, circular polyribonucleotide including modified backbones or no natural internucleoside linkages such as internucleoside modifications, including modification or replacement of the phosphodiester linkages. Circular polyribonucleotides having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, the circular polyribonucleotide will include ribonucleotides with a phosphorus atom in its internucleoside backbone.

Modified circular polyribonucleotide backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the circular polyribonucleotide may be negatively or positively charged.

The modified nucleotides, which may be incorporated into the circular polyribonucleotide, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylenephosphonates).

The a-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment. Phosphorothioate linked to the circular polyribonucleotide is expected to reduce the innate immune response through weaker binding/activation of cellular innate immune molecules.

In specific embodiments, a modified nucleoside includes an alpha-thio-nucleoside (e.g., 5′-O-(1-thiophosphate)-adenosine, 5′-O-(I-thiophosphate)-cytidine (a-thio-cytidine), 5′-O-(I-thiophosphate)-guanosine, 5′-O-(I-thiophosphate)-uridine, or 5′-O-(1-thiophosphate)-pseudouridine).

Other internucleoside linkages that may be employed according to the present disclosure, including internucleoside linkages which do not contain a phosphorous atom, are described herein.

In some embodiments, a circular polyribonucleotide may include one or more cytotoxic nucleosides. For example, cytotoxic nucleosides may be incorporated into circular polyribonucleotide, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-1-(tetrahydrofuran-2-yl)pyrimidine-2,4(IH,3H)-dione), troxacitabine, tezacitabine, 2′-deoxy-2′-methylidenecytidine (DMDC), and 6-mercaptopurine. Additional examples include fludarabine phosphate, N4-behenoyl-l-beta-D-arabinofuranosylcytosine, N4-octadecyl-1-beta-D-arabinofuranosylcytosine, N4-palmitoyl-l-(2-C-cyano-2-deoxy-beta-D-arabino-pentofuranosyl) cytosine, and P-4055 (cytarabine 5′-elaidic acid ester).

A circular polyribonucleotide may or may not be uniformly modified along the entire length of the molecule. For example, one or more or all types of nucleotides (e.g., naturally occurring nucleotides, purine or pyrimidine, or any one or more or all of A, G, U, C, 1, pU) may or may not be uniformly modified in the circular polyribonucleotide, or in a given predetermined sequence region thereof. In some embodiments, the circular polyribonucleotide includes a pseudouridine. In some embodiments, the circular polyribonucleotide includes an inosine, which may aid in the immune system characterizing the circular polyribonucleotide as endogenous versus viral RNAs. The incorporation of inosine may also mediate improved RNA stability/reduced degradation. See for example, Yu, Z. et al. (2015) RNA editing by ADAR1 marks dsRNA as “self.” Cell Res. 25, 1283-1284, which is incorporated by reference in its entirety.

In some embodiments, all nucleotides in a circular polyribonucleotide (or in a given sequence region thereof) are modified. In some embodiments, the modification may include an m6A, which may augment expression; an inosine, which may attenuate an immune response; pseudouridine, which may increase RNA stability, or translational readthrough (stagger element), an m5C, which may increase stability; and a 2,2,7-trimethylguanosine, which aids subcellular translocation (e.g., nuclear localization).

Different sugar modifications, nucleotide modifications, and/or internucleoside linkages (e.g., backbone structures) may exist at various positions in a circular polyribonucleotide. One of ordinary skill in the art will appreciate that the nucleotide analogs or other modification(s) may be located at any position(s) of the circular polyribonucleotide, such that the function of the circular polyribonucleotide is not substantially decreased. A modification may also be a non-coding region modification. The circular polyribonucleotide may include from about 1% to about 100% modified nucleotides (either in relation to overall nucleotide content, or in relation to one or more types of nucleotide, i.e., any one or more of A, G, U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

### Production Methods

The disclosure provides methods for producing circular polyribonucleotides, including, e.g., recombinant technology or chemical synthesis. For example, a DNA molecule used to produce an RNA circle can include a DNA sequence of a naturally occurring nucleic acid sequence, a modified version thereof, or a DNA sequence encoding a synthetic polypeptide not normally found in nature (e.g., chimeric molecules or fusion proteins). DNA and RNA molecules can be modified using a variety of techniques including, but not limited to, classic mutagenesis techniques and recombinant techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification or mutagenesis of selected regions of a nucleic acid sequence, synthesis of oligonucleotide mixtures and ligation of mixture groups to “build” a mixture of nucleic acid molecules and combinations thereof.

The circular polyribonucleotides may be prepared according to any available technique, including, but not limited to chemical synthesis and enzymatic synthesis. In some embodiments, a linear primary construct or linear RNA may be cyclized or concatenated to create a circRNA described herein. The mechanism of cyclization or concatenation may occur through methods such as, e.g., chemical, enzymatic, splint ligation, or ribozyme-catalyzed methods. The newly formed 5′-3′ linkage may be an intramolecular linkage or an intermolecular linkage. For example, a splint ligase, such as a SplintR® ligase, can be used for splint ligation. According to this method, a single stranded polynucleotide (splint), such as a single-stranded DNA or RNA, can be designed to hybridize with both termini of a linear polyribonucleotide, so that the two termini can be juxtaposed upon hybridization with the single-stranded splint. Splint ligase can thus catalyze the ligation of the juxtaposed two termini of the linear polyribonucleotide, generating a circRNA. In some embodiments, a DNA or RNA ligase may be used in the synthesis of the circular polynucleotides. As a non-limiting example, the ligase may be a circ ligase or circular ligase.

In another example, either the 5′ or 3′ end of the linear polyribonucleotide can encode a ligase ribozyme sequence such that during in vitro transcription, the resultant linear circRNA includes an active ribozyme sequence capable of ligating the 5′ end of the linear polyribonucleotide to the 3′ end of the linear polyribonucleotide. The ligase ribozyme may be derived from the Group I Intron, Hepatitis Delta Virus, Hairpin ribozyme or may be selected by SELEX (systematic evolution of ligands by exponential enrichment).

In another example, a linear polyribonucleotide may be cyclized or concatenated by using at least one non-nucleic acid moiety. For example, the at least one non-nucleic acid moiety may react with regions or features near the 5′ terminus or near the 3′ terminus of the linear polyribonucleotide in order to cyclize or concatenate the linear polyribonucleotide. In another example, the at least one non-nucleic acid moiety may be located in or linked to or near the 5′ terminus or the 3′ terminus of the linear polyribonucleotide. The non-nucleic acid moieties may be homologous or heterologous. As a non-limiting example, the non-nucleic acid moiety may be a linkage such as a hydrophobic linkage, ionic linkage, a biodegradable linkage, or a cleavable linkage. As another non-limiting example, the non-nucleic acid moiety is a ligation moiety. As yet another non-limiting example, the non-nucleic acid moiety may be an oligonucleotide or a peptide moiety, such as an aptamer or a non-nucleic acid linker as described herein.

In another example, linear polyribonucleotides may be cyclized or concatenated by self-splicing. In some embodiments, the linear polyribonucleotides may include loop E sequence to self-ligate. In another embodiment, the linear polyribonucleotides may include a self-circularizing intron, e.g., a 5′ and 3′ slice junction, or a self-circularizing catalytic intron such as a Group I, Group II, or Group III Introns. Nonlimiting examples of group I intron self-splicing sequences may include self-splicing permuted intron-exon sequences derived from T4 bacteriophage gene td, and the intervening sequence (IVS) rRNA of Tetrahymena, cyanobacterium Anabaena pre-tRNA-Leu gene, or a Tetrahymena pre-rRNA.

In some embodiments, the polyribonucleotide may include catalytic intron fragments, such as a 3′ half of Group I catalytic intron fragment and a 5′ half of Group I catalytic intron fragment. The first and second annealing regions may be positioned within the catalytic intron fragments. Group I catalytic introns are self-splicing ribozymes that catalyze their own excision from mRNA, tRNA, and rRNA precursors via two-metal ion phorphoryl transfer mechanism. Importantly, the RNA itself self-catalyzes the intron removal without the requirement of an exogenous enzyme, such as a ligase.

In some embodiments, the 3′ half of Group I catalytic intron fragment and the 5′ half of Group I catalytic intron fragment are from a cyanobacterium Anabaena pre-tRNA-Leu gene, or a Tetrahymena pre-rRNA.

In some embodiments, the 3′ half of Group I catalytic intron fragment and the 5′ half of Group I catalytic intron fragment are from a Cyanobacterium Anabaena pre-tRNA-Leu gene, and the 3′ exon fragment includes the first annealing region and the 5′ exon fragment includes the second annealing region. The first annealing region may include, e.g., from 5 to 50, e.g., from 10 to 15 (e.g., 10, 11, 12, 13, 14, or 15) ribonucleotides and the second annealing region may include, e.g., from 5 to 50, e.g., from 10 to 15 (e.g., 10, 11, 12, 13, 14, or 15) ribonucleotides.

In some embodiments, the 3′ half of Group I catalytic intron fragment and the 5′ half of Group I catalytic intron fragment are from a Tetrahymena pre-rRNA, and the 3′ half of Group I catalytic intron fragment includes the first annealing region and the 5′ exon fragment includes the second annealing region. In some embodiments, the 3′ exon includes the first annealing region and the 5′ half of Group I catalytic intron fragment includes the second annealing region. The first annealing region may include, e.g., from 6 to 50, e.g., from 10 to 16 (e.g., 10, 11, 12, 13, 14, 15, or 16) ribonucleotides, and the second annealing region may include, e.g., from 6 to 50, e.g., from 10 to 16 (e.g., 10, 11, 12, 13, 14, 15, or 16) ribonucleotides.

In some embodiments, the 3′ half of Group I catalytic intron fragment and the 5′ half of Group I catalytic intron fragment are from a cyanobacterium Anabaena pre-tRNA-Leu gene, a Tetrahymena pre-rRNA, or a T4 phage td gene.

In some embodiments, the 3′ half of Group I catalytic intron fragment and the 5′ Group I catalytic intron fragment are from a T4 phage td gene. The 3′ exon fragment may include the first annealing region and the 5′ half of Group I catalytic intron fragment may include the second annealing region. The first annealing region may include, e.g., from 2 to 16, e.g., 10 to 16 (e.g., 2, 3, 4, 5, 6, 7, 8, 9, 10, 11, 12, 13, 14, 15, or 16) ribonucleotides, and the second annealing region may include, e.g., from 2 to 16, e.g., 10 to 16 (e.g., 2, 3, 4, 5, 6, 7, 8, 9, 10, 11, 12, 13, 14, 15, or 16) ribonucleotides.

In some embodiments, the 3′ half of Group I catalytic intron fragment is the 5′ terminus of the linear polynucleotide.

In some embodiments, the 5′ half of Group I catalytic intron fragment is the 3′ terminus of the linear polyribonucleotide.

In some embodiments, the 3′ half of Group I catalytic intron fragment has at least 80% (e.g., at least 85%, 90%, 95%, 97%, 99%, or 100%) sequence identity to the sequence of 5′-AACAACAGATAACTTACAGCTAGTCGGAAGGTGCAGAGACTCGACGGGAGCTACCCTAACGTCAAG ACGAGGGTAAAGAGAGAGTCCAATTCTCAAAGCCAATAGGCAGTAGCGAAAGCTGCGGGAGAATG-3′ (SEQ ID NO: 97).

In some embodiments, the 5′ half of Group I catalytic intron fragment has at least 80% (e.g., at least 85%, 90%, 95%, 97%, 99%, or 100%) sequence identity to the sequence of 5′-AAATAATTGAGCCTTAGAGAAGAAATTCTTTAAGTGGATGCTCTCAAACTCAGGGAAACCTAAATCTA GCTATAGACAAGGCAATCCTGAGCCAAGCCGAAGTAGTAATTAGTAAGTT-3′ (SEQ ID NO: 98).

In some embodiments, the 3′ half of Group I catalytic intron fragment has the sequence of SEQ ID NO: 97 and the 5′ half of Group I catalytic intron fragment has the sequence of SEQ ID NO: 98. In some embodiments, the 3′ half of Group I catalytic intron fragment has at least 80% (e.g., at least 85%, 90%, 95%, 97%, 99%, or 100%) sequence identity to the sequence of 5′-CTTCTGTTGATATGGATGCAGTTCACAGACTAAATGTCGGTCGGGGAAGATGTATTCTTCTCATAAGA TATAGTCGGACCTCTCCTTAATGGGAGCTAGCGGATGAAGTGATGCAACACTGGAGCCGCTGGGAA CTAATTTGTATGCGAAAGTATATTGATTAGTTTTGGAGTACTCG-3′ (SEQ ID NO: 99).

In some embodiments, the 5′ half of Group I catalytic intron fragment has at least 80% (e.g., at least 85%, 90%, 95%, 97%, 99%, or 100%) sequence identity to the sequence of 5′-AAATAGCAATATTTACCTTTGGAGGGAAAAGTTATCAGGCATGCACCTGGTAGCTAGTCTTTAAACCA ATAGATTGCATCGGTTTAAAAGGCAAGACCGTCAAATTGCGGGAAAGGGGTCAACAGCCGTTCAGTA CCAAGTCTCAGGGGAAACTTTGAGATGGCCTTGCAAAGGGTATGGTAATAAGCTGACGGACATGGT CCTAACCACGCAGCCAAGTCCTAAGTCAACAGAT-3′ (SEQ ID NO: 100).

In some embodiments, the 3′ half of Group I catalytic intron fragment has the sequence of SEQ ID NO: 99 and the 5′ half of Group I catalytic intron fragment has the sequence of SEQ ID NO: 100.

In some embodiments, the 3′ half of Group I catalytic intron fragment has at least 80% (e.g., at least 85%, 90%, 95%, 97%, 99%, or 100%) sequence identity to the sequence of 5′-GGTTCTACATAAATGCCTAACGACTATCCCTTTGGGGAGTAGGGTCAAGTGACTCGAAACGATAGAC AACTTGCTTTAACAAGTTGGAGATATAGTCTGCTCTGCATGGTGACATGCAGCTGGATATAATTCCGG GGTAAGATTAACGACCTTATCTGAACATAATG-3′ (SEQ ID NO: 101).

In some embodiments, the 5′ half of Group I catalytic intron fragment has at least 80% (e.g., at least 85%, 90%, 95%, 97%, 99%, or 100%) sequence identity to the sequence of 5′-TAATTGAGGCCTGAGTATAAGGTGACTTATACTTGTAATCTATCTAAACGGGGAACCTCTCTAGTAGA CAATCCCGTGCTAAATTGTAGGACT-3′ (SEQ ID NO: 102).

In some embodiments, the 3′ half of Group I catalytic intron fragment has the sequence of SEQ ID NO: 101 and the 5′ half of Group I catalytic intron fragment has the sequence of SEQ ID NO: 102.

In some embodiments, the 3′ half of Group I catalytic intron fragment has at least 80% (e.g., at least 85%, 90%, 95%, 97%, 99%, or 100%) sequence identity to the sequence of 5′-TAAACAACTAACAGCTTTAGAAGGTGCAGAGACTAGACGGGAGCTACCCTAACGGATTCAGCCGAG GGTAAAGGGATAGTCCAATTCTCAACATCGCGATTGTTGATGGCAGCGAAAGTTGCAGAGAGAATGA AAATCCGCTGACTGTAAAGGTCGTGAGGGTTCGAGTCCCTCCGCCCCCA-3′ (SEQ ID NO: 103).

In some embodiments, the 5′ half of Group I catalytic intron fragment has at least 80% (e.g., at least 85%, 90%, 95%, 97%, 99%, or 100%) sequence identity to the sequence of 5′-ACGGTAGACGCAGCGGACTTAGAAAACTGGGCCTCGATCGCGAAAGGGATCGAGTGGCAGCTCTCA AACTCAGGGAAACCTAAAACTTTAAACATTMAAGTCATGGCAATCCTGAGCCAAGCTAAAGC-3′ (SEQ ID NO: 104).

In some embodiments, the 3′ half of Group I catalytic intron fragment has the sequence of SEQ ID NO: 103 and the 5′ half of Group I catalytic intron fragment has the sequence of SEQ ID NO: 104.

In some embodiments, the 3′ half of Group I catalytic intron fragment has at least 80% (e.g., at least 85%, 90%, 95%, 97%, 99%, or 100%) sequence identity to the sequence of 5′-TTAAACTCAAAATTTAAAATCCCAAATTCAAAATTCCGGGAAGGTGCAGAGACTCGACGGGAGCTAC CCTAACGTAAAGCCGAGGGTAAAGGGAGAGTCCAATTCTCAAAGCCTGAAGTTGCTGAAGCAACAA GGCAGTAGTGAAAGCTGCGAGAGAATGAAAATCCGTTGACTGTAAAAAGTCGTGGGGGTTCAAGTC CCCCCACCCCC-3′ (SEQ ID NO: 105).

In some embodiments, the 5′ half of Group I catalytic intron fragment has at least 80% (e.g., at least 85%, 90%, 95%, 97%, 99%, or 100%) sequence identity to the sequence of 5′-ATGGTAGACGCTACGGACTTAGAAAACTGAGCCTTGATAGAGAAATCTTTTAAGTGGAAGCTCTCAAA TTCAGGGAAACCTAAATCTGAATACAGATATGGCAATCCTGAGCCAAGCCCAGAAAATTTAGACTTGA GATTTGATTTTGGAG-3′ (SEQ ID NO: 106).

In some embodiments, the 3′ half of Group I catalytic intron fragment has the sequence of SEQ ID NO: 105 and the 5′ half of Group I catalytic intron fragment has the sequence of SEQ ID NO: 106.

In some embodiments, the 3′ half of Group I catalytic intron fragment has at least 80% (e.g., at least 85%, 90%, 95%, 97%, 99%, or 100%) sequence identity to the sequence of 5′-GGCTTTCAATTTGAAATCAGAAATTCAAAATTCAGGGAAGGTGCAGAGACTCGACGGGAGCTACCCT AACGTAAAGGCGAGGGTAAAGGGAGAGTCCAATTCTTAAAGCCTGAAGTTGTGCAAGCAACAAGGC AACAGTGAAAGCTGTGGAAGAATGAAAATCCGTTGACCTTAAACGGTCGTGGGGGTTCAAGTCCCCC CACCCCC-3′ (SEQ ID NO: 107).

In some embodiments, the 5′ half of Group I catalytic intron fragment has at least 80% (e.g., at least 85%, 90%, 95%, 97%, 99%, or 100%) sequence identity to the sequence of 5′-ATGGTAGACGCTACGGACTTAGAAAACTGAGCCTTGATAGAGAAATCTTTCAAGTGGAAGCTCTCAA ATTCAGGGAAACCTAAATCTGAATACAGATATGGCAATCCTGAGCCAAGCCCGGAAATTTTAGAATCA AGATTTTATTTT-3′ (SEQ ID NO: 108).

In some embodiments, the 3′ half of Group I catalytic intron fragment has the sequence of SEQ ID NO: 107 and the 5′ half of Group I catalytic intron fragment has the sequence of SEQ ID NO: 108.

In some embodiments, the 3′ half of Group I catalytic intron fragment has at least 80% (e.g., at least 85%, 90%, 95%, 97%, 99%, or 100%) sequence identity to the sequence of 5′-AGAAATGGAGAAGGTGTAGAGACTGGAAGGCAGGCACCCTAACGTTAAAGGCGAGGGTGAAGGGA CAGTCCAGACCACAAACCAGTAAATCTGGGCAGCGAAAGCTGTAGATGGTAAGCATAACCCGAAGG TCAGTGGTTCAAATCCACTTCCCGCCACCAAATTAAAAAAACAATAA-3′ (SEQ ID NO: 109).

In some embodiments, the 5′ half of Group I catalytic intron fragment has at least 80% (e.g., at least 85%, 90%, 95%, 97%, 99%, or 100%) sequence identity to the sequence of 5′-AGAAATGGAGAAGGTGTAGAGACTGGAAGGCAGGCACCCTAACGTTAAAGGCGAGGGTGAAGGGA CAGTCCAGACCACAAACCAGTAAATCTGGGCAGCGAAAGCTGTAGATGGTAAGCATAACCCGAAGG TCAGTGGTTCAAATCCACTTCCCGCCACCAAATTAAAAAAACAATAA-3′ (SEQ ID NO: 110).

In some embodiments, the 3′ half of Group I catalytic intron fragment has the sequence of SEQ ID NO: 109 and the 5′ half of Group I catalytic intron fragment has the sequence of SEQ ID NO: 110.

In some embodiments, the 3′ half of Group I catalytic intron fragment has at least 80% (e.g., at least 85%, 90%, 95%, 97%, 99%, or 100%) sequence identity to the sequence of 5′-ACAACAGATAACTTACTAACTTACAGCTAGTCGGAAGGTGCAGAGACTCGACGGGAGCTACCCTAAC GTCAAGACGAGGGTAAAGAGAGAGTCCAATTCTCAAAGCCAATAGGCAGTAGCGAAAGCTGCGGGA GAATGAAAATCCGTAGCGTCTAAACGGTCGTGTGGGTTCAAGTCCCTCCACCCCCA-3′ (SEQ ID NO: 111).

In some embodiments, the 5′ half of Group I catalytic intron fragment has at least 80% (e.g., at least 85%, 90%, 95%, 97%, 99%, or 100%) sequence identity to the sequence of 5′-AGACGCTACGGACTTAAATAATTGAGCCTTAGAGAAGAAATTCTTTAAGTGGATGCTCTCAAACTCAG GGAAACCTAAATCTAGCTATAGACAAGGCAATCCTGAGCCAAGCCGAAGTAGTAATTAGTAAGTTAG TAAGTT-3′ (SEQ ID NO: 112).

In some embodiments, the 3′ half of Group I catalytic intron fragment has the sequence of SEQ ID NO: 111 and the 5′ half of Group I catalytic intron fragment has the sequence of SEQ ID NO: 112.

In some embodiments, the 3′ half of Group I catalytic intron fragment has at least 80% (e.g., at least 85%, 90%, 95%, 97%, 99%, or 100%) sequence identity to the sequence of 5′-AACAACAGATAACTTACTAGTTACTAGTCGGAAGGTGCAGAGACTCGACGGGAGCTACCCTAACGTC AAGACGAGGGTAAAGAGAGAGTCCAATTCTCAAAGCCAATAGGCAGTAGCGAAAGCTGCGGGAGAA TGAAAATCCGTAGCGTCTAAACGGTCGTGTGGGTTCAAGTCCCTCCACCCCCA-3′ (SEQ ID NO: 113).

In some embodiments, the 5′ half of Group I catalytic intron fragment has at least 80% (e.g., at least 85%, 90%, 95%, 97%, 99%, or 100%) sequence identity to the sequence of 5′-AGACGCTACGGACTTAAATAATTGAGCCTTAGAGAAGAAATTCTTTAAGTGGATGCTCTCAAACTCAG GGAAACCTAAATCTAGCTATAGACAAGGCAATCCTGAGCCAAGCCGAAGTAGTAATTAGTAAGTT-3′ (SEQ ID NO: 114).

In some embodiments, the 3′ half of Group I catalytic intron fragment has the sequence of SEQ ID NO: 113 and the 5′ half of Group I catalytic intron fragment has the sequence of SEQ ID NO: 114.

In another example, a linear polyribonucleotide may be cyclized or concatenated by a non-nucleic acid moiety that causes an attraction between atoms, molecular surfaces at, near, or linked to the 5′ and 3′ ends of the linear polyribonucleotide. The one or more linear polyribonucleotides may be cyclized or concatenated by intermolecular forces or intramolecular forces. Non-limiting examples of intermolecular forces include dipole-dipole forces, dipole-induced dipole forces, induced dipole-induced dipole forces, Van der Waals forces, and London dispersion forces. Non-limiting examples of intramolecular forces include covalent bonds, metallic bonds, ionic bonds, resonant bonds, agnostic bonds, dipolar bonds, conjugation, hyperconjugation and antibonding.

In another example, the linear polyribonucleotide may comprise a ribozyme RNA sequence near the 5′ terminus and near the 3′ terminus. The ribozyme RNA sequence may covalently link to a peptide when the sequence is exposed to the remainder of the ribozyme. The peptides covalently linked to the ribozyme RNA sequence near the 5′ terminus and the 3′terminus may associate with each other, thereby causing a linear polyribonucleotide to cyclize or concatenate. In another example, the peptides covalently linked to the ribozyme RNA near the 5′ terminus and the 3′ terminus may cause the linear primary construct or linear mRNA to cyclize or concatenate after being subjected to ligated using various methods known in the art such as, but not limited to, protein ligation. Non-limiting examples of ribozymes for use in the linear primary constructs or linear polyribonucleotides of the present invention or a non-exhaustive listing of methods to incorporate or covalently link peptides are described in US patent application No. US20030082768, the contents of which is here in incorporated by reference in its entirety.

In yet another example, chemical methods of circularization may be used to generate the circular polyribonucleotide. Such methods may include but are not limited to click chemistry (e.g., alkyne and azide-based methods, or clickable bases), olefin metathesis, phosphoramidate ligation, hemiaminal-imine crosslinking, base modification, and any combination thereof.

In another example, the circular polyribonucleotide may be produced using a deoxyribonucleotide template transcribed in a cell-free system (e.g., by in vitro transcription) to a produce a linear RNA. The linear polyribonucleotide produces a splicing-compatible polyribonucleotide, which may be self-spliced to produce a circular polyribonucleotide.

In some embodiments, the disclosure provides a method of producing a circular polyribonucleotide (e.g., in a cell-free system) by providing a linear polyribonucleotide; and self-splicing linear polyribonucleotide under conditions suitable for splicing of the 3′ and 5′ splice sites of the linear polyribonucleotide; thereby producing a circular polyribonucleotide.

In some embodiments, the disclosure provides a method of producing a circular polyribonucleotide by providing a deoxyribonucleotide encoding the linear polyribonucleotide; transcribing the deoxyribonucleotide in a cell-free system to produce the linear polyribonucleotide; optionally purifying the splicing-compatible linear polyribonucleotide; and self-splicing the linear polyribonucleotide under conditions suitable for splicing of the 3′ and 5′ splice sites of the linear polyribonucleotide, thereby producing a circular polyribonucleotide.

In some embodiments, the disclosure provides a method of producing a circular polyribonucleotide by providing a deoxyribonucleotide encoding a linear polyribonucleotide; transcribing the deoxyribonucleotide in a cell-free system to produce the linear polyribonucleotide, wherein the transcribing occurs in a solution under conditions suitable for splicing of the 3′ and 5′ splice sites of the linear polyribonucleotide, thereby producing a circular polyribonucleotide. In some embodiments, the linear polyribonucleotide comprises a 5′ split-intron and a 3′ split-intron (e.g., a self-splicing construct for producing a circular polyribonucleotide). In some embodiments, the linear polyribonucleotide comprises a 5′ annealing region and a 3′ annealing region.

Suitable conditions for in vitro transcriptions and or self-splicing may include any conditions (e.g., a solution or a buffer, such as an aqueous buffer or solution) that mimic physiological conditions in one or more respects. In some embodiments, suitable conditions include between 0.1-100 mM Mg2+ ions or a salt thereof (e.g., 1-100 mM, 1-50 mM, 1-20 mM, 5-50 mM, 5-20 mM, or 5-15 mM). In some embodiments, suitable conditions include between 1-1000 mM K+ ions or a salt thereof such as KCl (e.g., 1-1000 mM, 1-500 mM, 1-200 mM, 50-500 mM, 100-500 mM, or 100-300 mM). In some embodiments, suitable conditions include between 1-1000 mM Cl− ions or a salt thereof such as KCl (e.g., 1-1000 mM, 1-500 mM, 1-200 mM, 50-500 mM, 100-500 mM, or 100-300 mM). In some embodiments, suitable conditions include between 0.1-100 mM Mn2+ ions or a salt thereof such as MnCl2 (e.g., 0.1-100 mM, 0.1-50 mM, 0.1-20 mM, 0.1-10 mM, 0.1-5 mM, 0.1-2 mM, 0.5-50 mM, 0.5-20 mM, 0.5-15 mM, 0.5-5 mM, 0.5-2 mM, or 0.1-10 mM). In some embodiments, suitable conditions include dithiothreitol (DTT) (e.g., 1-1000 μM, 1-500 μM, 1-200 μM, 50-500 μM, 100-500 μM, 100-300 μM, 0.1-100 mM, 0.1-50 mM, 0.1-20 mM, 0.1-10 mM, 0.1-5 mM, 0.1-2 mM, 0.5-50 mM, 0.5-20 mM, 0.5-15 mM, 0.5-5 mM, 0.5-2 mM, or 0.1-10 mM). In some embodiments, suitable conditions include between 0.1 mM and 100 mM ribonucleoside triphosphate (NTP) (e.g., 0.1-100 mM, 0.1-50 mM, 0.1-10 mM, 1-100 mM, 1-50 mM, or 1-10 mM). In some embodiments, suitable conditions include a pH of 4 to 10 (e.g., pH of 5 to 9, pH of 6 to 9, or pH of 6.5 to 8.5). In some embodiments, suitable conditions include a temperature of 4° C. to 50° C. (e.g., 10° C. to 40° C., 15° C. to 40° C., 20° C. to 40° C., or 30° C. to 40° C.),

In some embodiments the linear polyribonucleotide is produced from a deoxyribonucleic acid, e.g., a deoxyribonucleic acid described herein, such as a DNA vector, a linearized DNA vector, or a cDNA. In some embodiments, the linear polyribonucleotide is transcribed from the deoxyribonucleic acid by transcription in a cell-free system (e.g., in vitro transcription).

In another example, the circular polyribonucleotide may be produced in a cell, e.g., a prokaryotic cell or a eukaryotic cell. In some embodiments, an exogenous polyribonucleotide is provided to a cell (e.g., a linear polyribonucleotide described herein or a DNA molecule encoding for the transcription of a linear polyribonucleotide described here). The linear polyribonucleotides may be transcribed in the cell from an exogenous DNA molecule provided to the cell. The linear polyribonucleotide may be transcribed in the cell from an exogenous recombinant DNA molecule transiently provided to the cell. In some embodiments, the exogenous DNA molecule does not integrate into the cell's genome. In some embodiments, the linear polyribonucleotide is transcribed in the cell from a recombinant DNA molecule that is incorporated into the cell's genome.

In some embodiments, the cell is a prokaryotic cell. In some embodiments, the prokaryotic cell including the polyribonucleotides described herein may be a bacterial cell or an archaeal cell. For example, the prokaryotic cell including the polyribonucleotides described herein may be E coli, halophilic archaea (e.g., Haloferax volcaniii), Sphingomonas, cyanobacteria (e.g., Synechococcus elongatus, Spirulina (Arthrospira) spp., and Synechocystis spp.), Streptomyces, actinomycetes (e.g., Nonomuraea, Kitasatospora, or Thermobifida), Bacillus spp. (e.g., Bacillus subtilis, Bacillus anthracis, Bacillus cereus), betaproteobacteria (e.g., Burkholderia), alphaproteobacterial (e.g., Agrobacterium), Pseudomonas (e.g., Pseudomonas putida), and enterobacteria. The prokaryotic cells may be grown in a culture medium. The prokaryotic cells may be contained in a bioreactor.

The cell may be a eukaryotic cell. In some embodiments, the eukaryotic cell is a unicellular eukaryotic cell. In some embodiments, the unicellular eukaryotic is a unicellular fungal cell such as a yeast cell (e.g., Saccharomyces cerevisiae and other Saccharomyces spp., Brettanomyces spp., Schizosaccharomyces spp., Torulaspora spp, and Pichia spp.). In some embodiments, the unicellular eukaryotic cell is a unicellular animal cell. A unicellular animal cell may be a cell isolated from a multicellular animal and grown in culture, or the daughter cells thereof. In some embodiments, the unicellular animal cell may be dedifferentiated. In some embodiments, the unicellular eukaryotic cell is a unicellular plant cell. A unicellular plant cell may be a cell isolated from a multicellular plant and grown in culture, or the daughter cells thereof. In some embodiments, the unicellular plant cell may be dedifferentiated. In some embodiments, the unicellular plant cell is from a plant callus. In embodiments, the unicellular cell is a plant cell protoplast. In some embodiments, the unicellular eukaryotic cell is a unicellular eukaryotic algal cell, such as a unicellular green alga, a diatom, a euglenid, or a dinoflagellate.

Non-limiting examples of unicellular eukaryotic algae of interest include Dunaliella salina, Chlorella vulgaris, Chlorella zofingiensis, Haematococcus pluvialis, Neochloris oleoabundans and other Neochloris spp., Protosiphon botryoides, Botryococcus braunii, Cryptococcus spp., Chlamydomonas reinhardtii and other Chlamydomonas spp. In some embodiments, the unicellular eukaryotic cell is a protist cell. In some embodiments, the unicellular eukaryotic cell is a protozoan cell.

In some embodiments, the eukaryotic cell is a cell of a multicellular eukaryote. For example, the multicellular eukaryote may be selected from the group consisting of a vertebrate animal, an invertebrate animal, a multicellular fungus, a multicellular alga, and a multicellular plant. In some embodiments, the eukaryotic organism is a human. In some embodiments, the eukaryotic organism is a non-human vertebrate animal. In some embodiments, the eukaryotic organism is an invertebrate animal. In some embodiments, the eukaryotic organism is a multicellular fungus. In some embodiments, the eukaryotic organism is a multicellular plant. In embodiments, the eukaryotic cell is a cell of a human or a cell of a non-human mammal such as a non-human primate (e.g., monkeys, apes), ungulate (e.g., bovids including cattle, buffalo, bison, sheep, goat, and musk ox; pig; camelids including camel, llama, and alpaca; deer, antelope; and equids including horse and donkey), carnivore (e.g., dog, cat), rodent (e.g., rat, mouse, guinea pig, hamster, squirrel), or lagomorph (e.g., rabbit, hare). In embodiments, the eukaryotic cell is a cell of a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots). In embodiments, the eukaryotic cell is a cell of an arthropod (e.g., insects, arachnids, crustaceans), a nematode, an annelid, a helminth, or a mollusc. In embodiments, the eukaryotic cell is a cell of a multicellular plant, such as an angiosperm plant (which can be a dicot or a monocot) or a gymnosperm plant (e.g., a conifer, a cycad, a gnetophyte, a Ginkgo), a fern, horsetail, clubmoss, or a bryophyte. In embodiments, the eukaryotic cell is a cell of a eukaryotic multicellular alga.

The eukaryotic cells may be grown in a culture medium. The eukaryotic cells may be contained in a bioreactor.

Examples of bioreactors include, without limitation, stirred tank (e.g., well mixed) bioreactors and tubular (e.g., plug flow) bioreactors, airlift bioreactors, membrane stirred tanks, spin filter stirred tanks, vibromixers, fluidized bed reactors, and membrane bioreactors. The mode of operating the bioreactor may be a batch or continuous processes. A bioreactor is continuous when the reagent and product streams are continuously being fed and withdrawn from the system. A batch bioreactor may have a continuous recirculating flow, but no continuous feeding of reagents or product harvest. Some methods of the present disclosure are directed to large-scale production of circular polyribonucleotides. For large-scale production methods, the method may be performed in a volume of 1 liter (L) to 50 L, or more (e.g., 5 L, 10 L, 15 L, 20 L, 25 L, 30 L, 35 L, 40 L, 45 L, 50 L, or more). In some embodiments, the method may be performed in a volume of 5 L to 10 L, 5 L to 15 L, 5 L to 20 L, 5 L to 25 L, 5 L to 30 L, 5 L to 35 L, 5 L to 40 L, 5 L to 45 L, 10 L to 15 L, 10 L to 20 L, 10 L to 25 L, 20 L to 30 L, 10 L to 35 L, 10 L to 40 L, 10 L to 45 L, 10 L to 50 L, 15 L to 20 L, 15 L to 25 L, 15 L to 30 L, 15 L to 35 L, 15 L to 40 L, 15 L to 45 L, or 15 to 50 L. In some embodiments, a bioreactor may produce at least 1 g of circular RNA. In some embodiments, a bioreactor may produce 1-200 g of circular RNA (e.g., 1-10 g, 1-20 g, 1-50 g, 10-50 g, 10-100 g, 50-100 g, of 50-200 g of circular RNA). In some embodiments, the amount produced is measured per liter (e.g., 1-200 g per liter), per batch or reaction (e.g., 1-200 g per batch or reaction), or per unit time (e.g., 1-200 g per hour or per day). In some embodiments, more than one bioreactor may be utilized in series to increase the production capacity (e.g., one, two, three, four, five, six, seven, eight, or nine bioreactors may be used in series).

Methods of making the circular polyribonucleotides described herein are described in, for example, Khudyakov & Fields, Artificial DNA: Methods and Applications, CRC Press (2002); in Zhao, SYNTHETIC BIOLOGY: TOOLS AND APPLICATIONS, (First Edition), Academic Press (2013); and Egli & Herdewijn, CHEMISTRY AND BIOLOGY OF ARTIFICIAL NUCLEIC ACIDS, (First Edition), Wiley-VCH (2012).

Various methods of synthesizing circular polyribonucleotides are also described elsewhere (see, e.g., U.S. Pat. Nos. 6,210,931, 5,773,244, 5,766,903, 5,712,128, 5,426,180, US Publication No. US20100137407, International Publication No. WO1992001813, International Publication No. WO2010084371, and Petkovic et al., Nucleic Acids Res. 43:2454-65 (2015); the contents of each of which are herein incorporated by reference in their entirety).

In some embodiments, the circular polyribonucleotide is purified, e.g., free ribonucleic acids, linear or nicked RNA, DNA, proteins, etc. are removed. In some embodiments, the circular polyribonucleotides may be purified by any known method commonly used in the art. Examples of nonlimiting purification methods include, column chromatography, gel excision, size exclusion, etc.

### Immunization

In some embodiments, methods of the disclosure include immunizing a subject with an immunogenic composition including a circular polyribonucleotide as disclosed herein. In some embodiments, an immunogen is expressed from the circular polyribonucleotide. In some embodiments, immunization induces an immune response in a subject against the immunogen expressed from the circular polyribonucleotide. In some embodiments, immunization induces an immune response in a subject (e.g., induces the production of antibodies that bind to the immunogen expressed from the circular polyribonucleotide). In some embodiments, immunization is for the purpose of treating or preventing a disease, disorder, or condition in the subject (e.g., a human subject). In some embodiments, immunization is for the purpose of producing antibodies in the subject (e.g., producing antibodies for purification, such as in a non-human mammal). In some embodiments, an immunogenic composition includes the circular polyribonucleotide and a diluent, carrier, first adjuvant or a combination thereof in a single composition. In some embodiments, the subject is further immunized with a second adjuvant. In some embodiments, the subject is further immunized with a second immunogenic composition.

The subject is immunized with one or more immunogenic composition(s) including any number of circular polyribonucleotides. The subject is immunized with, for example, one or more immunogenic composition(s) including at least 1 circular polyribonucleotide. A subject is immunized with, for example, one or more immunogenic composition(s) including at least 2, at least 3, at least 4, at least 5, at least 6, at least 7, at least 8, at least 9, at least 10, at least 11, at least 12, at least 13, at least 14, at least 15, at least 20 different circular polyribonucleotides, or more different circular polyribonucleotides. In some embodiments, a subject is immunized with one or more immunogenic composition(s) including at most 1 circular polyribonucleotide. In some embodiments, a subject is immunized with one or more immunogenic composition(s) including about 1 circular polyribonucleotide. In some embodiments, a subject is immunized with one or more immunogenic composition(s) including about 1-20, 1-15, 1-10, 1-9, 1-8, 1-7, 1-6, 1-5, 1-4, 1-3, 1-2, 2-20, 2-15, 2-10, 2-9, 2-8, 2-7, 2-6, 2-5, 2-4, 2-3, 3-20, 3-15, 3-10, 3-9, 3-8, 3-7, 3-6, 3-5, 3-4, 4-20, 4-15, 4-10, 4-9, 4-8, 4-7, 4-6, 4-5, 4-4, 4-3, 5-20, 5-15, 5-10, 5-9, 5-8, 5-7, 5-6, 5-10, 10-15, or 15-20 different circular polyribonucleotides. Different circular polyribonucleotides have different sequences from each other. For example, they can include or encode different immunogens, overlapping immunogens, similar immunogens, or the same immunogens (for example, with the same or different regulatory elements, initiation sequences, promoters, termination elements, or other elements of the disclosure). In cases where a subject is immunized with one or more immunogenic composition(s) including two or more different circular polyribonucleotides, the two or more different circular polyribonucleotides can be in the same or different immunogenic compositions and immunized at the same time or at different times. The immunogenic compositions including two or more different circular polyribonucleotides can be administered to the same anatomical location or different anatomical locations.

In some embodiments, an immunogenic composition includes a circular polyribonucleotide and a diluent, a carrier, a first adjuvant, or a combination thereof. In a particular embodiment, an immunogenic composition includes a circular polyribonucleotide described herein and a carrier or a diluent free of any carrier. In some embodiments, an immunogenic composition including a circular polyribonucleotide with a diluent free of any carrier is used for naked delivery of the circular polyribonucleotide to a subject. In another particular embodiment, an immunogenic composition includes a circular polyribonucleotide described herein and a first adjuvant.

In certain embodiments, a subject is further administered a second adjuvant. An adjuvant enhances the innate immune response, which in turn, enhances the adaptive immune response in a subject. An adjuvant can be any adjuvant as discussed below. In certain embodiments, an adjuvant is formulated with the circular polyribonucleotide as a part of an immunogenic composition. In certain embodiments, an adjuvant is not part of an immunogenic composition including the circular polyribonucleotide. In certain embodiments, an adjuvant is administered separately from an immunogenic composition including the circular polyribonucleotide. In this aspect, the adjuvant is co-administered (e.g., administered simultaneously) or administered at a different time than an immunogenic composition including the circular polyribonucleotide to the subject. For example, the adjuvant is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, after an immunogenic composition including the circular polyribonucleotide. In some embodiments, the adjuvant is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, before an immunogenic composition including the circular polyribonucleotide. For example, the adjuvant is administered 1, 2, 3, 4, 5, 6, 7, 14, 21, 28, 35, 42, 49, 56, 63, 70, 77, or 84 days, or any day therebetween, after an immunogenic composition including the circular polyribonucleotide. In some embodiments, the adjuvant is administered 1, 2, 3, 4, 5, 6, 7, 14, 21, 28, 35, 42, 49, 56, 63, 70, 77, or 84 days, or any day therebetween, before an immunogenic composition including the circular polyribonucleotide. The adjuvant is administered to the same anatomical location or different anatomical location as the immunogenic composition including the circular polyribonucleotide.

In some embodiments, a subject is further immunized with a second agent, e.g., a vaccine (as described below) that is not a circular polyribonucleotide. The vaccine is co-administered (e.g., administered simultaneously) or administered at a different time than an immunogenic composition including the circular polyribonucleotide to the subject. For example, the vaccine is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, after an immunogenic composition including the circular polyribonucleotide. In some embodiments, the vaccine is administered 1 minute, 5 minutes, 10 minutes, 15 minutes, 30 minutes, 45 minutes, 60 minutes, 90 minutes, 2 hours, 3 hours, 4 hours, 5 hours, 6 hours, 7 hours, 8 hours, 9 hours, 10 hours, 12 hours, 14 hours, 16 hours, 18 hours, 20 hours, 22 hours, or 24 hours, or any minute or hour therebetween, before an immunogenic composition including the circular polyribonucleotide. For example, the vaccine is administered 1, 2, 3, 4, 5, 6, 7, 14, 21, 28, 35, 42, 49, 56, 63, 70, 77, or 84 days, or any day therebetween, after an immunogenic composition including the circular polyribonucleotide. In some embodiments, the vaccine is administered 1, 2, 3, 4, 5, 6, 7, 14, 21, 28, 35, 42, 49, 56, 63, 70, 77, or 84 days, or any day therebetween, before an immunogenic composition including the circular polyribonucleotide.

A subject can be immunized with an immunogenic composition, adjuvant, vaccine (e.g., protein subunit vaccine), or a combination thereof any suitable number of times to achieve a desired response. For example, a prime-boost immunization strategy can be utilized to elicit systemic and/or mucosal immunity. A subject can be immunized with an immunogenic composition, adjuvant, vaccine (e.g., protein subunit vaccine), or a combination thereof, of the disclosure, for example, at least 1, at least 2, at least 3, at least 4, at least 5, at least 6, at least 7, at least 8, at least 9, at least 10, or at least 15 times, or more.

In some embodiments, a subject can be immunized with an immunogenic composition, adjuvant, vaccine (e.g., protein subunit vaccine), or a combination thereof, of the disclosure at most 2, at most 3, at most 4, at most 5, at most 6, at most 7, at most 8, at most 9, at most 10, at most 15, or at most 20 times, or less.

In some embodiments, a subject can be immunized with an immunogenic composition, adjuvant, vaccine (e.g., protein subunit vaccine), or a combination thereof, of the disclosure about 1, 2, 3, 4, 5, 6, 7, 8, 9, 10, 15, or 20 times.

In some embodiments, a subject can be immunized with an immunogenic composition, adjuvant, vaccine (e.g., protein subunit vaccine), or a combination thereof, of the disclosure once. In some embodiments, a subject can be immunized with an immunogenic composition, adjuvant, vaccine (e.g., protein subunit vaccine), or a combination thereof, of the disclosure twice. In some embodiments, a subject can be immunized with an immunogenic composition, adjuvant, vaccine (e.g., protein subunit vaccine), or a combination thereof, of the disclosure three times. In some embodiments, a subject can be immunized with an immunogenic composition, adjuvant, vaccine (e.g., protein subunit vaccine), or a combination thereof, of the disclosure four times. In some embodiments, a subject can be immunized with an immunogenic composition, adjuvant, vaccine (e.g., protein subunit vaccine), or a combination thereof, of the disclosure five times. In some embodiments, a subject can be immunized with an immunogenic composition, adjuvant, vaccine (e.g., protein subunit vaccine), or a combination thereof, of the disclosure seven times.

Suitable time intervals can be selected for spacing two or more immunizations. The time intervals can apply to multiple immunizations with the same immunogenic composition, adjuvant, or vaccine (e.g., protein subunit vaccine), or combination thereof, for example, the same immunogenic composition, adjuvant, or vaccine (e.g., protein subunit vaccine), or combination thereof, can be administered in the same amount or a different amount, via the same immunization route or a different immunization route. The time intervals can apply to multiple immunizations with a different immunogenic composition, adjuvant, or vaccine (e.g., protein subunit vaccine), or combination thereof, for example, a different immunogenic composition, adjuvant, or vaccine (e.g., protein subunit vaccine), or combination thereof, can be administered in the same amount or a different amount, via the same immunization route or a different immunization route. The time intervals can apply to immunizations with different agents, for example, a first immunogenic composition including a first circular polyribonucleotide and a second immunogenic composition including a second circular polyribonucleotide. The time intervals can apply to immunizations with different agents, for example, a first immunogenic composition including a first circular polyribonucleotide and a second immunogenic composition including a protein immunogen (e.g., a protein subunit). In some examples, about 1, 2, 3, 4, 5, 6, 7, 8, 9, 10, 11, 12, 14, 16, 17, 18, 20, 22, 24, 26, 28, 30, 32, 34, 36-, 40-, 48-, or 72-hours elapse between two immunizations. In some embodiments, about 1, 2, 3, 4, 5, 6, 7, 8, 9, 10, 11, 12, 14, 16, 17, 18, 20, 21-, 24-, 28-, or 30-days elapse between two immunizations. In some embodiments, about 1, 2, 3, 4, 5-, 6-, 7-, or 8-weeks elapse between two immunizations. In some embodiments, about 1, 2, 3, 4, 5-, 6-, 7-, or 8-months elapse between two immunizations.

In some embodiments, at least 1, at least 2, at least 3, at least 4, at least 5, at least 6, at least 7, at least 8, at least 9, at least 10, at least 15, at least 20, at least 24, at least 36, or at least 72 hours, or more elapse between two immunizations. In some embodiments, at most 1, at most 2, at most 3, at most 4, at most 5, at most 6, at most 7, at most 8, at most 9, at most 10, at most 15, at most 20, at most 24, at most 36, or at most 72 hours, or less elapse between two immunizations.

In some embodiments, at least 1, at least 2, at least 3, at least 4, at least 5, at least 6, at least 7, at least 8, at least 9, at least 10, at least 15, at least 20, at least 21, at least 22, at least 23, at least 24, at least 25, at least 26 at least 27, at least 28, at least 29, or at least 30 days, or more, elapse between two immunizations. In some embodiments, at most 2, at most 3, at most 4, at most 5, at most 6, at most 7, at most 8, at most 9, at most 10, at most 15, at most 20, at most 21, at most 22, at most 23, at most 24, at most 25, at most 26, at most 27, at most 28, at most 29, at most 30, at most 32, at most 34, or at most 36 days, or less elapse between two immunizations.

In some embodiments, at least 1, at least 2, at least 3, at least 4, at least 5, at least 6, at least 7, or at least 8 weeks, or more elapse between two immunizations. In some embodiments, at most 2, at most 3, at most 4, at most 5, at most 6, at most 7, at most 8 weeks, or less elapse between two immunizations.

In some embodiments, at least 1, at least 2, at least 3, at least 4, at least 5, at least 6, at least 7, or at least 8 months, or more elapse between two immunizations. In some embodiments, at most 2, at most 3, at most 4, at most 5, at most 6, at most 7, at most 8 months, at most 9 months, at most 10 months, at most 11 months, or at most 12 months or less elapse between two immunizations.

In some embodiments, the method includes pre-administering to the subject an agent to improve immunogenic responses to a circular polyribonucleotide including a sequence encoding an immunogen.

In some embodiments, the agent is the immunogen as disclosed herein (e.g., a protein immunogen). For example, the method includes administering the protein immunogen from 1 to 7 days prior to administration of the circular polyribonucleotide including the sequence encoding the protein immunogen.

In some embodiments, the protein immunogen is administered 1, 2, 3, 4, 5, 6, or 7 days prior to administration of the circular polyribonucleotide including the sequence encoding the protein immunogen.

The protein immunogen may be administered as a protein preparation, encoded in a plasmid (pDNA), presented in a virus-like particle (VLP), formulated in a lipid nanoparticle, or the like.

In some embodiments, the method includes administering to the subject an agent to improve immunogenic responses to a circular polyribonucleotide including a sequence encoding an immunogen after the subject has been administered the circular polyribonucleotide including a sequence encoding an immunogen. In some embodiments, the agent is the immunogen as disclosed herein (e.g., a protein immunogen). In some embodiments, the circular polyribonucleotide includes a sequence encoding a protein immunogen. For example, the method includes administering the protein immunogen within 1 year (e.g., within 11 months, 10 months, 9 months, 8 months, 7 months, 6 months, 5 months, 4 months, 3 months, 2 months, and 1 month) of administering the circular polyribonucleotide including a sequence encoding the immunogen to the subject. In some embodiments, the method includes administering any one of the circular polyribonucleotides described herein or any one of the immunogenic compositions described herein and a protein subunit to the subject.

In some embodiments, the protein immunogen has the same amino acid sequence as the immunogen encoded by circular polyribonucleotide. For example, the polypeptide immunogen may correspond to (e.g., shares 90%, 95%, 96%, 97%, 98%, or 100%) amino acid sequence identity with a polypeptide immunogen encoded by a sequence of the circular polyribonucleotide. In some embodiments, the protein immunogen has a different amino acid sequence from the amino acid sequence of the immunogen encoded by the circular polyribonucleotide. For example, the polypeptide immunogen may share less than 90% (e.g., 80%, 70%, 30%, 20%, or 10%) amino acid sequence identity with the polypeptide immunogen encoded by a sequence of the circular polyribonucleotide.

A subject can be immunized with an immunogenic composition, an adjuvant, or a vaccine (e.g., protein subunit vaccine), or a combination thereof, at any suitable number anatomical sites. The same immunogenic composition, an adjuvant, a vaccine (e.g., protein subunit vaccine), or a combination thereof can be administered to multiple anatomical sites, different immunogenic compositions including the same or different circular polyribonucleotides, adjuvants, vaccines (e.g., protein subunit vaccine) or a combination thereof can be administered to different anatomical sites, different immunogenic compositions including the same or different circular polyribonucleotides, adjuvants, vaccines (e.g., protein subunit vaccines) or a combination thereof can be administered to the same anatomical site, or any combination thereof. For example, an immunogenic composition including a circular polyribonucleotide can be administered in to two different anatomical sites, and/or an immunogenic composition including a circular polyribonucleotide can be administered to one anatomical site, and an adjuvant can be administered to a different anatomical site.

Immunization at any two or more anatomical routes can be via the same route of immunization (e.g., intramuscular) or by two or more routes of immunization. In some embodiments, an immunogenic composition including a circular polyribonucleotide, an adjuvant, or a vaccine (e.g., protein subunit vaccine), or a combination thereof, of the disclosure is immunized to at least 1, at least 2, at least 3, at least 4, at least 5, or at least 6 anatomical sites of a subject. In some embodiments, an immunogenic composition including a circular polyribonucleotide, an adjuvant, or a vaccine (e.g., protein subunit vaccine), or a combination thereof, of the disclosure is immunized to at most 2, at most 3, at most 4, at most 5, at most 6, at most 7, at most 8, at most 9, or at most 10 anatomical sites of the subject, or less. In some embodiments, an immunogenic composition including a circular polyribonucleotide, or an adjuvant of the disclosure is immunized to 1, 2, 3, 4, 5, 6, 7, 8, 9, 10 anatomical sites of a subject.

Immunization can be by any suitable route. Non-limiting examples of immunization routes include intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intrasternal, intracerebral, intraocular, intralesional, intracerebroventricular, intracisternal, or intraparenchymal, e.g., injection and infusion. In some cases, immunization can be via inhalation. Two or more immunizations can be done by the same route or by different routes.

Any suitable amount of a circular polyribonucleotide can be administered to a subject of the disclosure. For example, a subject can be immunized with at least about 1 ng, at least about 10 ng, at least about 100 ng, at least about 1 μg, at least about 10 μg, at least about, at least about 100 μg, at least about 1 mg, at least about 10 mg, at least about 100 mg, or at least about 1 g of a circular polyribonucleotide. In some embodiments, a subject can be immunized with at most about 1 ng, at most about 10 ng, at most about 100 ng, at most about 1 μg, at most about 10 μg, at most about, at most about 100 μg, at most about 1 mg, at most about 10 mg, at most about 100 mg, or at most about 1 g of a circular polyribonucleotide. In some embodiments, a subject can be immunized with about 1 ng, about 10 ng, about 100 ng, about 1 μg, about 10 μg, about, about 100 μg, about 1 mg, about 10 mg, about 100 mg, or about 1 g of a circular polyribonucleotide.

In some embodiments, the method further includes evaluating the subject for antibody response to the immunogen. In some embodiments, the evaluating is before and/or after administration of the circular polyribonucleotide including a sequence encoding an immunogen.

### Production and Purification of Antibodies

Immunization of a subject with a polyribonucleotide described herein (e.g., a polyribonucleotide encoding an immunogen including a multimerization domain) may induce the production of antibodies in the subject that bind to the immunogen expressed from the circular polyribonucleotide (e.g., produce antibodies). In some embodiments, immunization is for the purpose of producing antibodies in the subject (e.g., a human or a non-human animal) which are quantified or purified from the subject (e.g., for diagnostic or therapeutic use). Thereby, circular polyribonucleotides of the present invention may be used in methods of producing polyclonal or monoclonal antibodies (e.g., polyclonal or monoclonal antibodies).

For example, the disclosure provides administering a circular polyribonucleotide described herein (e.g., encoding an immunogen including a multimerization domain) to a non-human animal (e.g., a non-human mammal, such as a goat, pig, rabbit, rat, mouse, llama, camel, horse, donkey, or bovine (cow)). The circular polyribonucleotide may be administered according to any composition, formulation, route or administration, amount, or dosing regimen described herein (e.g., optionally with an adjuvant, administered in the same composition or as part of a dosing regimen). In some embodiments, the non-human animal has a humanized immune system (e.g., a bovine having a humanized immune system).

Plasma including polyclonal antibodies produced from immunogenic compositions including circular polyribonucleotides as disclosed herein can be collected from a subject that was immunized with the circular polyribonucleotide. These polyclonal antibodies can be quantified (e.g., for diagnostic purposes in a human subject) or purified (e.g., for use in a method of treatment or for the development of monoclonal antibodies). Plasma can be collected by methods known to those of skill in the art, e.g., via plasmapheresis. Plasma can be collected from the same subject once or multiple times, for example, multiple times each a given period of time after an immunization, multiple times after an immunization, multiple times in between immunizations, or any combination thereof.

Antibodies, or fragments thereof, (e.g., polyclonal antibodies, such as human or humanized polyclonal antibodies) that bind specifically to an immunogen including a multimerization domain may be produced by the methods described herein. Antibodies, or fragments thereof, may be purified from blood (e.g., from blood plasma or blood serum) by methods known to those of skill in the art.

Polyclonal antibodies may be purified from plasma using techniques well known to those of skill in the art. For example, plasma is pH-adjusted to 4.8 (e.g., with dropwise addition of 20% acetic acid), fractionated by caprylic acid at a caprylic acid/total protein ratio of 1.0, and then clarified by centrifugation (e.g., at 10,000 g for 20 min at room temperature). The supernatant containing polyclonal antibodies (e.g., IgG polyclonal antibodies) is neutralized to pH 7.5 with 1 M tris, 0.22 μM filtered, and affinity-purified with an anti-human immunoglobulin-specific column (e.g., anti-human IgG light chain-specific column).

The polyclonal antibodies are further purified by passage over an affinity column that specifically binds impurities, for example, non-human antibodies from the non-human animal. The polyclonal antibodies are stored in a suitable buffer, for example, a sterile-filtered buffer consisting of 10 mM glutamic acid monosodium salt, 262 mM D-sorbitol, and Tween (0.05 mg/ml) (pH 5.5). The quantity and concentration of the purified polyclonal antibodies are determined. HPLC size exclusion chromatography is conducted to determine whether aggregates or multimers are present. In some embodiments, the human polyclonal antibodies are purified from a non-human animal having a humanized immune system according to Beigel, J H et al., LANCET INFECT. DIS., 18:410-18 (2018), including Supplementary appendix), which is herein incorporated by reference in its entirety.

The disclosure also provides methods of producing antibodies in a human subject, e.g., for therapeutic treatment and/or diagnosis. For example, the disclosure provides a method of quantifying a level of anti-immunogen antibodies in a subject following administration of a circular polyribonucleotide or immunogenic composition described herein. Quantification may be performed by methods known in the art (e.g., performing an antibody titer), for example by obtaining a blood sample from the subject and quantifying the anti-immunogen antibody level using standard techniques, such as an enzyme-linked immunoassay (ELISA). Antibodies may also be purified by methods known to those of skill in the art.

### Adjuvants

An adjuvant enhances the immune responses (humoral and/or cellular) elicited in a subject who receives the adjuvant and/or an immunogenic composition including the adjuvant. In some embodiments, an adjuvant is administered to a subject as disclosed herein. In some embodiments, an adjuvant is used in the methods described herein to produce an immune response as described herein. In a particular embodiment, an adjuvant is used to promote an immune response in a subject against an immunogen expressed from a circular polyribonucleotide. In some embodiments, an adjuvant and polyribonucleotide are co-administered in separate compositions. In some embodiments, an adjuvant is mixed or formulated with a polyribonucleotide in a single composition and administered to a subject. In some embodiments, an adjuvant and circular polyribonucleotide are co-administered in separate compositions. In some embodiments, an adjuvant is mixed or formulated with a circular polyribonucleotide in a single composition to obtain an immunogenic composition that is administered to a subject.

An adjuvant may be a component of a circular polyribonucleotide (e.g., a polyribonucleotide sequence), may be polypeptide adjuvant encoded by an expression sequence of a polyribonucleotide, may be a molecule (e.g., a small molecule, polypeptide, or nucleic acid molecule) that is not encoded by the polyribonucleotide. An adjuvant may be formulated with a polyribonucleotide in the same pharmaceutical composition. An adjuvant may be administered separately (e.g., as a separate pharmaceutical composition) in combination with a polyribonucleotide.

In some embodiments, the adjuvant is encoded by the circular polyribonucleotide. In some embodiments, the circular polyribonucleotide encodes more than one adjuvant. For example, the circular polyribonucleotide encodes between 2 and 100 adjuvants. In some embodiments, the circular polyribonucleotide encodes between 2 and 10 adjuvants. In some embodiments, the circular polyribonucleotide encodes 2 adjuvants. One or more of the adjuvants encoded by a circular polyribonucleotide may include an N-terminal signal sequence, e.g., that directs the expressed polypeptide adjuvant to the secretory pathway. In some embodiments, the polyribonucleotide encodes 3 adjuvants. In some embodiments, the polyribonucleotide encodes 4 adjuvants. In some embodiments, the polyribonucleotide encodes 5 adjuvants. In some embodiments, the adjuvant is encoded by the same polyribonucleotide that encodes one or more immunogens. The adjuvant(s) and immunogen(s) may be co-delivered on the same polyribonucleotide. In some embodiments, the adjuvant encoded by the polyribonucleotide is a sequence (e.g., a polyribonucleotide sequence) that is an innate immune system stimulator. The innate immune system stimulator sequence may include at least 5, at least 10, at least 20, at least 50, at least 100, or at least 500 ribonucleotides. The innate immune system stimulator sequence may include between 5 and 1000, between 10 and 500, between 20 and 500, between 10 and 100, between 20 and 100, between 20 and 50, between 100 and 500, between 500 and 1000, or between 10 and 1000 ribonucleotides. For example, a sequence that is an innate immune system stimulator may be selected from a GU-rich motif, an AU-rich motif, a structured region including dsRNA, or an aptamer.

Adjuvants may be a TH1 adjuvant and/or a TH2 adjuvant. Further adjuvants contemplated by this disclosure include, but are not limited to, one or more of the following:

Mineral-containing compositions. Mineral-containing compositions suitable for use as adjuvants in the disclosure include mineral salts, such as aluminum salts, and calcium salts. The disclosure includes mineral salts such as hydroxides (e.g., oxyhydroxides), phosphates (e.g., hydroxyphosphates, orthophosphates), sulphates, etc., or mixtures of different mineral compounds, with the compounds taking any suitable form (e.g., gel, crystalline, amorphous, etc.). Calcium salts include calcium phosphate (e.g., the “CAP”). Aluminum salts include hydroxides, phosphates, sulfates, and the like.

Oil emulsion compositions. Oil-emulsion compositions suitable for use as adjuvants in the disclosure include squalene-water emulsions, such as MF59 (5% Squalene, 0.5% Tween 80 and 0.5% Span, formulated into submicron particles using a microfluidizer), AS03 (α-tocopherol, squalene and polysorbate 80 in an oil-in-water emulsion), Montanide formulations (e.g., Montanide ISA 51, Montanide ISA 720), incomplete Freunds adjuvant (IFA), complete Freund's adjuvant (CFA), and incomplete Freund's adjuvant (IFA).

Small molecules. Small molecules suitable for use as adjuvants in the disclosure include imiquimod or 847, resiquimod or R848, and gardiquimod.

Polymeric nanoparticles. Polymeric nanoparticles suitable for use as an adjuvant in the disclosure include poly(a-hydroxy acids), polyhydroxy butyric acids, polylactones (including polycaprolactones), polydioxanones, polyvalerolactone, polyorthoesters, polyanhydrides, polycyanoacrylates, tyrosine-derived polycarbonates, polyvinyl-pyrrolidinones or polyester-amides, and combinations thereof.

Saponin (i.e., a glycoside, polycyclic aglycones attached to one or more sugar side chains). Saponin formulations suitable for use as an adjuvant in the disclosure include purified formulations, such as QS21, as well as lipid formulations, such as ISCOMs and ISCOMs matrix. QS21 is marketed as STIMULON™. Saponin formulations may also include a sterol, such as cholesterol. Combinations of saponins and cholesterols can be used to form unique particles called immune-stimulating complexes (ISCOMs). ISCOMs typically also include a phospholipid such as phosphatidylethanolamine or phosphatidylcholine. Any known saponin can be used in ISCOMs. Preferably, the ISCOM includes one or more of QuilA, QHA & QHC. Optionally, the ISCOMS may be devoid of additional detergent.

Lipopolysaccharides. Adjuvants suitable for use in the disclosure include non-toxic derivatives of enterobacterial lipopolysaccharide (LPS). Such derivatives include monophosphoryl lipid A (MPLA), glucopyranosyl lipid A (GLA) and 3-O-deacylated MPL (3dMPL). 3dMPL is a mixture of 3 De-O-acylated monophosphoryl lipid A with 4, 5 or 6 acylated chains. Other non-toxic LPS derivatives include monophosphoryl lipid A mimics, such as aminoalkyl glucosaminide phosphate derivatives e.g., RC-529.

Liposomes. Liposomes suitable for use as an adjuvant in the disclosure include virosomes and CAF01.

Lipid nanoparticles. Adjuvants suitable for use in the disclosure include lipid nanoparticles (LNPs) and their components.

Lipopeptides (i.e., compounds including one or more fatty acid residues and two or more amino acid residues). Lipopeptide suitable for use as an adjuvant in the disclosure include Pam2 (Pam2CSK4) and Pam3 (Pam3CSK4).

Glycolipids. Glycolipids suitable for use as an adjuvant in the disclosure include cord factor (trehalose dimycolate).

Peptides and peptidoglycans derived from (synthetic or purified) gram-negative or gram-positive bacteria, such as MDP (N-acetyl-muramyl-L-alanyl-D-isoglutamine) are suitable for use as an adjuvant in the disclosure

Carbohydrates (carbohydrate containing) or polysaccharides suitable for use as an adjuvant include dextran (e.g., branched microbial polysaccharide), dextran-sulfate, lentinan, zymosan, beta-glucan, deltin, mannan, and chitin.

RNA based adjuvants. RNA based adjuvants suitable for use in the disclosure are poly IC, poly IC:LC, hairpin RNAs with or without a 5′triphosphate, viral sequences, polyU containing sequence, dsRNA natural or synthetic RNA sequences (e.g., poly I:C), and nucleic acid analogs (e.g., cyclic GMP-AMP or other cyclic dinucleotides e.g., cyclic di-GMP, immunostimulatory base analogs e.g., C8-substituted and N7,C8-disubstituted guanine ribonucleotides). In some embodiments, the adjuvant is the linear polyribonucleotide counterpart of the circular polyribonucleotide described herein.

DNA based adjuvants. DNA based adjuvants suitable for use in the disclosure include CpGs (e.g., CpG1018), dsDNA, and natural or synthetic immunostimulatory DNA sequences.

Proteins or peptides. Proteins and peptides suitable for use as an adjuvant in the disclosure include flagellin-fusion proteins, MBL (mannose-binding lectin), cytokines, and chemokines.

Viral particles. Viral particles suitable for use as an adjuvant include virosomes (phospholipid cell membrane bilayer).

An adjuvant for use in the disclosure may be bacterial derived, such as a flagellin, LPS, or a bacterial toxin (e.g., enterotoxins (protein), e.g., heat-labile toxin or cholera toxin). An adjuvant for use in the disclosure may be a hybrid molecule such as CpG conjugated to imiquimod. An adjuvant for use in the disclosure may be a fungal or oomycete microbe-associated molecular patterns (MAMPs), such as chitin or beta-glucan. In some embodiments, an adjuvant is an inorganic nanoparticle, such as gold nanorods or silica-based nanoparticles (e.g., mesoporous silica nanoparticles (MSN)). In some embodiments, an adjuvant is a multi-component adjuvant or adjuvant system, such as AS01 (AS01B), AS03, AS04 (MLP5+ alum), alum (mixture of aluminum hydroxide and magnesium hydroxide), aluminum hydroxide, magnesium hydroxide, CFA (complete Freund's adjuvant: IFA+peptiglycan+trehalose dimycolate), CAF01 (two component system of cationic liposome vehicle (dimethyl dioctadecyl-ammonium (DDA)) stabilized with a glycolipid immunomodulator (trehalose 6,6-dibehenate (TDB), which can be a synthetic variant of cord factor located in the mycobacterial cell wall).

Cytokines. An adjuvant may be a partial or full-length DNA encoding a cytokine such as, a pro-inflammatory cytokine (e.g., GM-CSF, IL-1 alpha, IL-1 beta, TGF-beta, TNF-alpha, TNF-beta), Th-1 inducing cytokines (e.g., IFN-gamma, IL-2, IL-12, IL-15, IL-18), or Th-2 inducing cytokines (e.g., IL-4, IL-5, IL-6, IL-10, IL-13).

Chemokines. An adjuvant may be a partial or full-length DNA or RNA (e.g., circular polyribonucleotide or mRNA) encoding a chemokine such as, MCP-1, MIP-1 alpha, MIP-1 beta, Rantes, or TCA-3.

An adjuvant may be a partial or full-length DNA encoding a costimulatory molecule, such as CD80, CD86, CD40-L, CD70, or CD27.

An adjuvant may be a partial or full length DNA or RNA (e.g., circular polyribonucleotide or mRNA) encoding for an innate immune system stimulator (partial, full-length, or mutated) such as TLR4, TLR3, TLR3, TLR9, TLR7, TLR8, TLR7, RIG-I/DDX58, or MDA-5/IFIH1; or a constitutively active (ca) innate immune stimulator, such as caTLR4, caTLR3, caTLR3, caTLR9, caTLR7, caTLR8, caTLR7, caRIG-I/DDX58, or caMDA-5/IFIH1.

An adjuvant may be a partial or full-length DNA or RNA (e.g., circular polyribonucleotide or mRNA) encoding for an adaptor or signaling molecule, such as STING (e.g., caSTING), TRIF, TRAM, MyD88, IPS1, ASC, MAVS, MAPKs, IKK-alpha, IKK complex, TBK1, beta-catenin, and caspase 1.

An adjuvant may be a partial or full-length DNA or RNA (e.g., circular polyribonucleotide or mRNA) encoding for a transcriptional activator, such as a transcription activator that can upregulate an immune response (e.g., AP1, NF-kappa B, IRF3, IRF7, IRF1, or IRFS). An adjuvant may be a partial or full-length DNA encoding for a cytokine receptor, such as IL-2beta, IFN-gamma, or IL-6.

An adjuvant may be a partial or full-length DNA or RNA (e.g., circular polyribonucleotide or mRNA) encoding for a bacterial component, such as flagellin or MBL.

An adjuvant may be a partial or full-length DNA or RNA (e.g., circular polyribonucleotide or mRNA) encoding for any component of the innate immune system.

In some embodiments, a subject is administered a circular polyribonucleotide encoding one or more immunogens in combination with an adjuvant (e.g., an adjuvant that is a separate molecular entity from the circular polyribonucleotide or an adjuvant that is encoded on a separate polyribonucleotide). The term “in combination with” as used throughout the description includes any two compositions administered as part of a therapeutic regimen. This may include, for example, a polyribonucleotide and an adjuvant formulated as a single pharmaceutical composition. This also includes, for example, a polyribonucleotide and an adjuvant administered to a subject as separate compositions according to a defined therapeutic or dosing regimen. An adjuvant may be administered to a subject before, at substantially the same time, or after the administration of a polyribonucleotide. An adjuvant may be administered within 1 day, 2 days, 5 days, 10 days, 20 days, 1 month, 2 months, 3 months, 4 months, 5 months, or 6 months before or after administration of a polyribonucleotide. An adjuvant may be administered by the same route of administration (e.g., intradermally, intramuscularly, subcutaneously, intravenously, intraperitoneally, topically, or orally) or a different route than a polyribonucleotide.

### Delivery

A circular polyribonucleotide described herein may be included in pharmaceutical compositions with a carrier or without a carrier.

Pharmaceutical compositions described herein may be formulated for example including a carrier, such as a pharmaceutical carrier and/or a polymeric carrier, e.g., a liposome, and delivered by known methods to a subject in need thereof (e.g., a human or non-human agricultural or domestic animal, e.g., cattle, dog, cat, horse, poultry). Such methods include, but not limited to, transfection (e.g., lipid-mediated, cationic polymers, calcium phosphate, dendrimers); electroporation or other methods of membrane disruption (e.g., nucleofection), viral delivery (e.g., lentivirus, retrovirus, adenovirus, AAV), microinjection, microprojectile bombardment (“gene gun”), fugene, direct sonic loading, cell squeezing, optical transfection, protoplast fusion, impalefection, magnetofection, exosome-mediated transfer, lipid nanoparticle-mediated transfer, and any combination thereof. Methods of delivery are also described, e.g., in Gori et al., Delivery and Specificity of CRISPR/Cas9 Genome Editing Technologies for Human Gene Therapy. Human Gene Therapy. July 2015, 26(7): 443-451. doi:10.1089/hum.2015.074; and Zuris et al. Cationic lipid-mediated delivery of proteins enables efficient protein-based genome editing in vitro and in vivo. NAT BIOTECHNOL. 2014 Oct. 30; 33(1):73-80.

In some embodiments, circular polyribonucleotides may be delivered in a “naked” delivery formulation. A naked delivery formulation delivers a circular polyribonucleotide to a cell without the aid of a carrier and without covalent modification of the circular polyribonucleotide or partial or complete encapsulation of the circular polyribonucleotide.

A naked delivery formulation is a formulation that is free from a carrier and wherein the circular polyribonucleotide is without a covalent modification that binds a moiety that aids in delivery to a cell and the circular polyribonucleotide is not partially or completely encapsulated. In some embodiments, the circular polyribonucleotide is not covalently bound to a moiety, such as a protein, small molecule, a particle, a polymer, or a biopolymer that aids in delivery to a cell. In some embodiments, circular polyribonucleotides may be delivered in a delivery formulation with protamine or a protamine salt (e.g., protamine sulfate).

A polyribonucleotide without covalent modification that binds to a moiety that aids in delivery to a cell may not contain a modified phosphate group. For example, a polyribonucleotide without covalent modification that binds to a moiety that aids in delivery to a cell may not contain phosphorothioate, phosphoroselenates, boranophosphates, boranophosphate esters, hydrogen phosphonates, phosphoramidates, phosphorodiamidates, alkyl or aryl phosphonates, or phosphotriesters.

In some embodiments, a naked delivery formulation may be free of any or all of: transfection reagents, cationic carriers, carbohydrate carriers, nanoparticle carriers, or protein carriers. For example, a naked delivery formulation may be free from phytoglycogen octenyl succinate, phytoglycogen beta-dextrin, anhydride-modified phytoglycogen beta-dextrin, lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane (DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-l-propanaminium trifluoroacetate (DOSPA), 3B-[N-(N\N′-Dimethylaminoethane)-carbamoyl]Cholesterol Hydrochloride (DC-Cholesterol HCl), diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride (DODAC), human serum albumin (HSA), low-density lipoprotein (LDL), high-density lipoprotein (HDL), or globulin.

A naked delivery formulation may include a non-carrier excipient. In some embodiments, a non-carrier excipient may include an inactive ingredient that does not exhibit an active cell-penetrating effect. In some embodiments, a non-carrier excipient may include a buffer, for example PBS. In some embodiments, a non-carrier excipient may be a solvent, a non-aqueous solvent, a diluent, a suspension aid, a surface-active agent, an isotonic agent, a thickening agent, an emulsifying agent, a preservative, a polymer, a peptide, a protein, a cell, a hyaluronidase, a dispersing agent, a granulating agent, a disintegrating agent, a binding agent, a buffering agent, a lubricating agent, or an oil.

In some embodiments, a naked delivery formulation may include a diluent, such as a parenterally acceptable diluent. A diluent (e.g., a parenterally acceptable diluent) may be a liquid diluent or a solid diluent. In some embodiments, a diluent (e.g., a parenterally acceptable diluent) may be an RNA solubilizing agent, a buffer, or an isotonic agent. Examples of an RNA solubilizing agent include water, ethanol, methanol, acetone, formamide, and 2-propanol. Examples of a buffer include 2-(N-morpholino)ethanesulfonic acid (MES), Bis-Tris, 2-[(2-amino-2-oxoethyl)-(carboxymethyl)amino]acetic acid (ADA), N-(2-Acetamido)-2-aminoethanesulfonic acid (ACES), piperazine-N,N′-bis(2-ethanesulfonic acid) (PIPES), 2-[[1,3-dihydroxy-2-(hydroxymethyl)propan-2-yl]amino]ethanesulfonic acid (TES), 3-(N-morpholino)propanesulfonic acid (MOPS), 4-(2-hydroxyethyl)-1-piperazineethanesulfonic acid (HEPES), Tris, Tricine, Gly-Gly, Bicine, or phosphate. Examples of an isotonic agent include glycerin, mannitol, polyethylene glycol, propylene glycol, trehalose, or sucrose.

In some embodiments, the formulation includes a cell-penetrating agent. In some embodiments, the formulation is a topical formulation and includes a cell-penetrating agent. The cell-penetrating agent can include organic compounds such as alcohols having one or more hydroxyl function groups. In some cases, the cell-penetrating agent includes an alcohol such as, but not limited to, monohydric alcohols, polyhydric alcohols, unsaturated aliphatic alcohols, and alicyclic alcohols. The cell-penetrating agent can include one or more of methanol, ethanol, isopropanol, phenoxyethanol, triethanolamine, phenethyl alcohol, butanol, pentanol, cetyl alcohol, ethylene glycol, propylene glycol, denatured alcohol, benzyl alcohol, specially denatured alcohol, glycol, stearyl alcohol, cetearyl alcohol, menthol, polyethylene glycols (PEG)-400, ethoxylated fatty acids, or hydroxyethylcellulose. In certain embodiments, the cell-penetrating agent includes ethanol. The cell-penetrating agents can include any cell-penetrating agent in any amount or in any formulation as described in WO 2020/180751 or WO 2020/180752, which are hereby incorporated by reference in their entirety.

In some embodiments, the pharmaceutical preparation as disclosed herein, the pharmaceutical composition as disclosed herein, the pharmaceutical drug substance of as disclosed, or the pharmaceutical drug product as disclosed herein is in parenteral nucleic acid delivery system. The parental nucleic acid delivery system may include the pharmaceutical preparation as disclosed herein, the pharmaceutical composition as disclosed herein, the pharmaceutical drug substance of as disclosed, or the pharmaceutical drug product as disclosed herein, and a parenterally acceptable diluent. In some embodiments, the pharmaceutical preparation as disclosed herein, the pharmaceutical composition as disclosed herein, the pharmaceutical drug substance of as disclosed, or the pharmaceutical drug product as disclosed herein in the parenteral nucleic acid delivery system is free of any carrier.

The disclosure is further directed to a host or host cell including the circular polyribonucleotide described herein. In some embodiments, the host or host cell is a vertebrate, mammal (e.g., human), or other organism or cell.

In some embodiments, the circular polyribonucleotide has a decreased, or fails to produce a, undesired response by the host's immune system as compared to the response triggered by a reference compound, e.g., a linear polynucleotide corresponding to the described circular polyribonucleotide. In embodiments, the circular polyribonucleotide is non-immunogenic in the host. Some immune responses include, but are not limited to, humoral immune responses (e.g., production of immunogen-specific antibodies) and cell-mediated immune responses (e.g., lymphocyte proliferation).

In some embodiments, a host or a host cell is contacted with (e.g., delivered to or administered to) the circular polyribonucleotide. In some embodiments, the host is a mammal, such as a human. The amount of the circular polyribonucleotide or linear, expression product, or both in the host can be measured at any time after administration. In certain embodiments, a time course of host growth in a culture is determined. If the growth is increased or reduced in the presence of the circular polyribonucleotide or linear, the circular polyribonucleotide or expression product or both is identified as being effective in increasing or reducing the growth of the host.

A method of delivering a circular polyribonucleotide molecule as described herein to a cell, tissue, or subject, includes administering the pharmaceutical composition, pharmaceutical drug substance or pharmaceutical drug product as described herein to the cell, tissue, or subject.

In some embodiments, the cell is a eukaryotic cell. In some embodiments, the cell is a mammalian cell. In some embodiments, the cell is an ungulate cell. In some embodiments, the cell is an animal cell. In some embodiments, the cell is an immune cell. In some embodiments, the tissue is a connective tissue, a muscle tissue, a nervous tissue, or an epithelial tissue. In some embodiments, the tissue is an organ (e.g., liver, lung, spleen, kidney, etc.).

In some embodiments, the method of delivering is an in vivo method. For example, a method of delivery of a circular polyribonucleotide as described herein includes parenterally administering to a subject in need thereof, the pharmaceutical composition, pharmaceutical drug substance or pharmaceutical drug product as described herein to the subject in need thereof. As another example, a method of delivering a circular polyribonucleotide to a cell or tissue of a subject, includes administering parenterally to the cell or tissue the pharmaceutical composition, pharmaceutical drug substance or pharmaceutical drug product as described herein. In some embodiments, the circular polyribonucleotide is in an amount effective to elicit a biological response in the subject. In some embodiments, the circular polyribonucleotide is an amount effective to have a biological effect on the cell or tissue in the subject. In some embodiments, the pharmaceutical composition, pharmaceutical drug substance or pharmaceutical drug product as described herein includes a carrier. In some embodiments the pharmaceutical composition, pharmaceutical drug substance or pharmaceutical drug product as described herein includes a diluent and is free of any carrier.

In some embodiments the pharmaceutical composition, the pharmaceutical drug substance, or the pharmaceutical drug product is administered parenterally. In some embodiments the pharmaceutical composition, the pharmaceutical drug substance, or the pharmaceutical drug product is administered intravenously, intraarterially, intraperitoneally, intradermally, intracranially, intrathecally, intralymphaticly, subcutaneously, or intramuscularly. In some embodiments, parenteral administration is intravenously, intramuscularly, ophthalmically, subcutaneously, intradermally or topically.

In some embodiments, the pharmaceutical composition, pharmaceutical drug substance or pharmaceutical drug product as described herein is administered intramuscularly. In some embodiments, the pharmaceutical composition, pharmaceutical drug substance or pharmaceutical drug product as described herein is administered subcutaneously. In some embodiments, the pharmaceutical composition, pharmaceutical drug substance or pharmaceutical drug product as described herein is administered topically. In some embodiments, the pharmaceutical composition, the pharmaceutical drug substance, or the pharmaceutical drug product is administered intratracheally.

In some embodiments the pharmaceutical composition, pharmaceutical drug substance or pharmaceutical drug product is administered by injection. The administration can be systemic administration or local administration. In some embodiments, any of the methods of delivery as described herein are performed with a carrier. In some embodiments, any methods of delivery as described herein are performed without the aid of a carrier or cell penetrating agent.

In some embodiments, the circular polyribonucleotide or a product translated from the circular polyribonucleotide is detected in the cell, tissue, or subject at least 1 day, at least 2 days, at least 3 days, at least 4 days, or at least 5 days after the administering step. In some embodiments, the presence of the circular polyribonucleotide or a product translated from the circular polyribonucleotide is evaluated in the cell, tissue, or subject before the administering step. In some embodiments, the presence of the circular polyribonucleotide or a product translated from the circular polyribonucleotide is evaluated in the cell, tissue, or subject after the administering step.

### Formulations

In some embodiments of the present disclosure a polyribonucleotide (e.g., a circular polyribonucleotide) or a preparation thereof prepared by the methods described herein may be formulated in composition, e.g., a composition for delivery to a cell, a plant, an invertebrate animal, a non-human vertebrate animal, or a human subject, e.g., an agricultural, veterinary, or pharmaceutical composition. In some embodiments, the polyribonucleotide is formulated in a pharmaceutical composition. In some embodiments, a composition includes a polyribonucleotide and a diluent, a carrier, an adjuvant, or a combination thereof. In a particular embodiment, a composition includes a polyribonucleotide described herein and a carrier or a diluent free of any carrier. In some embodiments, a composition including a polyribonucleotide with a diluent free of any carrier is used for naked delivery of the polyribonucleotide (e.g., circular polyribonucleotide) to a subject.

Pharmaceutical compositions may optionally include one or more additional active substances, e.g., therapeutically and/or prophylactically active substances. Pharmaceutical compositions may optionally include an inactive substance that serves as a vehicle or medium for the compositions described herein (e.g., compositions including circular polyribonucleotides, such as any one of the inactive ingredients approved by the United States Food and Drug Administration (FDA) and listed in the Inactive Ingredient Database). Pharmaceutical compositions of the present invention may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference). Non-limiting examples of an inactive substance include solvents, aqueous solvents, non-aqueous solvents, dispersion media, diluents, dispersions, suspension aids, surface active agents, isotonic agents, thickening agents, emulsifying agents, preservatives, polymers, peptides, proteins, cells, hyaluronidases, dispersing agents, granulating agents, disintegrating agents, binding agents, buffering agents (e.g., phosphate buffered saline (PBS)), lubricating agents, oils, and mixtures thereof.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g., non-human mammals. Modification of pharmaceutical compositions suitable for administration to humans to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

In some embodiments, the reference criterion for the amount of linear polyribonucleotide molecules present in the preparation is the presence of no more than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 600 ng/ml, 1 μg/ml, 10 μg/ml, 50 ag/ml, 100 μg/ml, 200 g/ml, 300 μg/ml, 400 μg/ml, 500 μg/ml, 600 μg/ml, 700 μg/ml, 800 μg/ml, 900 μg/ml, 1 mg/ml, 1.5 mg/ml, or 2 mg/ml of linear polyribonucleotide molecules.

In some embodiments, the reference criterion for the amount of circular polyribonucleotide molecules present in the preparation is at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), 99% (w/w), 99.1% (w/w), 99.2% (w/w), 99.3% (w/w), 99.4% (w/w), 99.5% (w/w), 99.6% (w/w), 99.7% (w/w), 99.8% (w/w), 99.9% (w/w), or 100% (w/w) molecules of the total ribonucleotide molecules in the pharmaceutical preparation.

In some embodiments, the reference criterion for the amount of linear polyribonucleotide molecules present in the preparation is no more than 0.5% (w/w), 1% (w/w), 2% (w/w), 5% (w/w), 10% (w/w), 15% (w/w), 20% (w/w), 25% (w/w), 30% (w/w), 40% (w/w), 50% (w/w) linear polyribonucleotide molecules of the total ribonucleotide molecules in the pharmaceutical preparation.

In some embodiments, the reference criterion for the amount of nicked polyribonucleotide molecules present in the preparation is no more than 0.5% (w/w), 1% (w/w), 2% (w/w), 5% (w/w), 10% (w/w), or 15% (w/w) nicked polyribonucleotide molecules of the total ribonucleotide molecules in the pharmaceutical preparation.

In some embodiments, the reference criterion for the amount of combined nicked and linear polyribonucleotide molecules present in the preparation is no more than 0.5% (w/w), 1% (w/w), 2% (w/w), 5% (w/w), 10% (w/w), 15% (w/w), 20% (w/w), 25% (w/w), 30% (w/w), 40% (w/w), 50% (w/w) combined nicked and linear polyribonucleotide molecules of the total ribonucleotide molecules in the pharmaceutical preparation. In some embodiments, a pharmaceutical preparation is an intermediate pharmaceutical preparation of a final circular polyribonucleotide drug product. In some embodiments, a pharmaceutical preparation is a drug substance or active pharmaceutical ingredient (API). In some embodiments, a pharmaceutical preparation is a drug product for administration to a subject.

In some embodiments, a preparation of circular polyribonucleotides is (before, during or after the reduction of linear RNA) further processed to substantially remove DNA, protein contamination (e.g., cell protein such as a host cell protein or protein process impurities), endotoxin, mononucleotide molecules, and/or a process-related impurity.

In some embodiments, a pharmaceutical formulation disclosed herein can include: (i) a compound (e.g., circular polyribonucleotide) disclosed herein; (ii) a buffer; (iii) a non-ionic detergent; (iv) a tonicity agent; and/or (v) a stabilizer. In some embodiments, the pharmaceutical formulation disclosed herein is a stable liquid pharmaceutical formulation. In some embodiments, the pharmaceutical formulation disclosed herein includes protamine or a protamine salt (e.g., protamine sulfate).

The disclosure provides immunogenic compositions including a circular polyribonucleotide described herein. Immunogenic compositions of the disclosure may include a diluent or a carrier, adjuvant, or any combination thereof. Immunogenic compositions of the disclosure may also include one or more immunoregulatory agents, e.g., one or more adjuvants. The adjuvants may include a TH1 adjuvant and/or a TH2 adjuvant, further discussed below. In some embodiments, the immunogenic composition includes a diluent free of any carrier and is used for naked delivery of the circular polyribonucleotide to a subject.

Immunogenic compositions of the disclosure are used to raise an immune response in a subject. The immune response is preferably protective and preferably involves an antibody response (usually including IgG) and/or a cell-mediated immune response. For example, a subject is immunized with an immunogenic composition including a circular polyribonucleotide of the disclosure to induce an immune response. In another example, a subject is immunized with an immunogenic composition including a linear polyribonucleotide including an immunogen to stimulate production of antibodies that bind to the immunogen. By raising an immune response in the subject by these uses and methods, the subject can be protected against various diseases and/or infections e.g., against bacterial and/or viral diseases as discussed above. In certain embodiments, the immunogenic compositions are vaccine compositions. Vaccines according to the disclosure may either be prophylactic (i.e., to prevent infection) or therapeutic (i.e., to treat infection) but will typically be prophylactic. In some embodiments, the subject is a mammal. In some embodiments, the subject is an animal, preferably a mammal, e.g., a human. In one embodiment, the subject is a human. In other embodiments the subject is a non-human mammal, e.g., selected from a cow (e.g., dairy and beef cattle), a sheep, a goat, a pig, a horse, a dog, or a cat. In other embodiments the subject is a bird, e.g., a hen or rooster, turkey, parrot. In some embodiments, the animal is not a mouse or a rabbit or a cow. In a particular embodiment, where the immunogenic composition is for prophylactic use, the human is a child (e.g., a toddler or infant) or a teenager. In another embodiment, where the immunogenic composition is for therapeutic use, the human is a teenager or an adult. An immunogenic composition intended for children may also be administered to adults e.g., to assess safety, dosage, immunogenicity, etc.

Immunogenic composition prepared according to the disclosure may be used to treat both children and adults. A human subject may be less than 1 year old, less than 5 years old, 1-5 years old, 5-15 years old, 15-55 years old, or at least 55 years old. In a particular embodiment, human subjects for receiving the immunogenic compositions are the elderly (e.g., >50 years old, >60 years old, and >65 years), the young (e.g., 55 years old), hospitalized patients, healthcare workers, armed service and military personnel, pregnant women, the chronically ill, or immunodeficient patients. The immunogenic compositions are not suitable solely for these groups, however, and may be used more generally in a population.

In some embodiments, the subject is further immunized with an adjuvant. In some embodiments the subject is further immunized with a vaccine.

### Preservatives

A composition or pharmaceutical composition provided herein can include material for a single administration, or can include material for multiple administrations (e.g., a “multidose” kit). The polyribonucleotide can be present in either linear or circular form. The composition or pharmaceutical composition can include one or more preservatives such as thiomersal or 2-phenoxyethanol. Preservatives can be used to prevent microbial contamination during use. Suitable preservatives include: benzalkonium chloride, thimerosal, chlorobutanol, methyl paraben, propyl paraben, phenylethyl alcohol, edetate disodium, sorbic acid, Onamer M, or other agents known to those skilled in the art. In ophthalmic products, e.g., such preservatives can be employed at a level of from 0.004% to 0.02%. In the compositions described herein the preservative, e.g., benzalkonium chloride, can be employed at a level of from 0.001% to less than 0.01%, e.g., from 0.001% to 0.008%, preferably about 0.005% by weight.

Polyribonucleotides can be susceptible to RNase that can be abundant in ambient environment. Compositions provided herein can include reagents that inhibit RNase activity, thereby preserving the polyribonucleotide from degradation. In some cases, the composition or pharmaceutical composition includes any RNase inhibitor known to one skilled in the art. Alternatively or additionally, the polyribonucleotide, and cell-penetrating agent and/or pharmaceutically acceptable diluents or carriers, vehicles, excipients, or other reagents in the composition provided herein can be prepared in RNase-free environment. The composition can be formulated in RNase-free environment.

In some cases, a composition provided herein can be sterile. The composition can be formulated as a sterile solution or suspension, in suitable vehicles, known in the art. The composition can be sterilized by conventional, known sterilization techniques, e.g., the composition can be sterile filtered.

### Salts

In some cases, a composition or pharmaceutical composition provided herein includes one or more salts. For controlling the tonicity, a physiological salt such as sodium salt can be included in a composition provided herein. Other salts can include potassium chloride, potassium dihydrogen phosphate, disodium phosphate, and/or magnesium chloride, or the like. In some cases, the composition is formulated with one or more pharmaceutically acceptable salts. The one or more pharmaceutically acceptable salts can include those of the inorganic ions, such as, for example, sodium, potassium, calcium, magnesium ions, and the like. Such salts can include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid, or maleic acid. The polyribonucleotide can be present in either linear or circular form.

### Buffers/pH

A composition or pharmaceutical composition provided herein can include one or more buffers, such as a Tris buffer; a borate buffer; a succinate buffer; a histidine buffer (e.g., with an aluminum hydroxide adjuvant); or a citrate buffer. Buffers, in some cases, are included in the 5-20 mM range.

A composition or pharmaceutical composition provided herein can have a pH between about 5.0 and about 8.5, between about 6.0 and about 8.0, between about 6.5 and about 7.5, or between about 7.0 and about 7.8. The composition or pharmaceutical composition can have a pH of about 7. The polyribonucleotide can be present in either linear or circular form.

### Detergents/Surfactants

A composition or pharmaceutical composition provided herein can include one or more detergents and/or surfactants, depending on the intended administration route, e.g., polyoxyethylene sorbitan esters surfactants (commonly referred to as “Tweens”), e.g., polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, e.g., octoxynol-9 (Triton X-100, or t-octylphenoxypolyethoxyethanol); (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as “SPANs”), such as sorbitan trioleate (Span 85) and sorbitan monolaurate, an octoxynol (such as octoxynol-9 (Triton X-100) or t-octylphenoxypolyethoxyethanol), a cetyl trimethyl ammonium bromide (“CTAB”), or sodium deoxycholate. The one or more detergents and/or surfactants can be present only at trace amounts. In some cases, the composition can include less than 1 mg/ml of each of octoxynol-10 and polysorbate 80. Non-ionic surfactants can be used herein. Surfactants can be classified by their “HLB” (hydrophile/lipophile balance). In some cases, surfactants have a HLB of at least 10, at least 15, and/or at least 16. The polyribonucleotide can be present in either linear or circular form.

### Diluents

In some embodiments, an immunogenic composition of the disclosure includes a circular polyribonucleotide and a diluent.

A diluent can be a non-carrier excipient. A non-carrier excipient serves as a vehicle or medium for a composition, such as a circular polyribonucleotide as described herein. Non-limiting examples of a non-carrier excipient include solvents, aqueous solvents, non-aqueous solvents, dispersion media, diluents, dispersions, suspension aids, surface active agents, isotonic agents, thickening agents, emulsifying agents, preservatives, polymers, peptides, proteins, cells, hyaluronidases, dispersing agents, granulating agents, disintegrating agents, binding agents, buffering agents (e.g., phosphate buffered saline (PBS)), lubricating agents, oils, and mixtures thereof. A non-carrier excipient can be any one of the inactive ingredients approved by the United States Food and Drug Administration (FDA) and listed in the Inactive Ingredient Database that does not exhibit a cell-penetrating effect. A non-carrier excipient can be any inactive ingredient suitable for administration to a non-human animal, for example, suitable for veterinary use. Modification of compositions suitable for administration to humans in order to render the compositions suitable for administration to various animals is well understood, and the ordinarily skilled veterinary pharmacologist can design and/or perform such modification with merely ordinary, if any, experimentation.

In some embodiments, the circular polyribonucleotide may be delivered as a naked delivery formulation, such as including a diluent. A naked delivery formulation delivers a circular polyribonucleotide, to a cell without the aid of a carrier and without modification or partial or complete encapsulation of the circular polyribonucleotide, capped polyribonucleotide, or complex thereof.

A naked delivery formulation is a formulation that is free from a carrier and wherein the circular polyribonucleotide is without a covalent modification that binds a moiety that aids in delivery to a cell or without partial or complete encapsulation of the circular polyribonucleotide. In some embodiments, a circular polyribonucleotide without a covalent modification that binds a moiety that aids in delivery to a cell is a polyribonucleotide that is not covalently bound to a protein, small molecule, a particle, a polymer, or a biopolymer. A circular polyribonucleotide without covalent modification that binds a moiety that aids in delivery to a cell does not contain a modified phosphate group. For example, a circular polyribonucleotide without a covalent modification that binds a moiety that aids in delivery to a cell does not contain phosphorothioate, phosphoroselenates, boranophosphates, boranophosphate esters, hydrogen phosphonates, phosphoramidates, phosphorodiamidates, alkyl or aryl phosphonates, or phosphotriesters.

In some embodiments, a naked delivery formulation is free of any or all of: transfection reagents, cationic carriers, carbohydrate carriers, nanoparticle carriers, or protein carriers. In some embodiments, a naked delivery formulation is free from phytoglycogen octenyl succinate, phytoglycogen beta-dextrin, anhydride-modified phytoglycogen beta-dextrin, lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane (DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-l-propanaminium trifluoroacetate (DOSPA), 3B-[N-(N\N′-Dimethylaminoethane)-carbamoyl]Cholesterol Hydrochloride (DC-Cholesterol HCl), diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride (DODAC), human serum albumin (HSA), low-density lipoprotein (LDL), high-density lipoprotein (HDL), or globulin.

In certain embodiments, a naked delivery formulation includes a non-carrier excipient. In some embodiments, a non-carrier excipient includes an inactive ingredient that does not exhibit a cell-penetrating effect. In some embodiments, a non-carrier excipient includes a buffer, for example PBS. In some embodiments, a non-carrier excipient is a solvent, a non-aqueous solvent, a diluent, a suspension aid, a surface-active agent, an isotonic agent, a thickening agent, an emulsifying agent, a preservative, a polymer, a peptide, a protein, a cell, a hyaluronidase, a dispersing agent, a granulating agent, a disintegrating agent, a binding agent, a buffering agent, a lubricating agent, or an oil.

In some embodiments, a naked delivery formulation includes a diluent. A diluent may be a liquid diluent or a solid diluent. In some embodiments, a diluent is an RNA solubilizing agent, a buffer, or an isotonic agent. Examples of an RNA solubilizing agent include water, ethanol, methanol, acetone, formamide, and 2-propanol. Examples of a buffer include 2-(N-morpholino)ethanesulfonic acid (MES), Bis-Tris, 2-[(2-amino-2-oxoethyl)-(carboxymethyl)amino]acetic acid (ADA), N-(2-Acetamido)-2-aminoethanesulfonic acid (ACES), piperazine-N,N′-bis(2-ethanesulfonic acid) (PIPES), 2-[[1,3-dihydroxy-2-(hydroxymethyl)propan-2-yl]amino]ethanesulfonic acid (TES), 3-(N-morpholino)propanesulfonic acid (MOPS), 4-(2-hydroxyethyl)-1-piperazineethanesulfonic acid (HEPES), Tris, Tricine, Gly-Gly, Bicine, or phosphate. Examples of an isotonic agent include glycerin, mannitol, polyethylene glycol, propylene glycol, trehalose, or sucrose.

### Carriers

In some embodiments, an immunogenic composition of the disclosure includes a circular polyribonucleotide and a carrier.

In certain embodiments, an immunogenic composition includes a circular polyribonucleotide as described herein in a vesicle or other membrane-based carrier.

In other embodiments, an immunogenic composition includes the circular polyribonucleotide in or via a cell, vesicle or other membrane-based carrier. In one embodiment, an immunogenic composition includes the circular polyribonucleotide in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral, or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, JOURNAL OF DRUG DELIVERY, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, JOURNAL OF DRUG DELIVERY, vol. 2011, Article ID 469679, 12 pages, 2011. doi:10.1155/2011/469679 for review). Extruded lipids can be prepared by extruding through filters of decreasing size, as described in Templeton et al., NATURE BIOTECH, 15:647-652, 1997, the teachings of which relating to extruded lipid preparation are incorporated herein by reference.

In certain embodiments, an immunogenic composition of the disclosure includes a circular polyribonucleotide and lipid nanoparticles, for example lipid nanoparticles described herein. Lipid nanoparticles are another example of a carrier that provides a biocompatible and biodegradable delivery system for a circular polyribonucleotide molecule as described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Additional non-limiting examples of carriers include carbohydrate carriers (e.g., an anhydride-modified phytoglycogen or glycogen-type material), protein carriers (e.g., a protein covalently linked to the circular polyribonucleotide), or cationic carriers (e.g., a cationic lipopolymer or transfection reagent). Non-limiting examples of carbohydrate carriers include phtoglycogen octenyl succinate, phytoglycogen beta-dextrin, and anhydride-modified phytoglycogen beta-dextrin. Non-limiting examples of cationic carriers include lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane(DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-l-propanaminium trifluoroacetate (DOSPA), 3B-[N-(N\N′-Dimethylaminoethane)-carbamoyl]Cholesterol Hydrochloride (DC-Cholesterol HCl), diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), and N,N-dioleyl-N,N-dimethylammonium chloride (DODAC). Non-limiting examples of protein carriers include human serum albumin (HSA), low-density lipoprotein (LDL), high-density lipoprotein (HDL), or globulin.

Exosomes can also be used as drug delivery vehicles for a circular RNA composition or preparation described herein. For a review, see Ha et al. July 2016. ACTA PHARMACEUTICA SINICA B. Volume 6, Issue 4, Pages 287-296; https://doi.org/10.1016/j.apsb.2016.02.001.

Ex vivo differentiated red blood cells can also be used as a carrier for a circular RNA composition or preparation described herein. See, e.g., International Patent Publication Nos. WO2015/073587; WO2017/123646; WO2017/123644; WO2018/102740; WO2016/183482; WO2015/153102; WO2018/151829; WO2018/009838; Shi et al. 2014. PROC NATL ACAD SCI USA. 111(28): 10131-136; U.S. Pat. No. 9,644,180; Huang et al. 2017. NATURE COMMUNICATIONS 8: 423; Shi et al. 2014. PROC NATL ACAD SCI USA. 111(28): 10131-136.

Fusosome compositions, e.g., as described in International Patent Publication No. WO2018/208728, can also be used as carriers to deliver a circular polyribonucleotide molecule described herein.

Virosomes and virus-like particles (VLPs) can also be used as carriers to deliver a circular polyribonucleotide molecule described herein to targeted cells.

Plant nanovesicles and plant messenger packs (PMPs), e.g., as described in International Patent Publication Nos. WO2011/097480, WO2013/070324, WO2017/004526, or WO2020/041784 can also be used as carriers to deliver the circular RNA composition or preparation described herein.

Microbubbles can also be used as carriers to deliver a circular polyribonucleotide molecule described herein. See, e.g., U.S. Pat. No. 7,115,583; Beeri, R. et al., CIRCULATION. 2002 Oct. 1; 106(14):1756-59; Bez, M. et al., NAT PROTOC. 2019 April; 14(4): 1015-26; Hernot, S. et al., ADV DRUG DELIV REV. 2008 Jun. 30; 60(10): 1153-66; Rychak, J. J. et al., ADV DRUG DELIV REV. 2014 June; 72: 82-93. In some embodiments, microbubbles are albumin-coated perfluorocarbon microbubbles.

The carrier including the circular polyribonucleotides described herein may include a plurality of particles. The particles may have median article size of 30 to 700 nanometers (e.g., 30 to 50, 50 to 100, 100 to 200, 200 to 300, 300 to 400, 400 to 500, 500 to 600, 600 to 700, 100 to 500, 50 to 500, or 200 to 700 nanometers). The size of the particle may be optimized to favor deposition of the payload, including the circular polyribonucleotide into a cell. Deposition of the circular polyribonucleotide into certain cell types may favor different particle sizes. For example, the particle size may be optimized for deposition of the circular polyribonucleotide into antigen presenting cells. The particle size may be optimized for deposition of the circular polyribonucleotide into dendritic cells. Additionally, the particle size may be optimized for depositions of the circular polyribonucleotide into draining lymph node cells.

### Lipid Nanoparticles

The compositions, methods, and delivery systems provided by the present disclosure may employ any suitable carrier or delivery modality described herein, including, in certain embodiments, lipid nanoparticles (LNPs). Lipid nanoparticles, in some embodiments, include one or more ionic lipids, such as non-cationic lipids (e.g., neutral or anionic, or zwitterionic lipids); one or more conjugated lipids (such as PEG-conjugated lipids or lipids conjugated to polymers described in Table 5 of WO2019217941; incorporated herein by reference in its entirety); one or more sterols (e.g., cholesterol).

Lipids that can be used in nanoparticle formations (e.g., lipid nanoparticles) include, for example those described in Table 4 of WO2019217941, which is incorporated by reference—e.g., a lipid-containing nanoparticle can include one or more of the lipids in Table 4 of WO2019217941. Lipid nanoparticles can include additional elements, such as polymers, such as the polymers described in Table 5 of WO2019217941, incorporated by reference.

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-1-O-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), and combinations of the foregoing.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al. (2020), dx.doi.org/10.1021/acs.nanolett.0c01386, incorporated herein by reference.

In some embodiments, the lipid particle includes an ionizable lipid, a non-cationic lipid, a conjugated lipid that inhibits aggregation of particles, and a sterol. The amounts of these components can be varied independently and to achieve desired properties. For example, in some embodiments, the lipid nanoparticle includes an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid can be varied as desired. For example, the total lipid to nucleic acid (mass or weight) ratio can be from about 10:1 to about 30:1.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Some non-limiting example of lipid compounds that may be used (e.g., in combination with other lipid components) to form lipid nanoparticles for the delivery of compositions described herein, e.g., nucleic acid (e.g., RNA (e.g., circular polyribonucleotide, linear polyribonucleotide)) described herein includes,

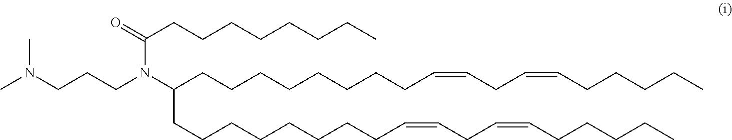 [SMILES: CCCCC/C=C\C/C=C\CCCCCCCCC(CCCCCCCC/C=C\C/C=C\CCCCC)N(CCCN(C)C)C(=O)CCCCCCCC]

In some embodiments an LNP including Formula (i) is used to deliver a polyribonucleotide (e.g., a circular polyribonucleotide, a linear polyribonucleotide) composition described herein to cells.

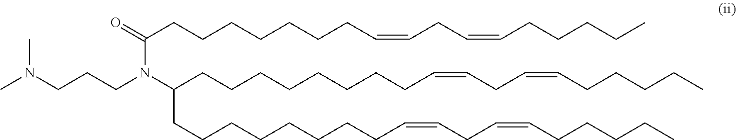 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC]

In some embodiments an LNP including Formula (ii) is used to deliver a polyribonucleotide (e.g., a circular polyribonucleotide, a linear polyribonucleotide) composition described herein to cells.

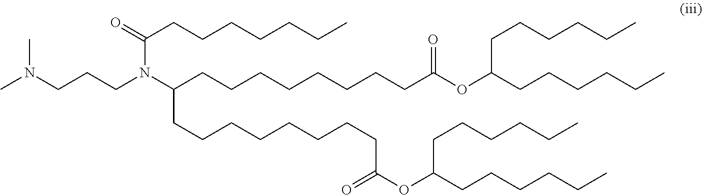 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

In some embodiments an LNP including Formula (iii) is used to deliver a polyribonucleotide (e.g., a circular polyribonucleotide, a linear polyribonucleotide) composition described herein to cells.

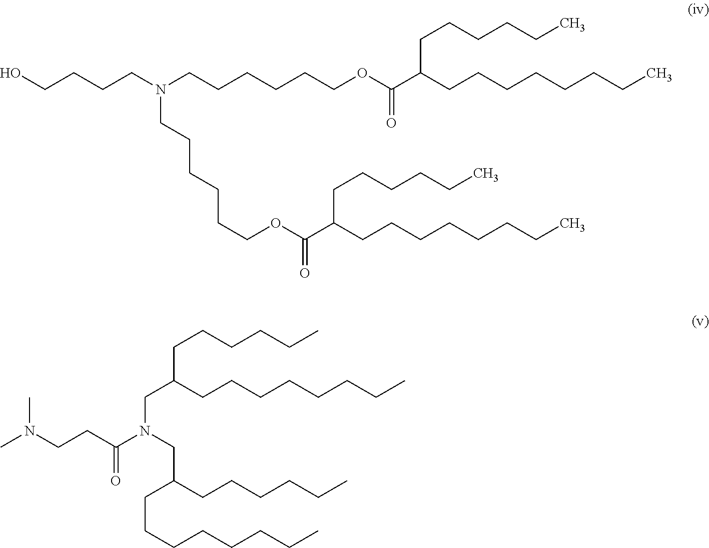

In some embodiments an LNP including Formula (v) is used to deliver a polyribonucleotide (e.g., a circular polyribonucleotide, a linear polyribonucleotide) composition described herein to cells.

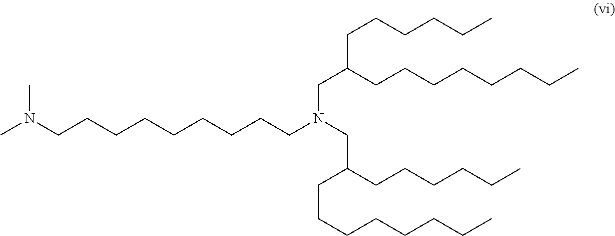 [SMILES: CCCCCCCCC(CCCCCC)CN(CCCCCCCCCN(C)C)CC(CCCCCC)CCCCCCCC]

In some embodiments an LNP including Formula (vi) is used to deliver a polyribonucleotide (e.g., a circular polyribonucleotide, a linear polyribonucleotide) composition described herein to cells.

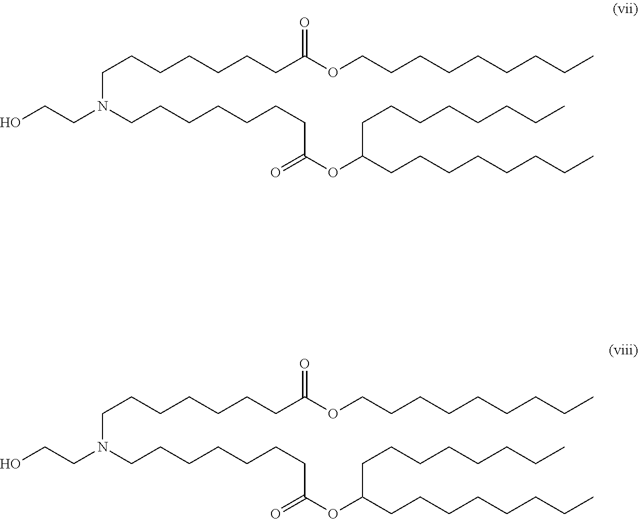 [SMILES: CCCCCCCCCOC(=O)CCCCCCCN(CCO)CCCCCCCC(=O)OC(CCCCCCCC)CCCCCCCC ; CCCCCCCCCOC(=O)CCCCCCCN(CCO)CCCCCCCC(=O)OC(CCCCCCCC)CCCCCCCC]

In some embodiments an LNP including Formula (viii) is used to deliver a polyribonucleotide (e.g., a circular polyribonucleotide, a linear polyribonucleotide) composition described herein to cells.

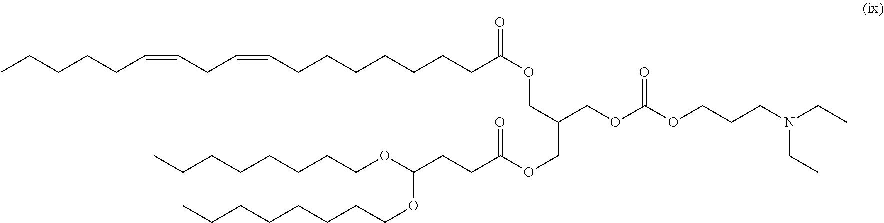 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments an LNP including Formula (ix) is used to deliver a polyribonucleotide (e.g., a circular polyribonucleotide, a linear polyribonucleotide) composition described herein to cells.

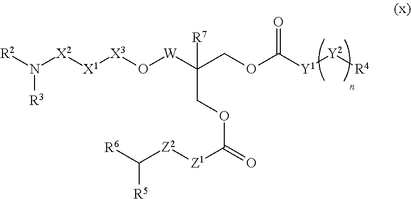

wherein
X¹ is O, NR¹, or a direct bond, X² is C2-5 alkylene, X³ is C(═O) or a direct bond, R¹ is H or Me, R³ is C1-3 alkyl, R² is C1-3 alkyl, or R² taken together with the nitrogen atom to which it is attached and 1-3 carbon atoms of X² form a 4-, 5-, or 6-membered ring, or X¹ is NR¹, R¹ and R² taken together with the nitrogen atoms to which they are attached form a 5- or 6-membered ring, or R² taken together with R³ and the nitrogen atom to which they are attached form a 5-, 6-, or 7-membered ring, Y¹ is C2-12 alkylene, Y² is selected from

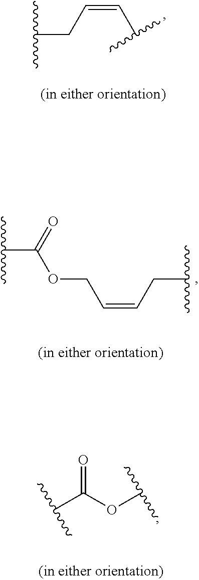

n is 0 to 3, R⁴ is C1-15 alkyl, Z¹ is C1-6 alkylene or a direct bond,
Z² is

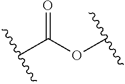 [SMILES: *OC(*)=O]

(in either orientation) or absent, provided that if Z¹ is a direct bond, Z² is absent;
R⁵ is C5-9 alkyl or C6-10 alkoxy, R⁶ is C5-9 alkyl or C6-10 alkoxy, W is methylene or a direct bond, and
R⁷ is H or Me, or a salt thereof, provided that if R³ and R² are C2 alkyls, X¹ is O, X² is linear C3 alkylene,
X³ is C(═O), Y¹ is linear Ce alkylene, (Y²)n-R⁴ is

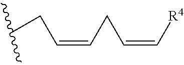

R⁴ is linear C5 alkyl, Z¹ is C2 alkylene, Z² is absent, W is methylene, and R⁷ is H, then R⁵ and R⁶ are not Cx alkoxy.

In some embodiments an LNP including Formula (xii) is used to deliver a polyribonucleotide (e.g., a circular polyribonucleotide, a linear polyribonucleotide) composition described herein to cells.

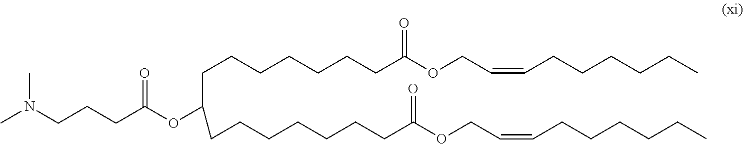 [SMILES: CCCCCC/C=C\COC(=O)CCCCCCCC(CCCCCCCC(=O)OC/C=C\CCCCCC)OC(=O)CCCN(C)C]

In some embodiments an LNP including Formula (xi) is used to deliver a polyribonucleotide (e.g., a circular polyribonucleotide, a linear polyribonucleotide) composition described herein to cells.

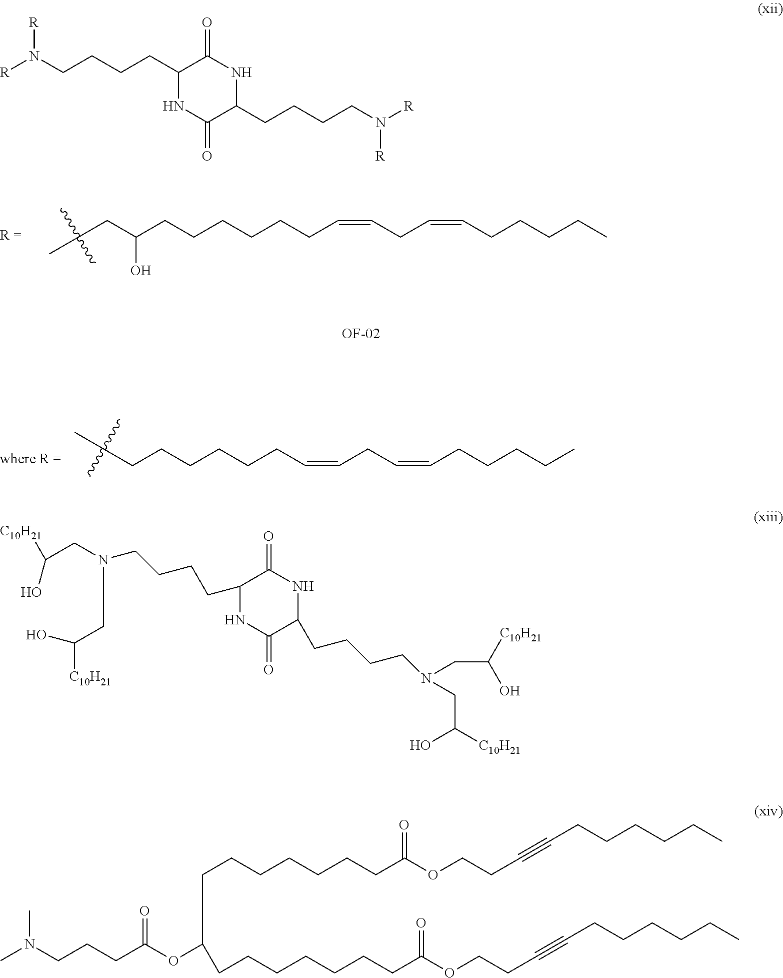

In some embodiments an LNP includes a compound of Formula (xiii) and a compound of Formula (xiv).

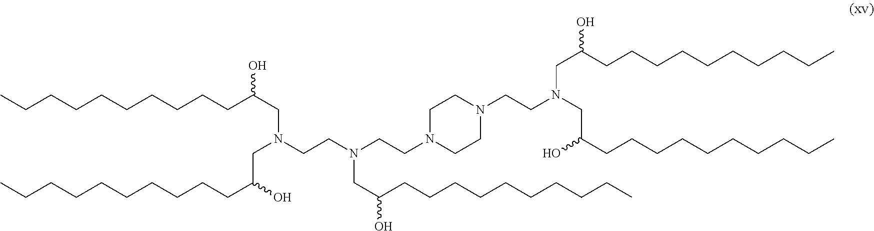 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments an LNP including Formula (xv) is used to deliver a polyribonucleotide (e.g., a circular polyribonucleotide, a linear polyribonucleotide) composition described herein to cells.

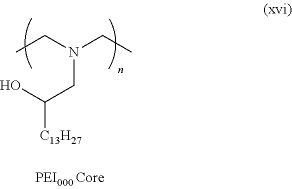

In some embodiments an LNP including a formulation of Formula (xvi) is used to deliver a polyribonucleotide (e.g., a circular polyribonucleotide, a linear polyribonucleotide) composition described herein to cells.

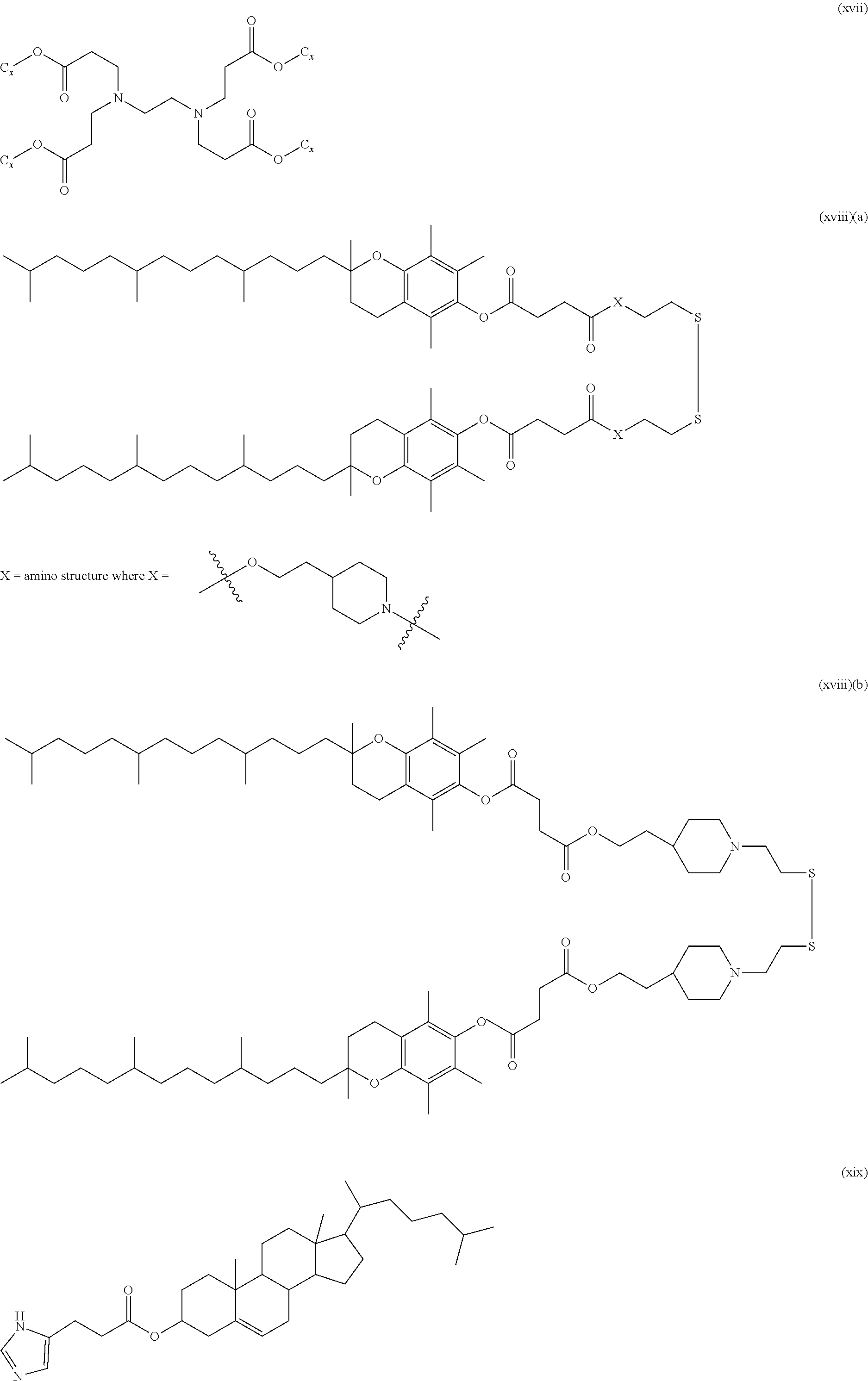

In some embodiments, a lipid compound used to form lipid nanoparticles for the delivery of compositions described herein, e.g., nucleic acid (e.g., RNA (e.g., circular polyribonucleotide, linear polyribonucleotide)) described herein is made by one of the following reactions:

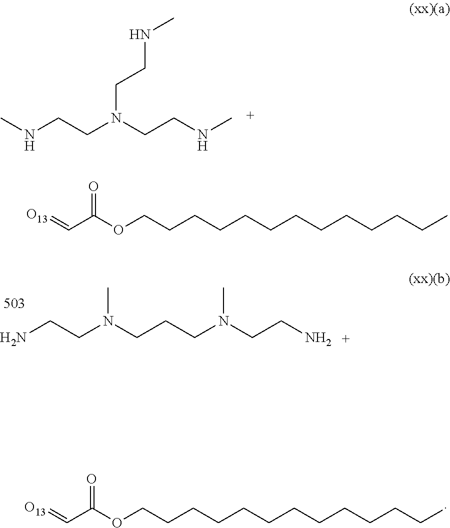

In some embodiments an LNP including Formula (xxi) is used to deliver a polyribonucleotide (e.g., a circular polyribonucleotide, a linear polyribonucleotide) composition described herein to cells. In some embodiments the LNP of Formula (xxi) is an LNP described by WO2021113777 (e.g., a lipid of Formula (1) such as a lipid of Table 1 of WO2021113777).

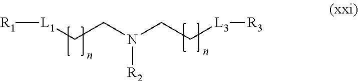

wherein
each n is independently an integer from 2-15; L₁ and L₃ are each independently —OC(O)—* or —C(O)O—*, wherein “*” indicates the attachment point to R₁ or R₃;
R₁ and R₃ are each independently a linear or branched C9-C20 alkyl or C9-C20 alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkyl sulfonyl, and alkyl sulfonealkyl; and
R₂ is selected from a group consisting of:

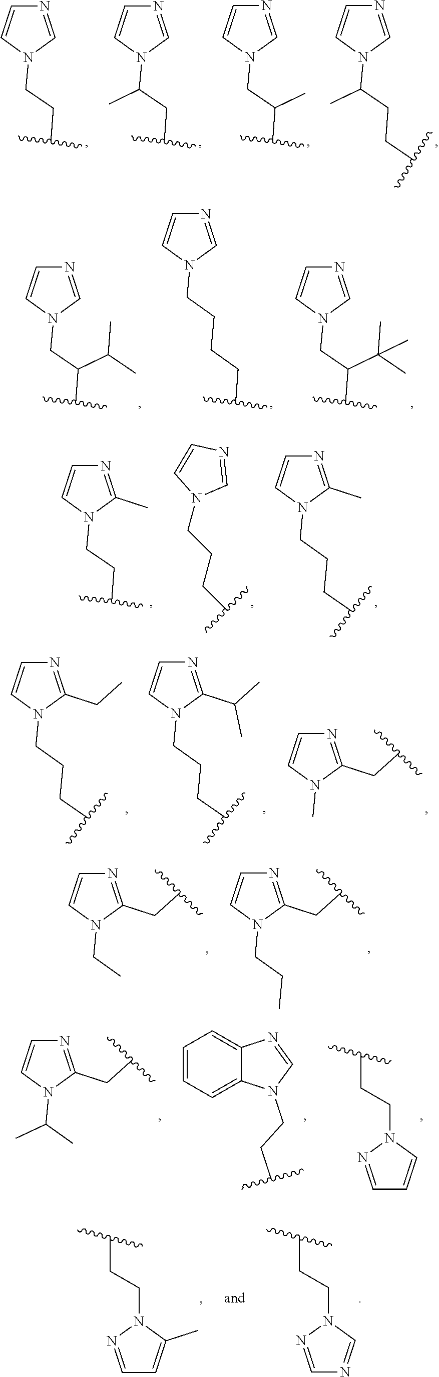

In some embodiments an LNP including Formula (xxii) is used to deliver a polyribonucleotide (e.g., a circular polyribonucleotide, a linear polyribonucleotide) composition described herein to cells. In some embodiments the LNP of Formula (xxii) is an LNP described by WO2021113777 (e.g., a lipid of Formula (2) such as a lipid of Table 2 of WO2021113777).

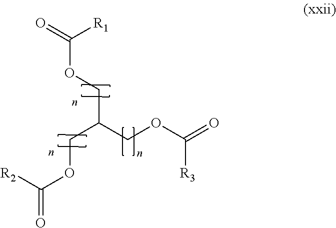

wherein
each n is independently an integer from 1-15;
R₁ and R₂ are each independently selected from a group consisting of:

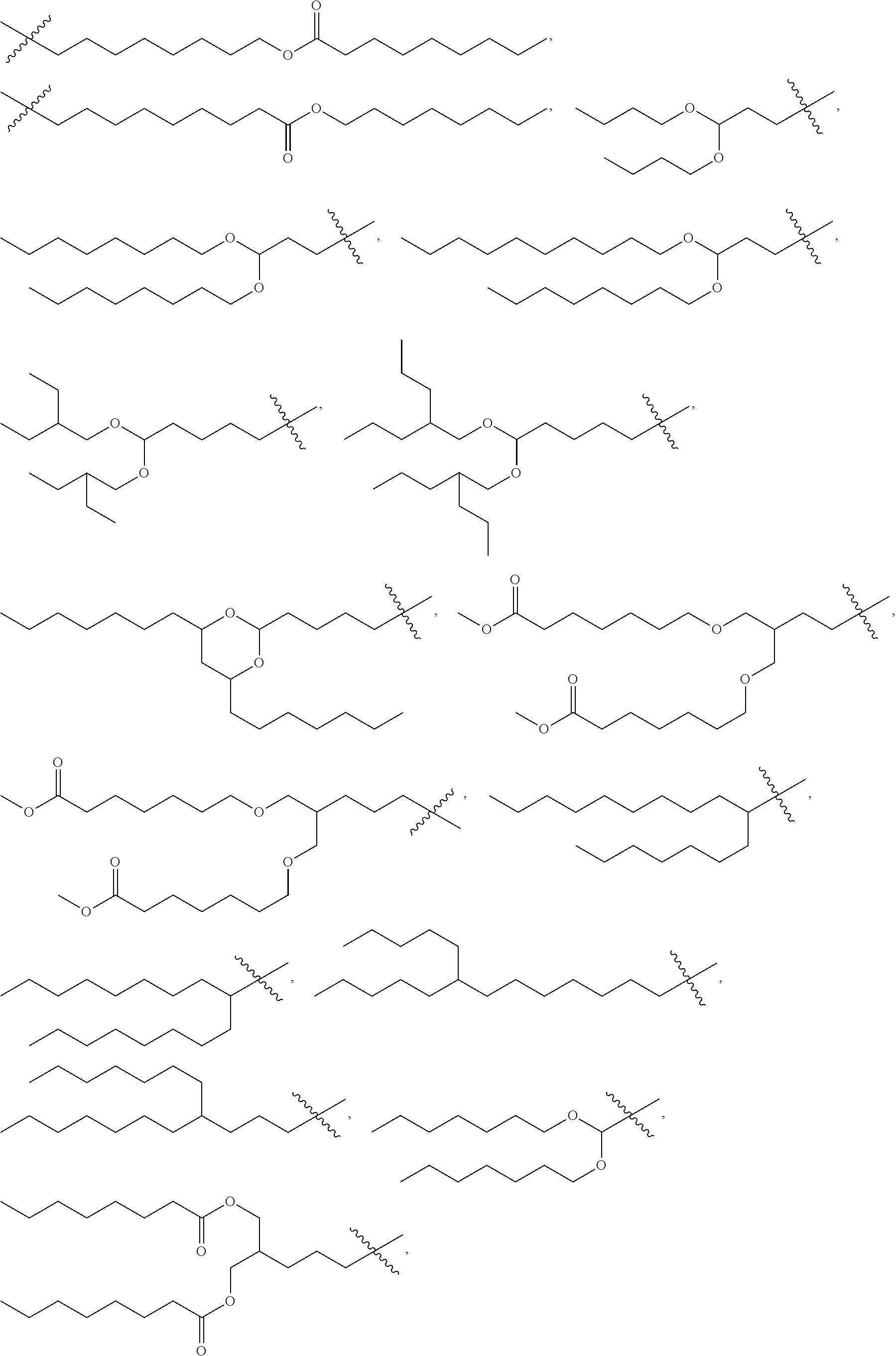

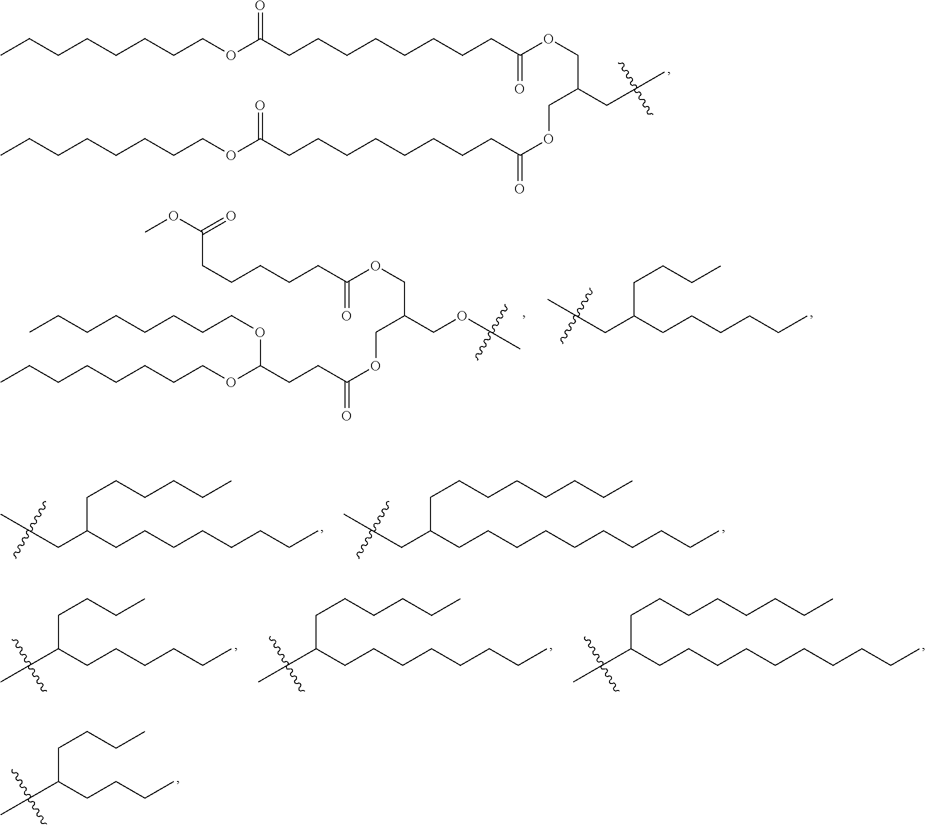

R₃ is selected from a group consisting c 94

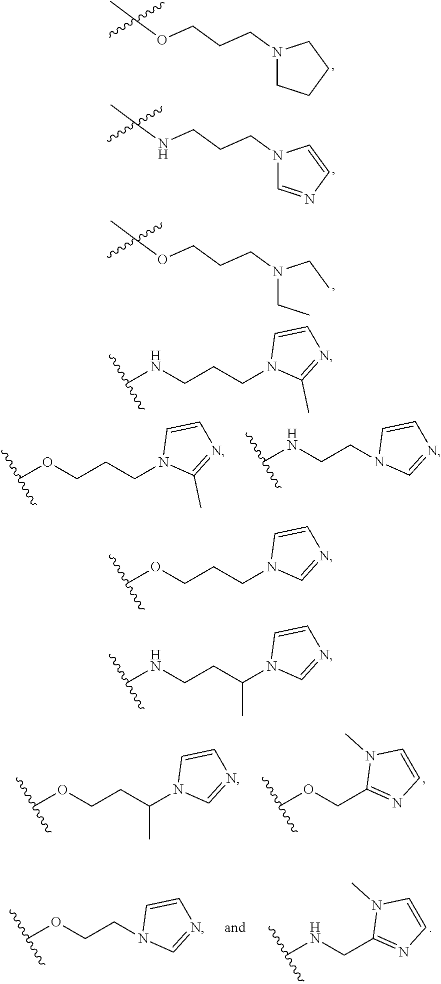

In some embodiments an LNP including Formula (xxiii) is used to deliver a polyribonucleotide (e.g., a circular polyribonucleotide, a linear polyribonucleotide) composition described herein to cells. In some embodiments the LNP of Formula (xxiii) is an LNP described by WO2021113777 (e.g., a lipid of Formula (3) such as a lipid of Table 3 of WO2021113777).

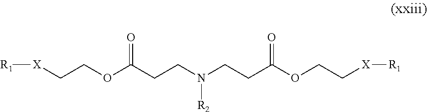

wherein
X is selected from —O—, —S—, or —OC(O)—*, wherein * indicates the attachment point to R₁;
R₁ is selected from a group consisting of:

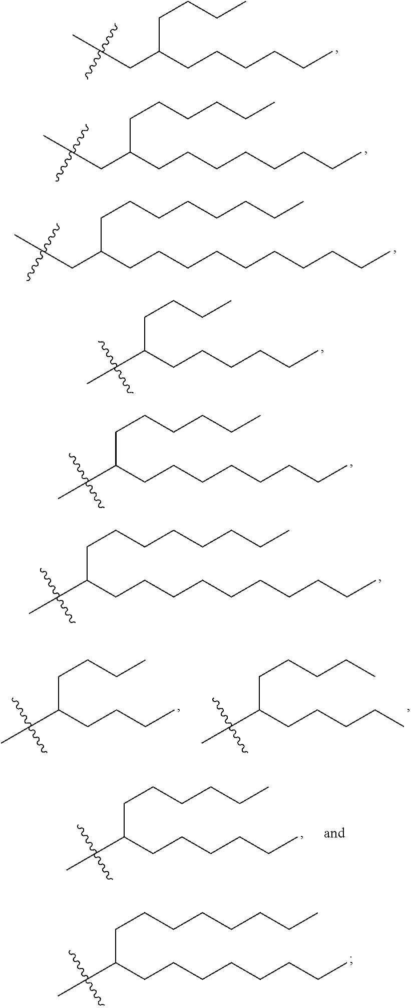

and R₂ is selected from a group consisting of:

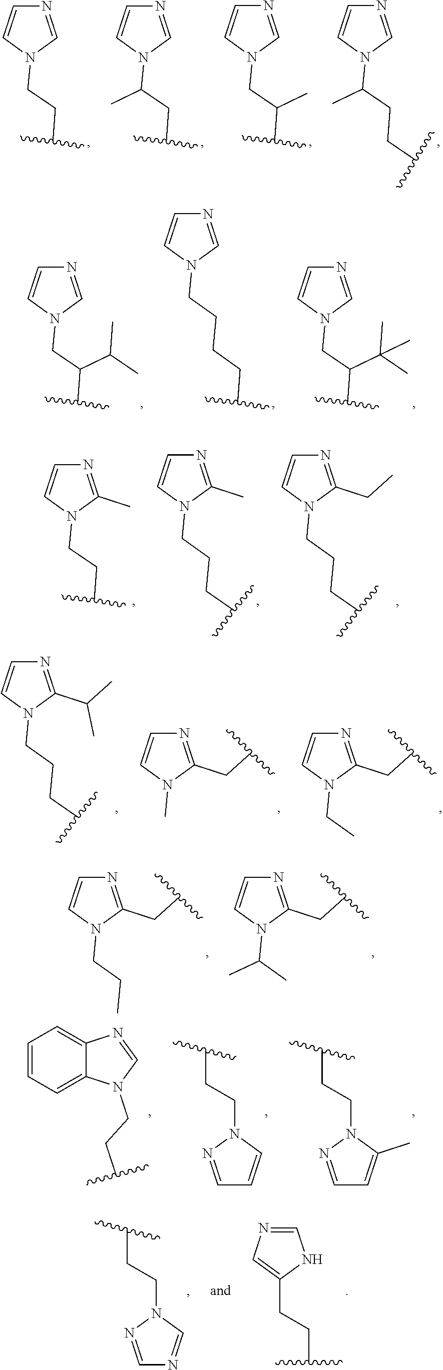

In some embodiments, a composition described herein (e.g., a nucleic acid (e.g., a circular polyribonucleotide, a linear polyribonucleotide) or a protein) is provided in an LNP that includes an ionizable lipid. In some embodiments, the ionizable lipid is heptadecan-9-yl 8-((2-hydroxyethyl)(6-oxo-6-(undecyloxy)hexyl)amino)octanoate (SM-102); e.g., as described in Example 1 of U.S. Pat. No. 9,867,888 (incorporated by reference herein in its entirety). In some embodiments, the ionizable lipid is 9Z,12Z)-3-((4,4-bis(octyloxy)butanoyl)oxy)-2-((((3-(diethylamino)propoxy)carbonyl)oxy)methyl)propyl octadeca-9,12-dienoate (LP01), e.g., as synthesized in Example 13 of WO2015/095340 (incorporated by reference herein in its entirety). In some embodiments, the ionizable lipid is Di((Z)-non-2-en-1-yl) 9-((4-dimethylamino)butanoyl)oxy)heptadecanedioate (L319), e.g., as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl)(2-hydroxydodecyl) amino)ethyl)piperazin-1-yl)ethyl)azanediyl)bis(dodecan-2-ol) (C12-200), e.g., as synthesized in Examples 14 and 16 of WO2010/053572 (incorporated by reference herein in its entirety). In some embodiments, the ionizable lipid is Imidazole cholesterol ester (ICE) lipid (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-IH-cyclopenta[a]phenanthren-3-yl 3-(1H-imidazol-4-yl)propanoate, e.g., Structure (I) from WO2020/106946 (incorporated by reference herein in its entirety).

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle includes a cationic lipid in formulation with one or more of neutral lipids, ionizable amine-containing lipids, biodegradable alkyne lipids, steroids, phospholipids including polyunsaturated lipids, structural lipids (e.g., sterols), PEG, cholesterol, and polymer conjugated lipids. In some embodiments, the cationic lipid may be an ionizable cationic lipid. An exemplary cationic lipid as disclosed herein may have an effective pKa over 6.0. In embodiments, a lipid nanoparticle may include a second cationic lipid having a different effective pKa (e.g., greater than the first effective pKa), than the first cationic lipid. A lipid nanoparticle may include between 40 and 60 mol percent of a cationic lipid, a neutral lipid, a steroid, a polymer conjugated lipid, and a therapeutic agent, e.g., a nucleic acid (e.g., RNA (e.g., a circular polyribonucleotide, a linear polyribonucleotide)) described herein, encapsulated within or associated with the lipid nanoparticle. In some embodiments, the nucleic acid is co-formulated with the cationic lipid. The nucleic acid may be adsorbed to the surface of an LNP, e.g., an LNP including a cationic lipid. In some embodiments, the nucleic acid may be encapsulated in an LNP, e.g., an LNP including a cationic lipid. In some embodiments, the lipid nanoparticle may include a targeting moiety, e.g., coated with a targeting agent. In embodiments, the LNP formulation is biodegradable. In some embodiments, a lipid nanoparticle including one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of an RNA molecule.

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or III of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; I, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, or III of US2013/0116307; I, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; III-3 of WO2018/081480; I-5 or I-8 of WO2020/081938; 18 or 25 of U.S. Pat. No. 9,867,888; A of US2019/0136231; II of WO2020/219876; I of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-013 or 503-013 of Whitehead et al; TS-P4C2 of U.S. Pat. No. 9,708,628; I of WO2020/106946; I of WO2020/106946; and (1), (2), (3), or (4) of WO2021/113777. Exemplary lipids further include a lipid of any one of Tables 1-16 of WO2021/113777.

In some embodiments, the ionizable lipid is MC3 (6Z,9Z,28Z,3 IZ)-heptatriaconta-6,9,28,3 I-tetraen-I9-yl-4-(dimethylamino) butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (incorporated by reference herein in its entirety). In some embodiments, the ionizable lipid is (13Z,16Z)-A,A-dimethyl-3-nonyldocosa-13, 16-dien-1-amine (Compound 32), e.g., as described in Example 11 of WO2019051289A9 (incorporated by reference herein in its entirety). In some embodiments, the ionizable lipid is Compound 6 or Compound 22, e.g., as described in Example 12 of WO2019051289A9 (incorporated by reference herein in its entirety).

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), monomethyl-phosphatidylethanolamine (such as 16-O-monomethyl PE), dimethyl-phosphatidylethanolamine (such as 16-O-dimethyl PE), I8-I-trans PE, I-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, palmitoyl, stearoyl, or oleoyl. Additional exemplary lipids, in certain embodiments, include, without limitation, those described in Kim et al. (2020) dx.doi.org/10.1021/acs.nanolett.0c01386, incorporated herein by reference. Such lipids include, in some embodiments, plant lipids found to improve liver transfection with mRNA (e.g., DGTS).

Other examples of non-cationic lipids suitable for use in the lipid nanoparticles include, without limitation, nonphosphorous lipids such as, e.g., stearylamine, dodeeylamine, hexadecylamine, acetyl palmitate, glycerol ricinoleate, hexadecyl stereate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the contents of which is incorporated herein by reference in their entirety.

In some embodiments, the non-cationic lipid is oleic acid or a compound of Formula I, II, or IV of US2018/0028664, incorporated herein by reference in its entirety. The non-cationic lipid can include, for example, 0-30% (mol) of the total lipid present in the lipid nanoparticle. In some embodiments, the non-cationic lipid content is 5-20% (mol) or 10-15% (mol) of the total lipid present in the lipid nanoparticle. In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not include any phospholipids.

In some aspects, the lipid nanoparticle can further include a component, such as a sterol, to provide membrane integrity. One exemplary sterol that can be used in the lipid nanoparticle is cholesterol and derivatives thereof. Non-limiting examples of cholesterol derivatives include polar analogues such as 5a-cholestanol, 53-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., cholesteryl-(4′-hydroxy)-butyl ether. Exemplary cholesterol derivatives are described in PCT publication WO2009/127060 and US patent publication US2010/0130588, each of which is incorporated herein by reference in its entirety.

In some embodiments, the component providing membrane integrity, such as a sterol, can include 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can include a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-1-O-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, and US2018/0028664, and WO2017/099823, the contents of all of which are incorporated herein by reference in their entirety. In some embodiments, a PEG-lipid is a compound of Formula III, III-a-1, III-a-2, Ill-b-1, Ill-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (I-[8′-(Cholest-5-en-3[beta]-oxy)carboxamido-3′,6′-dioxaoctanyl] carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some embodiments, the PEG-lipid includes PEG-DMG, 1,2-dimyristoyl-sn-glycero-3-phosphoethanolamine-N-[methoxy(polyethylene glycol)-2000]. In some embodiments, the PEG-lipid includes a structure selected from:

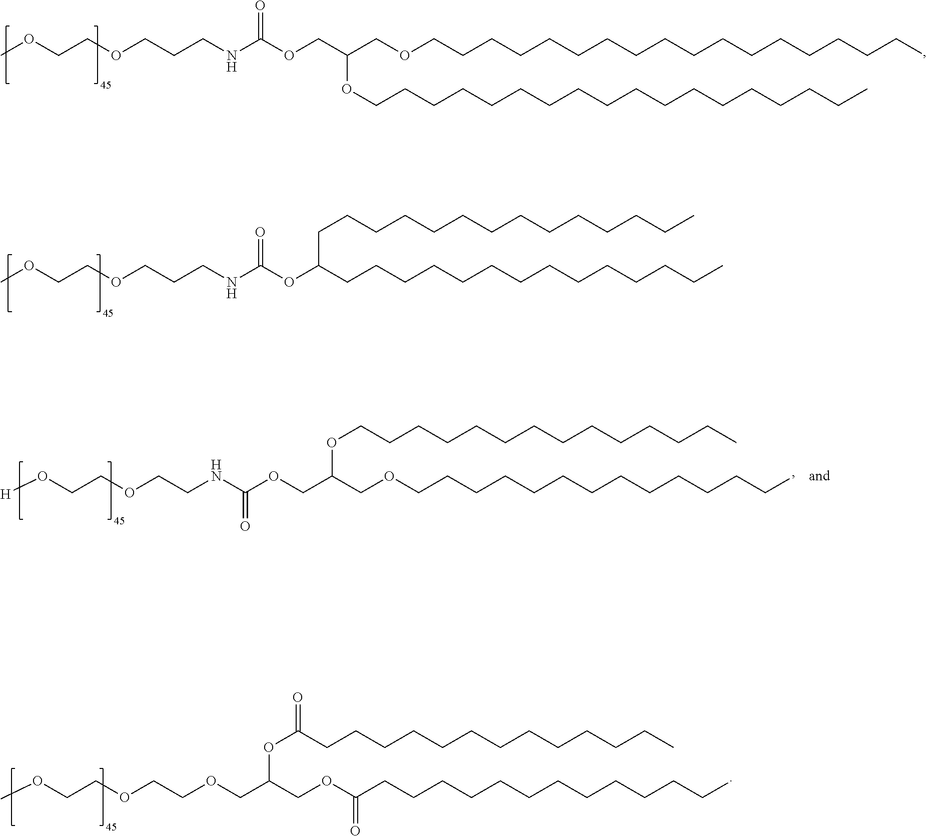

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT, and LIS patent applications listed in Table 2 of WO2019051289A9, the contents of all of which are incorporated herein by reference in their entirety.

In some embodiments, the PEG or the conjugated lipid can include 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can include 30-70% ionizable lipid by mole or by total weight of the composition, 0-60% cholesterol by mole or by total weight of the composition, 0-30% non-cationic lipid by mole or by total weight of the composition and 1-10% conjugated lipid by mole or by total weight of the composition. Preferably, the composition includes 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example including 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation includes ionizable lipid, phospholipid, cholesterol and a PEG-ylated lipid in a molar ratio of 50:10:38.5:1.5. In some other embodiments, the lipid particle formulation includes ionizable lipid, cholesterol and a PEG-ylated lipid in a molar ratio of 60:38.5:1.5.

In some embodiments, the lipid particle includes ionizable lipid, non-cationic lipid (e.g., phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle includes ionizable lipid/non-cationic-lipid/sterol/conjugated lipid at a molar ratio of 50:10:38.5:1.5.

In an aspect, the disclosure provides a lipid nanoparticle formulation including phospholipids, lecithin, phosphatidylcholine and phosphatidylethanolamine.

In some embodiments, one or more additional compounds can also be included. Those compounds can be administered separately, or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In some embodiments, the LNPs include biodegradable, ionizable lipids. In some embodiments, the LNPs include (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, the average LNP diameter of the LNP formulation may be between 10s of nm and 100s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about I mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

A LNP may, in some instances, be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of an LNP may be used to indicate the electrokinetic potential of the composition. In some embodiments, the zeta potential may describe the surface charge of an LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a protein and/or nucleic acid describes the amount of protein and/or nucleic acid that is encapsulated or otherwise associated with a LNP after preparation, relative to the initial amount provided. The encapsulation efficiency is desirably high (e.g., close to 100%). The encapsulation efficiency may be measured, for example, by comparing the amount of protein or nucleic acid in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the lipid nanoparticles described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

A LNP may optionally include one or more coatings. In some embodiments, a LNP may be formulated in a capsule, film, or table having a coating. A capsule, film, or tablet including a composition described herein may have any useful size, tensile strength, hardness or density.

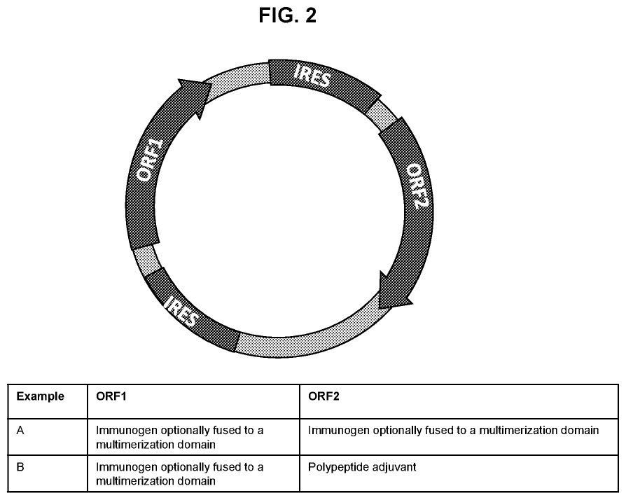

Additional exemplary lipids, formulations, methods, and characterization of LNPs are taught by WO2020/061457, WO2021/113777, and WO2021226597, each of which is incorporated herein by reference in its entirety. Further exemplary lipids, formulations, methods, and characterization of LNPs are taught by Hou et al. Lipid nanoparticles for mRNA delivery. Nat Rev Mater (2021). doi.org/10.1038/s41578-021-00358-0, which is incorporated herein by reference in its entirety (see, for example, exemplary lipids and lipid derivatives of FIG. 2 of Hou et al.).

In some embodiments, in vitro or ex vivo cell lipofections are performed using Lipofectamine MessengerMax (Thermo Fisher) or TranslT-mRNA Transfection Reagent (Mirus Bio). In certain embodiments, LNPs are formulated using the GenVoy_ILM ionizable lipid mix (Precision NanoSystems).

In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew, CHEM INT ED ENGL 51(34):8529-33 (2012), incorporated herein by reference in its entirety.

LNP formulations optimized for the delivery of CRISPR-Cas systems, e.g., Cas9-gRNA RNP, gRNA, Cas9 mRNA, are described in WO2019067992 and WO2019067910, both incorporated by reference, and are useful for delivery of circular polyribonucleotides and linear polyribonucleotides described herein.

Additional specific LNP formulations useful for delivery of nucleic acids (e.g., circular polyribonucleotides, linear polyribonucleotides) are described in U.S. Pat. Nos. 8,158,601 and 8,168,775, both incorporated by reference, which include formulations used in patisiran, sold under the name ONPATTRO.

Exemplary dosing of polyribonucleotide (e.g., a In embodiments, a polyribonucleotide (e.g., a circular polyribonucleotide, a linear polyribonucleotide) encoding at least a portion (e.g., an antigenic portion) of an immunogen or polypeptide described herein is formulated in an LNP, wherein: (a) the LNPs comprise a cationic lipid, a neutral lipid, a cholesterol, and a PEG lipid, (b) the LNPs have a mean particle size of between 80 nm and 160 nm, and (c) the polyribonucleotide comprises: (i) a 5′-cap structure; (ii) a 5′-UTR; (iii) N1-methyl-pseudouridine, cytosine, adenine, and guanine; (iv) a 3′-UTR; and (v) a poly-A region. In embodiments, the polyribonucleotide (e.g., circular polyribonucleotide, linear polyribonucleotide) formulated in an LNP is a vaccine.

Exemplary dosing of polyribonucleotide (e.g., a circular polyribonucleotide, a linear polyribonucleotide) may include about 0.1, 0.25, 0.3, 0.5, 1, 2, 3, 4, 5, 6, 8, 10, or 100 mg/kg (RNA). In some embodiments, a dose of a polyribonucleotide (e.g., a circular polyribonucleotide, a linear polyribonucleotide) immunogenic composition described herein is between 30-200 mcg, e.g., 30 mcg, 50 mcg, 75 mcg, 100 mcg, 150 mcg, or 200 mcg. Exemplary dosing of AAV including a polyribonucleotide (e.g., a circular polyribonucleotide, a linear polyribonucleotide) may include an MOI of about 10¹¹, 10¹², 10¹³, and 10¹⁴ vg/kg.

### Kits

In some aspects, the disclosure provides a kit. In some embodiments, the kit includes (a) a circular polyribonucleotide, an immunogenic composition, or a pharmaceutical composition described herein and optionally (b) informational material. In some embodiments, the kit further comprises an adjuvant described herein, which may be provided in a separate composition to be administered in combination with the circular polyribonucleotide, an immunogenic composition, or a pharmaceutical composition as part of a defined dosing regimen. The informational material may be descriptive, instructional, marketing or other material that relates to the methods described herein and/or the use of the pharmaceutical composition or circular polyribonucleotide for the methods described herein. The pharmaceutical composition or circular polyribonucleotide may comprise material for a single administration (e.g., single dosage form), or may comprise material for multiple administrations (e.g., a “multidose” kit).

The informational material of the kits is not limited in its form. In one embodiment, the informational material may include information about production of the pharmaceutical composition, the pharmaceutical drug substance, or the pharmaceutical drug product, molecular weight of the pharmaceutical composition, the pharmaceutical drug substance, or the pharmaceutical drug product, concentration, date of expiration, batch or production site information, and so forth. In one embodiment, the informational material relates to methods for administering a dosage form of the pharmaceutical composition. In one embodiment, the informational material relates to methods for administering a dosage form of the circular polyribonucleotide.

In addition to a dosage form of the pharmaceutical composition and circular polyribonucleotide described herein, the kit may include other ingredients, such as a solvent or buffer, a stabilizer, a preservative, a flavoring agent (e.g., a bitter antagonist or a sweetener), a fragrance, a dye or coloring agent, for example, to tint or color one or more components in the kit, or other cosmetic ingredient, and/or a second agent for treating a condition or disorder described herein. Alternatively, the other ingredients may be included in the kit, but in different compositions or containers than a pharmaceutical composition or circular polyribonucleotide described herein. In such embodiments, the kit may include instructions for admixing a pharmaceutical composition or nucleic acid molecule (e.g., a circular polyribonucleotide) described herein and the other ingredients, or for using a pharmaceutical composition or nucleic acid molecule (e.g., a circular polyribonucleotide) described herein together with the other ingredients.

In some embodiments, the components of the kit are stored under inert conditions (e.g., under Nitrogen or another inert gas such as Argon). In some embodiments, the components of the kit are stored under anhydrous conditions (e.g., with a desiccant). In some embodiments, the components are stored in a light blocking container such as an amber vial.

A dosage form of a pharmaceutical composition or nucleic acid molecule (e.g., a circular polyribonucleotide) described herein may be provided in any form, e.g., liquid, dried or lyophilized form. It is preferred that a pharmaceutical composition or nucleic acid molecule (e.g., a circular polyribonucleotide) described herein be substantially pure and/or sterile. When a pharmaceutical composition or nucleic acid molecule (e.g., a circular polyribonucleotide) described herein is provided in a liquid solution, the liquid solution preferably is an aqueous solution, with a sterile aqueous solution being preferred. When a pharmaceutical composition or nucleic acid molecule (e.g., a circular polyribonucleotide) described herein is provided as a dried form, reconstitution generally is by the addition of a suitable solvent. The solvent, e.g., sterile water or buffer, can optionally be provided in the kit.

The kit may include one or more containers for the composition containing a dosage form described herein. In some embodiments, the kit contains separate containers, dividers or compartments for the composition and informational material. For example, the pharmaceutical composition or circular polyribonucleotide may be contained in a bottle, vial, or syringe, and the informational material may be contained in a plastic sleeve or packet. In other embodiments, the separate elements of the kit are contained within a single, undivided container. For example, the dosage form of a pharmaceutical composition or nucleic acid molecule (e.g., a circular polyribonucleotide) described herein is contained in a bottle, vial or syringe that has attached thereto the informational material in the form of a label. In some embodiments, the kit includes a plurality (e.g., a pack) of individual containers, each containing one or more unit dosage forms of a pharmaceutical composition or circular polyribonucleotide described herein. For example, the kit includes a plurality of syringes, ampules, foil packets, or blister packs, each containing a single unit dose of a dosage form described herein.

The containers of the kits can be airtight, waterproof (e.g., impermeable to changes in moisture or evaporation), and/or light tight.

The kit optionally includes a device suitable for use of the dosage form, e.g., a syringe, pipette, forceps, measured spoon, swab (e.g., a cotton swab or wooden swab), or any such device.

The kits of the invention may include dosage forms of varying strengths to provide a subject with doses suitable for one or more of the initiation phase regimens, induction phase regimens, or maintenance phase regimens described herein. Alternatively, the kit may include a scored tablet to allow the user to administer divided doses, as needed.

### EXAMPLES

The following examples, which are intended to illustrate, rather than limit, the disclosure, are put forth to provide those of ordinary skill in the art with a description of how the compositions and methods described herein may be used, made, and evaluated. The examples are intended to be purely exemplary of the disclosure and are not intended to limit the scope of what the inventors regard as their invention.

### Example 1: Design, Generation, and Purification of Circular RNA Encoding Multimerization and Immunogen Sequences

This example describes the design and in vitro generation and purification of circular RNAs that encode a multimerization domain (e.g., a ferritin, bann, T4 foldon, or AaLS foldon domain) and an immunogen.

Circular RNAs were designed to include an internal ribosome entry site (IRES) and a nucleotide sequence encoding an immunogen fused to a multimerization domain. In this example, the DNA constructs were designed to include an IRES, a polynucleotide cargo, and a spacer element. The polynucleotide cargo included an ORE. The ORE included a secretion signal sequence or a native leader sequence, and a nucleotide sequence encoding an immunogen and a multimerization domain. Construct designs including a SARS-CoV-2 RBD immunogen, or an Influenza HA immunogen fused to a T4 foldon, bann, or ferritin multimerization domain are provided in Table 3.

TABLE 3
Construct designs

| ORF | ORF | | |
|---|---|---|---|
| Nucleic acid sequence | Amino acid sequence | Description | IRES |
| ATGGGAGTCAAAGTTCTGTTTGCCCTGA | MGVKVLFALICIAVAEAKRVQP | gLuc signal sequence; | EMCV |
| TCTGCATTGCTGTGGCCGAGGCCAAGAG | TESIVRFPNITNLCPFGEVFNA | SARS-COV-2 RBD | (SEQ ID NO: |
| AGTCCAACCAACAGAATCTATTGTTAGA | TRFASVYAWNRKRISNCVADYS | immunogen; | 79) or CVB3 |
| TTTCCTAATATTACAAACTTGTGCCCTT | VLYNSASFSTFKCYGVSPTKLN | T4 foldon | (SEQ ID NO: |
| TTGGTGAAGTTTTTAACGCCACCAGATT | DLCFTNVYADSFVIRGDEVRQI | multimerization domain | 80) |
| TGCATCCGTGTATGCTTGGAACAGGAAG | APGQTGKIADYNYKLPDDFTGC | | |
| AGAATCAGCAACTGTGTTGCTGATTATT | VIAWNSNNLDSKVGGNYNYLYR | | |
| CTGTCCTATATAATTCCGCATCATTTTC | LFRKSNLKPFERDISTEIYQAG | | |
| CACTTTTAAGTGTTATGGAGTGTCTCCT | STPCNGVEGFNCYFPLQSYGFQ | | |
| ACTAAATTAAATGATCTCTGCTTTACTA | PTNGVGYQPYRVVVLSFELLHA | | |
| ATGTCTATGCAGATTCATTTGTAATTAG | PATVCGPKKSTNLVKNKCVNFG | | |
| AGGTGATGAAGTCAGACAAATCGCTCCA | YIPEAPRDGQAYVRKDGEWVLL | | |
| GGGCAAACTGGAAAGATTGCTGATTATA | STFL (SEQ ID NO: 85) | | |
| ATTATAAATTACCAGATGATTTTACAGG | | | |
| CTGCGTTATAGCTTGGAATTCTAACAAT | | | |
| CTTGATTCTAAGGTTGGTGGTAATTATA | | | |
| ATTACCTGTATAGATTGTTTAGGAAGTC | | | |
| TAATCTCAAACCTTTTGAGAGAGATATT | | | |
| TCAACTGAAATCTATCAGGCCGGTAGCA | | | |
| CACCTTGTAATGGTGTTGAAGGTTTTAA | | | |
| TTGTTACTTTCCTTTACAATCATATGGT | | | |
| TTCCAACCCACTAATGGTGTTGGTTACC | | | |
| AACCATACAGAGTAGTAGTACTTTCTTT | | | |
| TGAACTTCTACATGCACCAGCAACTGTT | | | |
| TGTGGACCTAAAAAGTCTACTAATTTGG | | | |
| TTAAAAACAAATGTGTCAATTTCGGGTA | | | |
| TATCCCTGAAGCCCCCAGGGACGGCCAG | | | |
| GCTTACGTCAGAAAGGATGGAGAGTGGG | | | |
| TGCTCTTGAGCACCTTCCTG (SEQ ID | | | |
| NO: 84) | | | |
| atgggagtcaaagttctgtttgccctga | MGVKVLFALICIAVAEAKRVQP | gLuc signal sequence; | EMCV |
| tctgcattgctgtggccgaggccaagag | TESIVRFPNITNLCPFGEVFNA | SARS-COV-2 RBD | (SEQ ID NO: |
| agtccaaccaacagaatctattgttaga | TRFASVYAWNRKRISNCVADYS | immunogen; Bann | 79) |
| tttcctaatattacaaacttgtgccctt | VLYNSASFSTFKCYGVSPTKLN | multimerization domain | |
| ttggtgaagtttttaacgccaccagatt | DLCFTNVYADSFVIRGDEVRQI | | |
| tgcatccgtgtatgcttggaacaggaag | APGQTGKIADYNYKLPDDFTGC | | |
| agaatcagcaactgtgttgctgattatt | VIAWNSNNLDSKVGGNYNYLYR | | |
| ctgtcctatataattccgcatcattttc | LFRKSNLKPFERDISTEIYQAG | | |
| cacttttaagtgttatggagtgtctcct | STPCNGVEGFNCYFPLQSYGFQ | | |
| actaaattaaatgatctctgctttacta | PTNGVGYQPYRVVVLSFELLHA | | |
| atgtctatgcagattcatttgtaattag | PATVCGPKKSTNLVKNKCVNFI | | |
| aggtgatgaagtcagacaaatcgctcca | NHVGGTGGAIMAPVAVTRQLVG | | |
| gggcaaactggaaagattgctgattata | S (SEQ ID NO: 89) | | |
| attataaattaccagatgattttacagg | | | |
| ctgcgttatagcttggaattctaacaat | | | |
| cttgattctaaggttggtggtaattata | | | |
| attacctgtatagattgtttaggaagtc | | | |
| taatctcaaaccttttgagagagatatt | | | |
| tcaactgaaatctatcaggccggtagca | | | |
| caccttgtaatggtgttgaaggttttaa | | | |
| ttgttactttcctttacaatcatatggt | | | |
| ttccaacccactaatggtgttggttacc | | | |
| aaccatacagagtagtagtactttcttt | | | |
| tgaacttctacatgcaccagcaactgtt | | | |
| tgtggacctaaaaagtctactaatttgg | | | |
| ttaaaaacaaatgtgtcaatttcatcaa | | | |
| ccacgtgggcggaaccggggcgccatca | | | |
| tggcccccgtggccgtgacccggcagct | | | |
| ggtgggcagc (SEQ ID NO: 88) | | | |
| atgggagtcaaagttctgtttgccctga | MGVKVLFALICIAVAEAKRVQP | gLuc signal sequence; | EMCV |
| tctgcattgctgtggccgaggccaagag | TESIVRFPNITNLCPFGEVENA | SARS-Cov-2 RBD | (SEQ ID NO: |
| agtccaaccaacagaatctattgttaga | TRFASVYAWNRKRISNCVADYS | immunogen; Ferritin | 79) |
| tttcctaatattacaaacttgtgccctt | VLYNSASFSTFKCYGVSPTKLN | multimerization domain | |
| ttggtgaagtttttaacgccaccagatt | DLCFTNVYADSFVIRGDEVRQI | | |
| tgcatccgtgtatgcttggaacaggaag | APGQTGKIADYNYKLPDDFTGC | | |
| agaatcagcaactgtgttgctgattatt | VIAWNSNNLDSKVGGNYNYLYR | | |
| ctgtcctatataattccgcatcattttc | LFRKSNLKPFERDISTEIYQAG | | |
| cacttttaagtgttatggagtgtctcct | STPCNGVEGFNCYFPLQSYGFQ | | |
| actaaattaaatgatctctgctttacta | PTNGVGYQPYRVVVLSFELLHA | | |
| atgtctatgcagattcatttgtaattag | PATVCGPKKSTNLVKNKCVNFD | | |
| aggtgatgaagtcagacaaatcgctcca | IIKLLNEQVNKEMNSSNLYMSM | | |
| gggcaaactggaaagattgctgattata | SSWCYTHSLDGAGLFLFDHAAE | | |
| attataaattaccagatgattttacagg | EYEHAKKLIVFLNENNVPVQLT | | |
| ctgcgttatagcttggaattctaacaat | SISAPEHKFESLTQIFQKAYEH | | |
| cttgattctaaggttggtggtaattata | EQHISESINNIVDHAIKGKDHA | | |
| attacctgtatagattgtttaggaagtc | TFNFLQWYVSEQHEEEVLFKDI | | |
| taatctcaaaccttttgagagagatatt | LDKIELIGNENHGLYLADQYVK | | |
| tcaactgaaatctatcaggccggtagca | GIAKSRKS (SEQ ID NO: | | |
| caccttgtaatggtgttgaaggttttaa | 87) | | |
| ttgttactttcctttacaatcatatggt | | | |
| ttccaacccactaatggtgttggttacc | | | |
| aaccatacagagtagtagtactttcttt | | | |
| tgaacttctacatgcaccagcaactgtt | | | |
| tgtggacctaaaaagtctactaatttgg | | | |
| ttaaaaacaaatgtgtcaatttcgacat | | | |
| cattaagctgctcaacgagcaggtgaac | | | |
| aaggagatgaacagctccaacctgtaca | | | |
| tgagcatgagcagctggtgctacaccca | | | |
| cagcctggacggcgccggcctgttcctg | | | |
| tttgaccacgccgctgaggaatacgagc | | | |
| acgccaagaaactgatcgtgttcctgaa | | | |
| cgagaacaacgtgcccgtgcagctgacc | | | |
| agcatcagcgcccccgagcacaagttcg | | | |
| agagcctgacccagatcttccagaaggc | | | |
| ctacgagcacgagcagcacatcagcgag | | | |
| agcatcaacaacatcgtcgaccacgcca | | | |
| tcaagggcaaggaccacgccaccttcaa | | | |
| cttcctgcagtggtacgtgagcgagcag | | | |
| cacgaggaggaggtgctgttcaaggaca | | | |
| tcctggacaagatcgagctgatcggcaa | | | |
| cgagaaccacggcctgtacctggccgac | | | |
| cagtacgtgaagggcatcgccaagagcc | | | |
| gcaaaagt (SEQ ID NO: 86) | | | |
| ATGAAAGCCATTCTGGTCGTCCTCCTGT | MKAILVVLLYTFATANADTLCI | Native leader sequence; | EMCV |
| ATACCTTTGCCACAGCTAACGCTGATAC | GYHANNSTDTVDTVLEKNVTVT | Influenza A H1N1 | (SEQ ID NO: |
| CCTCTGCATTGGCTATCACGCTAACAAC | HSVNLLEDKHNGKLCKLRGVAP | hemagglutinin (HA) | 79) |
| AGTACCGATACCGTCGACACAGTGCTCG | LHLGKCNIAGWILGNPECESLS | immunogen; | |
| AGAAAAACGTCACCGTCACCCATAGCGT | TASSWSYIVETPSSDNGTCYPG | (A/California/07/2009); | |
| CAACCTCCTGGAAGACAAACATAACGGA | DFIDYEELREQLSSVSSFERFE | T4 foldon | |
| AAACTGTGTAAGCTCCGGGGAGTGGCTC | IFPKTSSWPNHDSNKGVTAACP | multimerization domain | |
| CCCTCCACCTCGGCAAGTGTAATATCGC | HAGAKSFYKNLIWLVKKGNSYP | | |
| CGGCTGGATTCTCGGCAATCCTGAGTGT | KLSKSYINDKGKEVLVLWGIHH | | |
| GAAAGCCTCAGCACAGCCAGTTCTTGGA | PSTSADQQSLYQNADAYVFVGS | | |
| GTTACATTGTCGAAACCCCTAGCAGCGA | SRYSKKFKPEIAIRPKVRDGEG | | |
| TAACGGAACCTGTTACCCTGGCGATTTC | RMNYYWTLVEPGDKITFEATGN | | |
| ATTGATTACGAGGAACTGCGCGAACAGC | LVVPRYAFAMERNAGSGIIISD | | |
| TCAGCTCTGTGAGCAGTTTCGAACGGTT | TPVHDCNTTCQTPKGAINTSLP | | |
| TGAGATTTTCCCTAAGACAAGCAGTTGG | FQNIHPITIGKCPKYVKSTKLR | | |
| CCTAACCACGACAGTAACAAAGGCGTCA | LATGLRNIPSIQSRGLFGAIAG | | |
| CCGCCGCTTGCCCTCACGCTGGAGCCAA | FIEGGWTGMVDGWYGYHHQNEQ | | |
| AAGCTTTTACAAAAACCTCATTTGGCTC | GSGYAADLKSTQNAIDEITNKV | | |
| GTCAAGAAGGGAAACAGTTACCCTAAGC | NSVIEKMNTQFTAVGKEFNHLE | | |
| TCAGCAAAAGCTATATTAACGATAAAGG | KRIENLNKKVDDGFLDIWTYNA | | |
| CAAAGAAGTCCTCGTCCTCTGGGGAATT | ELLVLLENERTLDYHDSNVKNL | | |
| CACCACCCTAGCACAAGCGCTGACCAAC | YEKVRSQLKNNAKEIGNGCFEF | | |
| AAAGTCTGTATCAAAACGCTGACGCTTA | YHKCDNTCMESVKNGTYDYPKY | | |
| CGTCTTTGTGGGAAGCTCTCGGTATAGC | SEEAKLNREEIDGVKLESTRIY | | |
| AAAAAGTTTAAGCCTGAGATTGCCATTC | GGGGSGYIPEAPRDGQAYVRKD | | |
| GGCCTAAGGTCCGCGACGGAGAGGGACG | GEWVLLSTFLGGGGSMKAILVV | | |
| CATGAATTATTATTGGACACTGGTCGAA | LLYTFATANADTLCIGYHANNS | | |
| CCCGGAGACAAAATCACATTCGAAGCCA | TDTVDTVLEKNVTVTHSVNLLE | | |
| CAGGCAATCTGGTCGTGCCTCGCTACGC | DKHNGKLCKLRGVAPLHLGKCN | | |
| TTTTGCTATGGAACGCAACGCTGGCAGT | IAGWILGNPECESLSTASSWSY | | |
| GGCATTATTATTAGCGATACCCCTGTCC | IVETPSSDNGTCYPGDFIDYEE | | |
| ACGATTGTAACACAACCTGTCAGACACC | LREQLSSVSSFERFEIFPKTSS | | |
| CAAAGGCGCTATCAATACCAGTCTGCCT | WPNHDSNKGVTAACPHAGAKSF | | |
| TTCCAAAACATTCACCCTATCACAATCG | YKNLIWLVKKGNSYPKLSKSYI | | |
| GAAAGTGTCCCAAATACGTCAAAAGTAC | NDKGKEVLVLWGIHHPSTSADQ | | |
| AAAACTGCGCCTGGCTACCGGACTGCGC | QSLYQNADAYVFVGSSRYSKKF | | |
| AACATTCCCAGTATCCAAAGCCGCGGCC | KPEIAIRPKVRDGEGRMNYYWT | | |
| TCTTCGGAGCCATTGCTGGATTCATTGA | LVEPGDKITFEATGNLVVPRYA | | |
| GGGAGGCTGGACCGGAATGGTCGACGGC | FAMERNAGSGIIISDTPVHDCN | | |
| TGGTACGGCTATCATCATCAAAACGAAC | TTCQTPKGAINTSLPFQNIHPI | | |
| AAGGAAGTGGATACGCCGCTGATCTCAA | TIGKCPKYVKSTKLRLATGLRN | | |
| GAGTACACAAAACGCTATTGACGAAATT | IPSIQSRGLFGAIAGFIEGGWT | | |
| ACCAATAAGGTCAACAGTGTGATTGAGA | GMVDGWYGYHHQNEQGSGYAAD | | |
| AAATGAATACCCAATTCACAGCCGTCGG | LKSTQNAIDEITNKVNSVIEKM | | |
| CAAAGAGTTTAACCATCTGGAAAAGCGC | NTQFTAVGKEFNHLEKRIENLN | | |
| ATCGAAAACCTCAACAAAAAGGTCGACG | KKVDDGFLDIWTYNAELLVLLE | | |
| ACGGATTCCTCGACATTTGGACATATAA | NERTLDYHDSNVKNLYEKVRSQ | | |
| CGCTGAACTGCTGGTCCTGCTCGAAAAC | LKNNAKEIGNGCFEFYHKCDNT | | |
| GAACGCACCCTCGATTATCACGATAGTA | CMESVKNGTYDYPKYSEEAKLN | | |
| ACGTCAAAAACCTCTACGAAAAGGTCCG | REEIDGVKLESTRIY (SEQ | | |
| GAGTCAGCTCAAGAATAACGCTAAGGAA | ID NO: 92) | | |
| ATCGGAAACGGCTGTTTTGAGTTTTACC | | | |
| ATAAGTGTGACAATACCTGTATGGAAAG | | | |
| CGTCAAAAACGGAACATACGATTACCCT | | | |
| AAGTATAGCGAAGAGGCTAAGCTCAACC | | | |
| GCGAGGAAATTGACGGAGTGAAACTGGA | | | |
| AAGCACACGGATTTACGGCGGCGGCGGC | | | |
| AGTGGCTATATCCCCGAGGCCCCCCGGG | | | |
| ACGGCCAGGCCTACGTGCGGAAGGACGG | | | |
| CGAGTGGGTGCTGTTGAGCACCTTCCTG | | | |
| GGGGGGGAGGCACATGAAGGCCATCCTG | | | |
| GTGGTTCTGCTGTACACCTTCGCCACCG | | | |
| CCAACGCCGACACCCTGTGCATCGGCTA | | | |
| CCACGCCAACAACAGCACCGACACCGTG | | | |
| GACACCGTGCTGGAGAAGAACGTGACCG | | | |
| TGACCCACAGCGTGAACCTGCTCGAGGA | | | |
| CAAGCACAACGGCAAGCTGTGCAAGCTG | | | |
| CGGGGCGTGGCCCCCCTGCACCTGGGCA | | | |
| AGTGCAATATTGCTGGCTGGATCCTGGG | | | |
| CAACCCCGAGTGCGAGAGCCTGAGCACC | | | |
| GCTAGCAGTTGGAGCTACATCGTGGAAA | | | |
| CACCCAGCTCTGACAACGGCACCTGCTA | | | |
| CCCCGGCGACTTCATCGACTACGAGGAG | | | |
| CTGCGGGAGCAGCTGAGCAGCGTGAGCT | | | |
| CCTTCGAGCGGTTCGAGATCTTCCCCAA | | | |
| GACCAGCAGCTGGCCCAATCACGATAGC | | | |
| AATAAGGGCGTGACCGCCGCCTGCCCCC | | | |
| ACGCCGGCGCCAAGAGCTTCTACAAGAA | | | |
| CCTGATCTGGCTGGTGAAGAAGGGCAAC | | | |
| AGCTACCCCAAGCTGAGCAAGAGCTACA | | | |
| TCAACGACAAGGGCAAGGAGGTCCTGGT | | | |
| CCTGTGGGGCATCCACCACCCCAGCACC | | | |
| AGCGCCGACCAGCAGAGCCTGTACCAGA | | | |
| ACGCCGACGCCTACGTGTTCGTGGGCAG | | | |
| CAGCCGGTACAGCAAGAAGTTCAAGCCC | | | |
| GAGATCGCCATCCGGCCCAAGGTGCGGG | | | |
| ACGGCGAGGGCCGGATGAACTACTACTG | | | |
| GACCCTGGTGGAGCCCGGCGACAAGATC | | | |
| ACCTTCGAGGCCACCGGCAACCTGGTGG | | | |
| TGCCCCGGTACGCCTTCGCCATGGAGCG | | | |
| GAACGCCGGCAGCGGCATCATAATCAGC | | | |
| GACACCCCCGTGCACGACTGCAACACCA | | | |
| CTTGCCAGACCCCCAAGGGCGCCATCAA | | | |
| CACCAGCCTGCCCTTCCAGAACATCCAC | | | |
| CCCATCACCATCGGCAAGTGCCCCAAGT | | | |
| ACGTGAAAAGCACCAAGCTGCGGCTGGC | | | |
| CACCGGCCTGCGGAACATCCCCAGCATC | | | |
| CAGAGCCGGGGCCTGTTCGGCGCTATTG | | | |
| CCGGCTTCATCGAGGGCGGGTGGACCGG | | | |
| CATGGTGGACGGCTGGTACGGCTACCAC | | | |
| CACCAGAACGAGCAGGGCAGCGGCTACG | | | |
| CCGCCGACCTCAAAAGTACCCAGAACGC | | | |
| CATCGACGAGATCACCAACAAGGTGAAC | | | |
| AGCGTGATCGAGAAGATGAACACCCAGT | | | |
| TCACCGCCGTGGGCAAGGAGTTCAACCA | | | |
| CCTGGAGAAGCGGATCGAGAACCTGAAC | | | |
| AAGAAAGTGGACGACGGCTTCCTGGACA | | | |
| TCTGGACCTACAACGCCGAGCTGCTGGT | | | |
| GCTGCTGGAGAACGAGCGGACCCTGGAC | | | |
| TACCACGACAGCAACGTGAAGAACCTGT | | | |
| ACGAGAAGGTGCGGAGCCAGCTGAAGAA | | | |
| CAACGCCAAGGAGATCGGCAACGGCTGC | | | |
| TTCGAGTTCTACCACAAGTGCGACAACA | | | |
| CCTGCATGGAGAGCGTGAAGAACGGCAC | | | |
| CTACGACTACCCCAAGTACAGCGAGGAG | | | |
| GCCAAGCTGAACCGGGAGGAAATCGACG | | | |
| GCGTGAAGCTGGAGAGCACCCGGATCTA | | | |
| C (SEQ ID NO: 93) | | | |
| atgaaggccatcctggtggtcctgctgt | MKAILVVLLYTFATANADTLCI | Native leader sequence; | EMCV |
| acaccttcgccaccgccaacgccgacac | GYHANNSTDTVDTVLEKNVTVT | Influenza A H1N1 | (SEQ ID NO: |
| cctgtgcatcggctaccacgccaacaat | HSVNLLEDKHNGKLCKLRGVAP | hemagglutinin (HA) | 79) |
| agcaccgacaccgtggacaccgtgctgg | LHLGKCNIAGWILGNPECESLS | immunogen | |
| agaagaacgtgaccgtgacccacagcgt | TASSWSYIVETPSSDNGTCYPG | (A/California/07/2009); | |
| gaacctgctggaggacaagcacaacggc | DFIDYEELREQLSSVSSFERFE | Bann multimerization | |
| aagctgtgcaagctgcggggcgtggccc | IFPKTSSWPNHDSNKGVTAACP | domain | |
| ccctgcacctgggcaagtgcaatattgc | HAGAKSFYKNLIWLVKKGNSYP | | |
| tggctggatcctgggcaaccccgagtgc | KLSKSYINDKGKEVLVLWGIHH | | |
| gagagcctgagcaccgccagctcctgga | PSTSADQQSLYQNADAYVFVGS | | |
| gctacatcgtggaaacacccagcagcga | SRYSKKFKPEIAIRPKVRDGEG | | |
| caacggcacctgctaccccggcgacttc | RMNYYWTLVEPGDKITFEATGN | | |
| atcgactacgaggaactgcgggagcagc | LVVPRYAFAMERNAGSGIIISD | | |
| tgagctctgtgagcagcttcgagcggtt | TPVHDCNTTCQTPKGAINTSLP | | |
| cgagatcttccccaagaccagcagctgg | FQNIHPITIGKCPKYVKSTKLR | | |
| cccaatcacgatagcaataagggcgtga | LATGLRNIPSIQSRGLFGAIAG | | |
| ccgccgcttgcccccacgccggcgccaa | FIEGGWTGMVDGWYGYHHQNEQ | | |
| gagcttctacaagaacctgatctggctg | GSGYAADLKSTQNAIDEITNKV | | |
| gtgaagaaaggcaacagctaccccaagc | NSVIEKMNTQFTAVGKEFNHLE | | |
| tgagcaagagctacatcaacgacaaggg | KRIENLNKKVDDGFLDIWTYNA | | |
| caaggaggtgctggtgctgtggggcatc | ELLVLLENERTLDYHDSNVKNL | | |
| caccatcccagcaccagcgccgaccagc | YEKVRSQLKNNAKEIGNGCFEF | | |
| aaagcctgtaccagaacgccgacgccta | YHKCDNTCMESVKNGTYDYPKY | | |
| cgtgttcgtgggcagctcccggtacagc | SEEAKLNREEIDGVKLESTRIY | | |
| aagaagttcaagcccgagatcgccatcc | GGGGSINHVGGTGGAIMAPVAV | | |
| ggcccaaggtgcgggacggcgagggccg | TRQLVG (SEQ ID NO: 90) | | |
| gatgaactactattggaccctggtggag | | | |
| cccggcgacaagatcaccttcgaggcca | | | |
| ccggcaacctggtggtgccccggtacgc | | | |
| cttcgccatggagcggaacgccggcagc | | | |
| ggcatcattatcagcgacacccccgtgc | | | |
| acgactgcaacaccacctgccagacccc | | | |
| caagggcgccatcaacaccagcctgccc | | | |
| ttccagaacatccaccccatcaccatcg | | | |
| gcaagtgccccaagtacgtgaaaagcac | | | |
| caagctgcggctggccaccggcctgcgg | | | |
| aacatccccagcatccagagccggggcc | | | |
| tgttcggcgctattgccggcttcatcga | | | |
| gggcggctggaccggcatggtggacggc | | | |
| tggtacggctaccaccaccagaacgagc | | | |
| agggcagcggctacgccgccgacctcaa | | | |
| aagtacccagaacgccatcgacgagatc | | | |
| accaacaaggtgaacagcgtgatcgaga | | | |
| agatgaacacccagttcaccgccgtggg | | | |
| caaggagttcaaccacctggagaagcgg | | | |
| atcgagaacctgaacaagaaggtggacg | | | |
| acggcttcctggacatctggacctacaa | | | |
| cgccgagctgctcgtgctgctggagaac | | | |
| gagcggaccctggactaccacgacagca | | | |
| acgtgaagaacctgtacgagaaggtgcg | | | |
| gagccagctgaagaacaacgccaaggag | | | |
| atcggcaacggctgcttcgagttctacc | | | |
| acaagtgcgacaacacctgcatggagag | | | |
| cgtgaagaacggcacctacgactacccc | | | |
| aagtacagcgaggaggccaagctgaacc | | | |
| gggaggagatcgacggcgtgaagctgga | | | |
| gagcacccggatctacggcggcgggggc | | | |
| agcatcaaccacgtgggcggcaccggcg | | | |
| gagccatcatggcccccgtggccgtgac | | | |
| ccggcagctggtgggc (SEQ ID NO: | | | |
| 91) | | | |
| ATGAAGGCCATCCTGGTGGTCCTGCTGT | MKAILVVLLYTFATANADTLCI | Native leader sequence; | EMCV |
| ACACCTTCGCCACCGCCAACGCCGACAC | GYHANNSTDTVDTVLEKNVTVT | Influenza A H1N1 | (SEQ ID NO: |
| CCTGTGCATCGGCTACCACGCCAACAAT | HSVNLLEDKHNGKLCKLRGVAP | hemagglutinin (HA) | 79) |
| AGCACCGACACCGTGGACACCGTGCTGG | LHLGKCNIAGWILGNPECESLS | immunogen; Ferritin | |
| AGAAGAACGTGACCGTGACCCACAGCGT | TASSWSYIVETPSSDNGTCYPG | multimerization domain | |
| GAACCTGCTGGAGGACAAGCACAACGGC | DFIDYEELREQLSSVSSFERFE | | |
| AAGCTGTGCAAGCTGCGGGGCGTGGCCC | IFPKTSSWPNHDSNKGVTAACP | | |
| CCCTGCACCTGGGCAAGTGCAATATTGC | HAGAKSFYKNLIWLVKKGNSYP | | |
| TGGCTGGATCCTGGGCAACCCCGAGTGC | KLSKSYINDKGKEVLVLWGIHH | | |
| GAGAGCCTGAGCACCGCCAGCTCCTGGA | PSTSADQQSLYQNADAYVFVGS | | |
| GCTACATCGTGGAAACACCCAGCAGCGA | SRYSKKFKPEIAIRPKVRDGEG | | |
| CAACGGCACCTGCTACCCCGGCGACTTC | RMNYYWTLVEPGDKITFEATGN | | |
| ATCGACTACGAGGAACTGCGGGAGCAGC | LVVPRYAFAMERNAGSGIIISD | | |
| TGAGCTCTGTGAGCAGCTTCGAGCGGTT | TPVHDCNTTCQTPKGAINTSLP | | |
| CGAGATCTTCCCCAAGACCAGCAGCTGG | FQNIHPITIGKCPKYVKSTKLR | | |
| CCCAATCACGATAGCAATAAGGGCGTGA | LATGLRNIPSIQSRGLFGAIAG | | |
| CCGCCGCTTGCCCCCACGCCGGCGCCAA | FIEGGWTGMVDGWYGYHHQNEQ | | |
| GAGCTTCTACAAGAACCTGATCTGGCTG | GSGYAADLKSTQNAIDEITNKV | | |
| GTGAAGAAAGGCAACAGCTACCCCAAGC | NSVIEKMNTQFTAVGKEFNHLE | | |
| TGAGCAAGAGCTACATCAACGACAAGGG | KRIENLNKKVDDGFLDIWTYNA | | |
| CAAGGAGGTGCTGGTGCTGTGGGGCATC | ELLVLLENERTLDYHDSNVKNL | | |
| CACCATCCCAGCACCAGCGCCGACCAGC | YEKVRSQLKNNAKEIGNGCFEF | | |
| AAAGCCTGTACCAGAACGCCGACGCCTA | YHKCDNTCMESVKNGTYDYPKY | | |
| CGTGTTCGTGGGCAGCTCCCGGTACAGC | SEEAKLNREEIDGVKLESTRIY | | |
| AAGAAGTTCAAGCCCGAGATCGCCATCC | GGGGSDIIKLLNEQVNKEMNSS | | |
| GGCCCAAGGTGCGGGACGGCGAGGGCCG | NLYMSMSSWCYTHSLDGAGLFL | | |
| GATGAACTACTATTGGACCCTGGTGGAG | FDHAAEEYEHAKKLIVFLNENN | | |
| CCCGGCGACAAGATCACCTTCGAGGCCA | VPVQLTSISAPEHKFESLTQIF | | |
| CCGGCAACCTGGTGGTGCCCCGGTACGC | QKAYEHEQHISESINNIVDHAI | | |
| CTTCGCCATGGAGCGGAACGCCGGCAGC | KGKDHATFNFLQWYVSEQHEEE | | |
| GGCATCATTATCAGCGACACCCCCGTGC | VLFKDILDKIELIGNENHGLYL | | |
| ACGACTGCAACACCACCTGCCAGACCCC | ADQYVKGIAKSRKS (SEQ ID | | |
| CAAGGGCGCCATCAACACCAGCCTGCCC | NO: 96) | | |
| TTCCAGAACATCCACCCCATCACCATCG | | | |
| GCAAGTGCCCCAAGTACGTGAAAAGCAC | | | |
| CAAGCTGCGGCTGGCCACCGGCCTGCGG | | | |
| AACATCCCCAGCATCCAGAGCCGGGGCC | | | |
| TGTTCGGCGCTATTGCCGGCTTCATCGA | | | |
| GGGCGGCTGGACCGGCATGGTGGACGGC | | | |
| TGGTACGGCTACCACCACCAGAACGAGC | | | |
| AGGGCAGCGGCTACGCCGCCGACCTCAA | | | |
| AAGTACCCAGAACGCCATCGACGAGATC | | | |
| ACCAACAAGGTGAACAGCGTGATCGAGA | | | |
| AGATGAACACCCAGTTCACCGCCGTGGG | | | |
| CAAGGAGTTCAACCACCTGGAGAAGCGG | | | |
| ATCGAGAACCTGAACAAGAAGGTGGACG | | | |
| ACGGCTTCCTGGACATCTGGACCTACAA | | | |
| CGCCGAGCTGCTCGTGCTGCTGGAGAAC | | | |
| GAGCGGACCCTGGACTACCACGACAGCA | | | |
| ACGTGAAGAACCTGTACGAGAAGGTGCG | | | |
| GAGCCAGCTGAAGAACAACGCCAAGGAG | | | |
| ATCGGCAACGGCTGCTTCGAGTTCTACC | | | |
| ACAAGTGCGACAACACCTGCATGGAGAG | | | |
| CGTGAAGAACGGCACCTACGACTACCCC | | | |
| AAGTACAGCGAGGAGGCCAAGCTGAACC | | | |
| GGGAGGAGATCGACGGCGTGAAGCTGGA | | | |
| GAGCACCCGGATCTACGGCGGCGGGGGC | | | |
| AGCGACATCATTAAGCTGCTCAACGAGC | | | |
| AGGTGAACAAGGAGATGAACAGCTCCAA | | | |
| CCTGTACATGAGCATGAGCAGCTGGTGC | | | |
| TACACCCACAGCCTGGACGGCGCCGGCC | | | |
| TGTTCCTGTTTGACCACGCCGCTGAGGA | | | |
| ATACGAGCACGCCAAGAAACTGATCGTG | | | |
| TTCCTGAACGAGAACAACGTGCCCGTGC | | | |
| AGCTGACCAGCATCAGCGCCCCCGAGCA | | | |
| CAAGTTCGAGAGCCTGACCCAGATCTTC | | | |
| CAGAAGGCCTACGAGCACGAGCAGCACA | | | |
| TCAGCGAGAGCATCAACAACATCGTCGA | | | |
| CCACGCCATCAAGGGCAAGGACCACGCC | | | |
| ACCTTCAACTTCCTGCAGTGGTACGTGA | | | |
| GCGAGCAGCACGAGGAGGAGGTGCTGTT | | | |
| CAAGGACATCCTGGACAAGATCGAGCTG | | | |
| ATCGGCAACGAGAACCACGGCCTGTACC | | | |
| TGGCCGACCAGTACGTGAAGGGCATCGC | | | |
| CAAGAGCCGCAAAAGT (SEQ ID NO: | | | |
| 95) | | | |

DNA constructs were also designed to include a combination of a modified CVB3 IRES (SEQ ID NO: 80) and an RSV F immunogen (with its native leader sequence) or a human MPV F immunogen (with its native leader sequence) fused to a T4 foldon multimerization domain as the ORE.

In this example, the circular RNAs were generated by self-splicing using a method described herein. Unmodified linear RNA was synthesized by in vitro transcription using T7 RNA polymerase from a DNA template including the motifs listed above in the presence of 7.5 mM of NTP. Synthesized linear RNA was purified with an RNA clean up kit (New England Biolabs, T2050). Self-splicing occurred during transcription; no additional reaction was required. The circular RNAs were purified by urea polyacrylamide gel electrophoresis (Urea-PAGE) or by reversed phase column chromatography.

### Example 2: In Vitro Expression of Immunogen with Multimerization Domain

This example demonstrates expression of an immunogen with a multimerization domain from circular RNAs in mammalian cells.

Circular RNA encoding a SARS-CoV-2 RBD immunogen fused to a T4 foldon multimerization domain (Nucleic acid SEQ ID NO: 84; Amino acid SEQ ID NO: 85) domain (circRNA-RBD-Foldon) was produced as described in Example 1. Circular RNA encoding the SARS-CoV-2 RBD immunogen without a multimerization domain (Nucleic acid SEQ ID NO: 82; Amino acid SEQ ID NO: 83) (circRNA-RBD (monomer)) was produced and purified by the methods described herein. Both constructs included EMCV having a nucleic acid sequence of

(SEQ ID NO: 79)
ACGTTACTGGCCGAAGCCGCTTGGAATAAGGCCGGTGTGCGTTTGTCTAT
ATGTTATTTTCCACCATATTGCCGTCTTTTGGCAATGTGAGGGCCCGGAA
ACCTGGCCCTGTCTTCTTGACGAGCATTCCTAGGGGTCTTTCCCCTCTCG
CCAAAGGAATGCAAGGTCTGTTGAATGTCGTGAAGGAAGCAGTTCCTCTG
GAAGCTTCTTGAAGACAAACAACGTCTGTAGCGACCCTTTGCAGGCAGCG
GAACCCCCCACCTGGCGACAGGTGCCTCTGCGGCCAAAAGCCACGTGTAT
AAGATACACCTGCAAAGGCGGCACAACCCCAGTGCCACGTTGTGAGTTGG
ATAGTTGTGGAAAGAGTCAAATGGCTCTCCTCAAGCGTATTCAACAAGGG
GCTGAAGGATGCCCAGAAGGTACCCCATTGTATGGGATCTGATCTGGGGC
CTCGGTGCACATGCTTTACATGTGTTTAGTCGAGGTTAAAAAACGTCTAG
GCCCCCCGAACCACGGGGACGTGGTTTTCCTTTGAAAAACACGATGATAA
TA

as the IRES element.

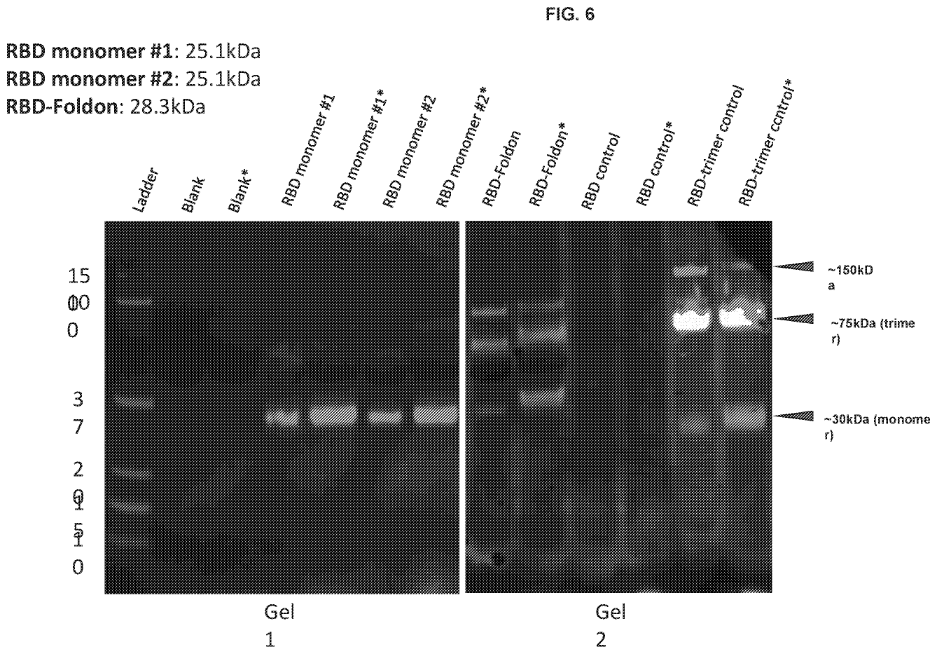

Circular RNAs were transfected into HEK293T using Lipofectamine MessengerMax (Invitrogen, LMRNA015) according to manufacturer's instructions. MessengerMax alone (Blank) was used as a control. Recombinant SARS-CoV-2 RBD protein (Sino Biological; 40592-V08H) and SARS-CoV-2 RBD trimer protein (Acro Biosystems; SPD-C52M5-200 ug) were also used as controls (RBD control and RBD-trimer control, respectively). Cell supernatants were harvested after 24 hours. Samples were run on a gel via SDS-PAGE under non-denaturing or denaturing conditions (loading buffer, without versus with 3-mercaptoethanol). Western blotting was then performed, with 2G1 anti-RBD monoclonal antibody (Abcam, ab277624) as the primary antibody and fluorescent ‘680CW’ goat anti-mouse IgG (Licor, 926-68070) as the secondary antibody. Results are shown in FIG. 6, with an asterisk identifying the samples that were run under denaturing conditions (i.e., the samples including beta-mercaptoethanol in the loading buffer).

FIG. 6 shows that circRNA-RBD expresses RBD monomer in HEK293T and circRNA encoding a SARS-CoV-2 RBD immunogen fused to a T4 Foldon multimerization domain expresses and forms trimeric structures in vitro (trimers).

### Example 3: In Vivo Expression of Immunogens from Circular RNA in Mouse Model

This example demonstrates in vivo expression of immunogens with and without a multimerization domain from circular RNAs.

Circular RNA encoding a SARS-CoV-2 RBD immunogen fused to a Foldon multimerization domain (Nucleic acid SEQ ID NO: 84; Amino acid SEQ ID NO: 85) domain (circRNA-RBD-Foldon (trimer)) was produced as described in Example 1. Circular RNA encoding the SARS-CoV-2 RBD immunogen without a multimerization domain (Nucleic acid SEQ ID NO: 82; Amino acid SEQ ID NO: 83) (circRNA-RBD (monomer)) was produced and purified by the methods described herein. Both constructs included EMCV (SEQ ID NO: 79) as the IRES element.

Purified circular RNAs were formulated into a lipid nanoparticle to obtain circular RNA preparations. Briefly, circular RNA was diluted in 25 mM acetate buffer pH=4 (filtered through 0.2 um filter) to a concentration of 0.2 μg/uL. Lipid nanoparticles (LNPs) were formulated by first dissolving the ionizable lipid (e.g., ALC0315), cholesterol, DSPC, and DMG-PEG2000 in ethanol (filtered through 0.2 um sterile filter) in a molar ratio of 50/38.5/10/1.5 mol %. The final ionizable lipid/RNA weight ratio was 6/1 w/w. The lipid and RNA solutions were mixed in a micromixer chip using microfluidics system with a flow rate ratio of 3/1 buffer/ethanol and a total flow rate of 1 ml/min. The LNPs were then dialyzed in PBS pH=7.4 for 3 hours to remove ethanol. The LNPs were concentrated to the desired RNA concentration using Amicon centrifugation filters, 100 kDa cut off, as necessary.

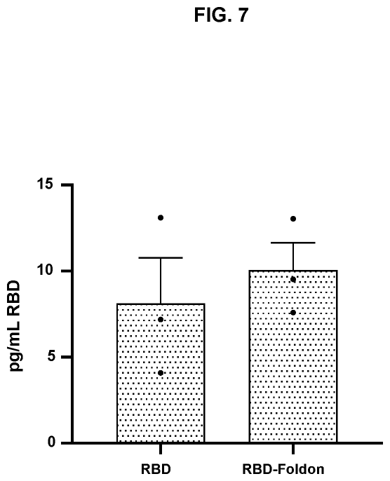

Three Balb/C mice per group (n=3) were administered a 5-μg dose of a circular RNA preparation via intramuscular injection at day 0 (prime) and day 28 (boost). At 24 hours post-prime, serum samples were collected from each mouse. Expression of monomer and trimer was measured using a SARS-CoV-2 RBD immunogen-specific ELISA. ELISA plates were coated overnight with capture antibody (SinoBiological, 40150-D003). Plates were blocked with TBST+2% BSA and serum diluted in blocking buffer then added to plate. RBD was detected with HRP-conjugated detection antibody (40150-D001-H, Sinobiological). Data are shown in FIG. 7 as the mean of three animals per group. Similar levels of SARS-CoV-2 RBD and a SARS-CoV-2 RBD immunogen fused to a T4 Foldon multimerization domain were detected in the serum 24 hours post-prime.

The results show that SARS-CoV-2 RBD and a SARS-CoV-2 RBD immunogen fused to a T4 Foldon multimerization domain were expressed at comparable levels from circular RNAs in a mouse model.

### Example 4: Immunogenicity of Immunogens from Circular RNA in Mouse Model

This example demonstrates circular RNA encoding an immunogen with a multimerization domain induces an immunogen-specific response in mice.

Serum samples were isolated from mice administered the circular RNA preparations as described in Example 3, at days 14, 27, 35, and 42. Binding antibody responses were measured by ELISA as follows: Individual serum samples were assayed for the presence of RBD-specific IgG. ELISA plates were coated overnight at 4° C. with SARS-CoV-2 RBD (Sino Biological, 40592-V08B; 100 ng) in 100 μL of 1× coating buffer (Biolegend, 421701) or SARS-CoV-2-RBD (Sinobiological, 40592-V08H). The plates were then blocked for 1 hour with blocking buffer (TBS with 2% BSA and 0.05% Tween 20). Serum samples were serially diluted 8 times (4-fold dilutions from 500 to 8,192,000) then added to each well in 100 μL blocking buffer and incubated at room temperature for 1 hour. After washing three times with 1× Tris-buffered saline with Tween® detergent (TBS-T), plates were incubated with anti-mouse IgG HRP detection antibody (Abcam, ab97023) for 1 hour followed by three washes with TBS-T, then addition of tetramethylbenzene (Biolegend, 421101). The ELISA plate was allowed to react for 10-20 minutes and then quenched using 0.2N sulfuric acid. The optical density (O.D.) value was determined at 450 nm. The endpoint titer was defined as the last dilution with an absorbance value of 4-fold over background.

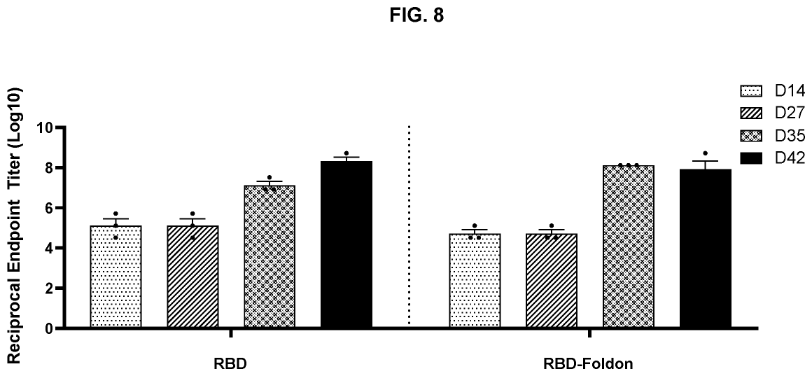

FIG. 8 shows the mean endpoint titer at days 14, 27, 35 and 42 post-immunization with circRNA encoding a SARS-CoV-2RBD or circRNA encoding a SARS-CoV-2 RBD immunogen fused to a T4 Foldon multimerization domain. At day 42 post-injection, the mice were sacrificed, and spleens were harvested and tested for SARS-CoV-2 RBD T cells responses by ELISpot assay following manufacturer's protocol (Mabtech, 3321-4HPT-10). Briefly, the spleens were harvested and processed into a single cell suspension. Splenocytes were plated at 0.5 M cells per well on IFN-γ ELISpot plates. Splenocytes were either unstimulated or stimulated with RBD 1 μg/mL of or RBD peptide pools (JPT, PM-WCPV-S-RBD-2). Cells were cultured overnight, and the plates were developed the next day according to manufacturer's protocol.

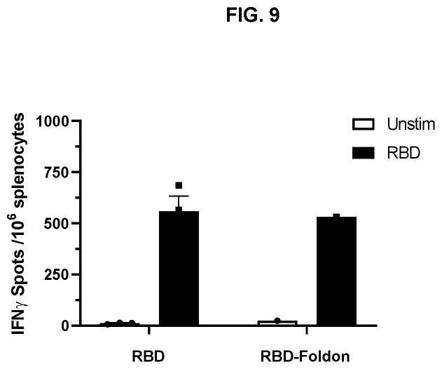

The results show that both circRNA a SARS-CoV-2 RBD and circRNA encoding a SARS-CoV-2 RBD immunogen fused to a T4 Foldon multimerization domain prime T cell responses in mice after immunization (FIG. 9).

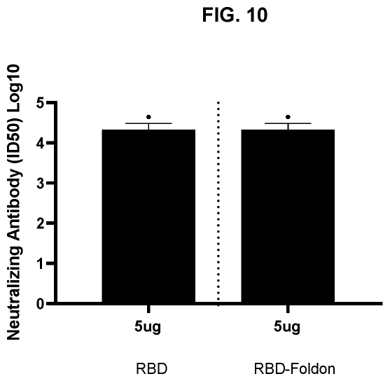

The neutralizing antibody titer from serum collected on Day 42 post-injection was tested in a Plaque Reduction Neutralization Test (PRNT). Briefly serum was serially diluted, mixed with SARS-CoV-2 viral stock and placed on Vero E6 cells. Plates were overlayed with low-melting point agarose and incubated for 3 days, followed by fixation and staining with crystal violet. The neutralization titer was reported as ID50: the dilution at which the serum reduces the number of plaques by fifty percent (50%). FIG. 10 shows that both circRNA a SARS-CoV-2 RBD and circRNA encoding a SARS-CoV-2 RBD immunogen fused to a T4 Foldon multimerization domain generated neutralizing antibody against SARS-CoV-2.

The results of this Example 4 demonstrate that immunogens with a multimerization domain expressed from circular RNA induces an immunogen-specific response in mice.

### Example 5: In Vivo Expression of Immunogens from Circular RNA in Non-Human Primate Model

This example demonstrates in vivo expression of immunogens with a multimerization domain from circular RNA in a non-human primate (NHP).

Circular RNA encoding a SARS-CoV-2 RBD immunogen fused to a T4 Foldon multimerization domain (Nucleic acid SEQ ID NO: 84; Amino acid SEQ ID NO: 85) and including EMCV (SEQ ID NO: 79) as the IRES, was produced as described in Example 1.

Circular RNAs were formulated in LNP as described in Example 3 (LNP-formulated circular RNAs). Circular RNAs were also formulated by admixing with an equal volume of AddaSO3™ adjuvant solution (adjuvanted circular RNAs).

Three cynomolgus monkeys (n=3) per group were administered either a 30 μg or 100 μg dose of LNP-formulated circular RNA, or a 1000 μg dose of adjuvanted circular RNA via intramuscular injection at day 0 (prime) and day 28 (boost). At 6 hours, Day 1, Day 4, Day 6 post-prime, serum samples were collected from each monkey. SARS-CoV-2 RBD immunogen fused to a T4 Foldon multimerization domain levels were measured using a SARS-CoV-2 Spike immunoassay according to manufacturer's protocol (MDS, S-PLEX SARS-CoV-2 Spike Kit, K150ADJS-2).

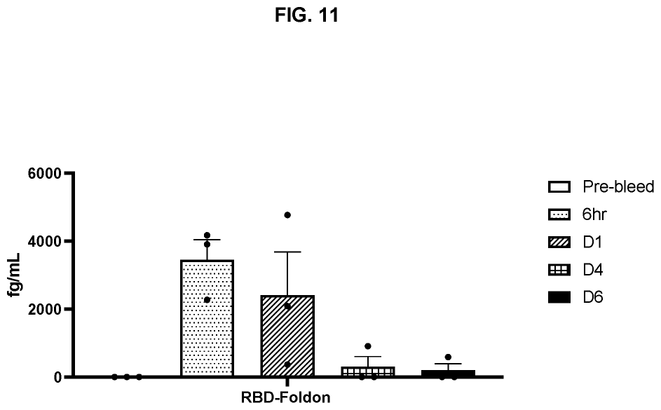

SARS-CoV-2 RBD immunogen fused to a T4 Foldon multimerization domain expression was not detected in serum of monkeys that were administered adjuvanted circular RNAs. SARS-CoV-2 RBD immunogen fused to a T4 Foldon multimerization domain was detected in serum of monkeys that received 100 μg of LNP-formulated circular RNA (FIG. 11, data shown as the mean of three animals per group). SARS-CoV-2 RBD immunogen fused to a T4 Foldon multimerization domain levels of ˜3500 fg/mL were detected at 6 hours post-prime, with SARS-CoV-2 RBD immunogen fused to a T4 Foldon multimerization domain concentrations decreasing over the course of the 6 days post-prime during which samples were collected.

### Example 6: Immunogenicity of Immunogens from Circular RNA in Non-Human Primate Model

This example demonstrates circular RNA encoding an immunogen with a multimerization domain induces an immunogen-specific response in a non-human primate (NHP).

Serum samples were isolated from monkeys administered 100 μg dose of LNP-formulated circular RNA or 1000 μg dose of adjuvanted circular RNA as described in Example 5, at Days 14, 35, 42, and 56 post-prime.

Binding antibody was detected using a SARS-CoV-2 Spike immunoassay according to manufacturer's protocol (MDS, S-PLEX SARS-CoV-2 Spike Kit, K150ADJS-2). NHP serum was diluted at 1:1000 or 1:5000 or 1:50 000. Binding antibody concentration was interpolated using the pooled serum standard and results were reported as Geometric Mean International Units per mL.

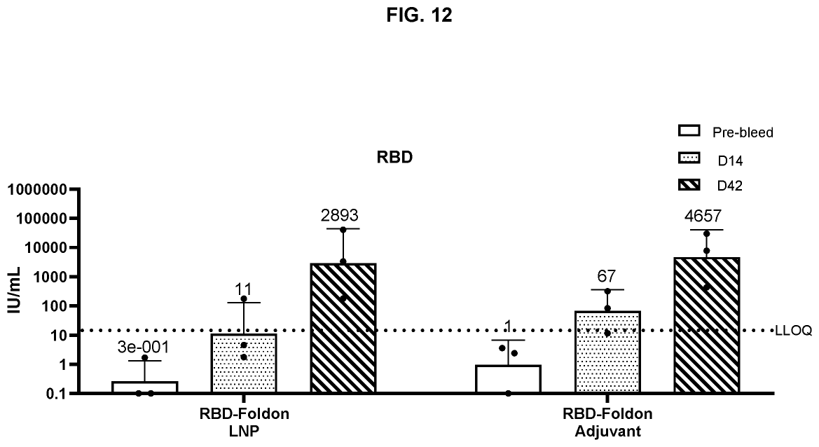

FIG. 12 shows the geometric mean of antibody at pre-bleed, at Day 14 and Day 42 post-immunization with adjuvanted circular RNA and LNP-formulated circular RNA. The results show that LNP-formulated circular RNA primed RBD-specific binding antibodies at Day 42 post-prime, and that adjuvanted circular RNA primed similar levels of RBD-specific binding antibodies than obtained.

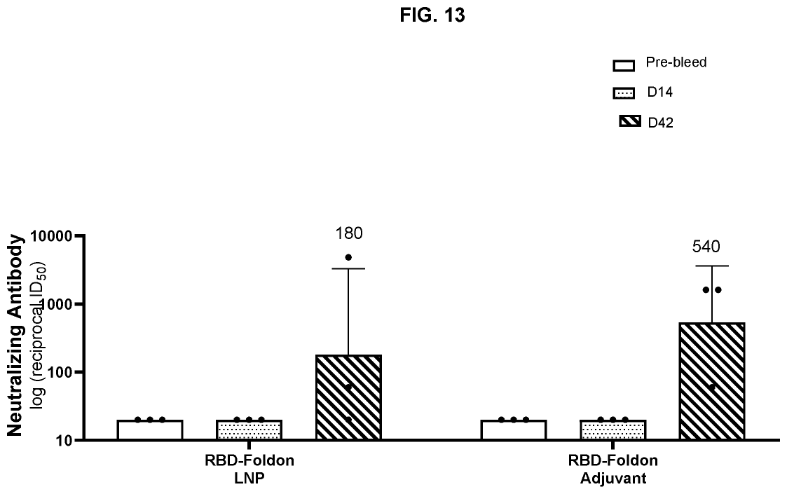

The neutralizing antibody titer from serum collected on pre-bleed, Day 14 and Day 42 post-prime was tested in a Plaque Reduction Neutralization Test (PRNT). Briefly serum was serially diluted, mixed with SARS-CoV-2 viral stock and placed on Vero E6 cells. Plates were overlayed with low-melting point agarose and incubated for 3 days, followed by fixation and staining with crystal violet. The neutralization titer was reported as ID50: the dilution at which the serum reduces the number of plaques by fifty percent (50%). Data are shown in FIG. 13 as geometric mean neutralizing titer at pre-bleed, and Day 14 and Day 42 post-boost.

FIG. 13 shows that both LNP-formulated circular RNA encoding a SARS-CoV-2 RBD immunogen fused to a T4 Foldon multimerization domain and adjuvanted circular RNA encoding SARS-CoV-2 RBD immunogen fused to a T4 Foldon multimerization domain primed neutralizing SARS-CoC-2 neutralizing antibodies.

### Example 7: T Cell Responses of Immunogens from Circular RNA in Non-Human Primate Model

Peripheral blood mononuclear cells (PBMCs) are harvested and frozen pre-immunization and at D42 post-immunization. PBMCs were thawed and an ELISpot assay used to detect the presence of SARS-CoV-2 RBD-specific T cells. 0.2 M cells are plated per well on IFN-γ or IL-4 ELISpot plates (ImmunoSpot) and are either left unstimulated or stimulated with SARS-CoV-2 peptide pools (JPT, PM-WCPVS-2). ELISPOT plates are processed according to manufacturer's protocol.

### Example 8: Design, Generation, and Purification of Circular RNA Encoding Multimerization and Immunogen Sequences

This example describes the design and in vitro generation and purification of circular RNAs that encode a multimerization domain (e.g., ferritin, bann, T4 foldon, AaLS) and an immunogen (e.g., a gE VZV immunogen or a SARS-CoV2 RBD immunogen). Circular RNAs are designed to include an IRES, and a nucleic acid sequence encoding a VZV or another immunogen fused to a multimerization domain. Some of the circular RNAs encode a native leader sequence or a secretion signal.

In this example, circular RNAs are generated by one of two exemplary methods and purified again with the RNA purification system.

### Exemplary Method 1: DNA-Splint Ligation

This method produces a circular RNA by splint-ligation. RppH-treated linear RNA is circularized using a splint DNA. Unmodified linear RNA is synthesized by in vitro transcription using T7 RNA polymerase from a DNA segment. Transcribed RNA is purified with an RNA purification system (New England Biolabs), treated with RNA 5′ phosphohydrolase (RppH) (New England Biolabs, M0356) following the manufacturer's instructions. Alternately or in addition, the RNA is transcribed in an excess of GMP over GTP.

Splint-ligation is performed as follows: circular RNA is generated by treatment of the transcribed linear RNA and a DNA splint between 10 and 40 nucleotides in length using an RNA ligase. To purify the circular RNAs, ligation mixtures are resolved on 4% denaturing PAGE and RNA bands corresponding to each circular RNA are excised. Excised RNA gel fragments are crushed, and RNA eluted with gel elution buffer (0.5 M Sodium Acetate, 0.1% SDS, 1 mM EDTA) for one hour at 37° C. Alternately or in addition, the circular RNA is purified by column chromatography. Supernatant is harvested, and RNA is eluted again by adding gel elution buffer to the crushed gel and incubated for one hour. Gel debris is removed by centrifuge filters and is precipitated with ethanol. Agarose gel electrophoresis is used as a quality control measurement for validating purity and circularization.

### Exemplary Method 2: Circularization by Self-Splicing Intron

This method produces a circular RNA by self-splicing. The circular RNA is generated in vitro. Unmodified linear RNA is in vitro transcribed from a DNA template including all the motifs listed above. In vitro transcription reactions included 1 μg of template DNA T7 RNA polymerase promoter, 10× T7 reaction buffer, 7.5 mM ATP, 7.5 mM CTP, 7.5 mM GTP, 7.5 mM UTP, 10 mM DTT, 40 U RNase Inhibitor, and T7 enzyme. Transcription is carried out at 37° C. for 4 h. Transcribed RNA is DNase treated with 1 U of DNase I at 37° C. for 15 min. To favor circularization by self-splicing, additional GTP is added to a final concentration of 2 mM, incubated at 55° C. for 15 min. RNA is then column purified and visualized by UREA-PAGE.

### Example 9: In Vitro Expression of Immunogen with Multimerization Domain

This example describes expression of immunogens from circular RNAs in mammalian cells. To measure expression of immunogens from the RNA constructs with multimerization domains, immunogen-encoding circular RNA is produced and purified according to the methods described herein. Circular RNA (1 picomole) is transfected into HEK293T (200,000 cells per well in a 24 well plate in serum-free media) using MessengerMax (Invitrogen, LMRNA). Cell supernatant is harvested after 24 hours. The ELISA is performed as follows: a capture antibody is coated onto ELISA plates (MaxiSorp 442404, 96-well) overnight at 4° C. in 100 μL PBS. After washing three times with TBS-T, the plates are blocked for 1 hour with blocking buffer (TBS with 2% FBS and 0.05% Tween 20). Supernatant dilutions are then added to each well in 100 μL blocking buffer and incubated at room temperature for 1 hour. After washing three times with TBS-T, plates are incubated with HRP detection antibody for 1 hour at room temperature. Tetramethylbenzene (Pierce 34021) is added to each well, allowed to react for 5-15 minutes and then quenched with 2N sulfuric acid. The optical density (OD) value will be determined at 450 nm. Validation of successful immunogen multimerization is determined by running non-denaturing Blue Native PAGE on supernatants from cells transfected with circular RNAs encoding immunogen with or without a multimerization domain. Blue Native gel is transferred to polyvinylidene fluoride (PVDF) membrane for Western blotting, probing for immunogen with a specific primary antibody followed by an anti-isotype fluorescently tagged secondary antibody. Multimerized immunogen will be expected to appear at a higher molecular weight than non-multimerized immunogen.

### Example 10: In Vivo Expression of Immunogens from Circular RNA in Mouse Model

This example demonstrates in vivo expression of immunogens with and without a multimerization domain from circular RNAs. The circular RNAs are designed and produced as described in Example 8. The circular RNAs are formulated into lipid nanoparticles to obtain circular RNA preparations. Different concentrations of circular RNA preparation are administered to 3 mice per group including a group with circular RNA not encoding a multimerization domain. Circular RNA preparations are administered to mice intramuscularly at day 0 with a second administration 4 weeks later. A control group of mice are treated with vehicle and no circular RNA. Blood samples are taken throughout the time course to monitor immunogen-specific antibody titers in serum by ELISA. Blood (˜100 μl) is collected by submandibular bleed from each mouse into dry tubes, at 1 day, 2 days, 3 days, and 7 days, and then weekly for 9 weeks post-dosing. Serum is collected by centrifugation for 25 minutes at 1,300 g at 4° C. and immunogen levels are measured by ELISA following manufacturer's instructions.

At the terminal time point the mice are sacrificed. Spleens and blood are harvested and splenocytes and serum are tested for immunogen specificity by flow cytometry and ELISpot. The collected serum is tested in an infection inhibition assay to determine neutralizing capacity of serum antibodies.

### Example 11: In Vivo Expression of Immunogens from Circular RNA Delivered with Adjuvant in Mouse Model

This example demonstrates in vivo expression of immunogens with and without multimerization domains from circular RNAs with immune enhancement by deliver alongside an adjuvant, such as AddaSO3™ adjuvant. The circular RNAs are designed and produced as described in Example 8. The circular RNAs are formulated by admixing with an equal volume of AddaSO3™ adjuvant solution. The circular RNA/adjuvant preparations are administered to mice intramuscularly at day 0 with a second administration 4 weeks later. A control group of mice are treated with vehicle and no circular RNA. Additional control groups may be included containing circular RNA but no adjuvant or circular RNA formulated in LNPs. Blood samples are taken throughout the time course to monitor immunogen-specific antibody titers in serum by ELISA. Blood (˜100 μl) is collected by submandibular bleed from each mouse into dry tubes, at 1 day, 2 days, 3 days, and 7 days, and then weekly for 9 weeks post-dosing. Serum is collected by centrifugation for 25 minutes at 1,300 g at 4° C. and immunogen levels are measured by ELISA following manufacturer's instructions.

At the terminal time point the mice are sacrificed. Spleens—are harvested and splenocytes are tested for immunogen-specific T cells by flow cytometry and ELISpot. The collected serum is tested in an infection inhibition assay to determine neutralizing capacity of serum antibodies.

### Example 12: Evaluation of Different Doses of SARS-Cov-2 Receptor Binding Domain (RBD) Circular RNA in Mice with and without Foldon Domains

This example measures immune response in mice intramuscularly or intradermally injected with different formulations and different doses of RBD-encoding circular RNA (circRNA) with and without foldon sequences. Mice are split into thirteen equal groups: Control injected with PBS and no circular RNA, circRNA-RBD 0.1 μg LNP formulation, circRNA-RBD 1.0 μg LNP formulation, circRNA-RBD 0.1 μg with foldon LNP formulation, circRNA-RBD 1.0 μg with foldon LNP formulation, circRNA-RBD 0.1 μg AddaSO3 formulation, circRNA-RBD 1.0 μg AddaSO3 formulation, circRNA-RBD 0.1 μg with foldon AddaSO3 formulation, circRNA-RBD 1.0 μg with foldon AddaSO3 formulation, circRNA-RBD 0.1 μg CpG+Alum formulation, circRNA-RBD 1.0 μg CpG+Alum formulation, circRNA-RBD 0.1 μg with foldon CpG+Alum formulation, and circRNA-RBD 1.0 μg with foldon CpG+Alum formulation. The formulations are evaluated for their ability to demonstrate an antibody response resulting from administration of circular RNA encoding the expression of the multiple antigens.

Blood (˜100 μl) is collected by submandibular bleed from each mouse into dry tubes at 4 hours post injection. Two mice are terminated from each group 24 hours post injection, and the remaining mice are bled for samples on day 14, 28, and 35. Mice are sacrificed on day 49 with a final bleed and spleen collection. Spleens are harvested and tested for RBD immunogen-specific T cells by flow cytometry and ELISpot.

### OTHER EMBODIMENTS

Various modifications and variations of the described compositions, methods, and uses of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

All publications, patents, and patent applications are herein incorporated by reference in their entirety to the same extent as if each individual publication, patent, or patent application was specifically and individually indicated to be incorporated by reference in its entirety.

1. A circular polyribonucleotide comprising an open reading frame comprising a sequence encoding an immunogen and a sequence encoding a multimerization domain.

2. The circular polyribonucleotide of claim 1, wherein the open reading frame comprises, arranged in the following 5′-to-3′ order:
(a) a first sequence encoding an immunogen and second sequence encoding a multimerization domain;
(b) a first sequence encoding an immunogen, a second sequence encoding a multimerization domain, and a third sequence encoding an immunogen;
(c) a first sequence encoding an immunogen, a second sequence encoding a multimerization domain, a third sequence encoding an immunogen, and a fourth sequence encoding a multimerization domain;
(d) a first sequence encoding an immunogen, a second sequence encoding a multimerization domain, and a third sequence encoding a multimerization domain;
(e) a first sequence encoding a multimerization domain and second sequence encoding an immunogen;
(f) a first sequence encoding a multimerization domain, a second sequence encoding an immunogen, and a third sequence encoding a multimerization domain;
(g) a first sequence encoding a multimerization domain, a second sequence encoding an immunogen, a third sequence encoding a multimerization domain, and a fourth sequence encoding an immunogen; or
(h) a first sequence encoding a multimerization domain, a second sequence encoding a multimerization domain, and a third sequence encoding an immunogen.

3. The circular polyribonucleotide of claim 1 or 2, wherein the multimerization domain or each multimerization domain comprises a T4 foldon domain.

4. The circular polyribonucleotide of claim 1 or 2, wherein the multimerization domain or each multimerization domain comprises a ferritin domain.

5. The circular polyribonucleotide of claim 1 or 2, wherein the multimerization domain or each multimerization domain comprises a β-annulus peptide.

6. The circular polyribonucleotide of claim 1 or 2, wherein the multimerization domain or each multimerization domain comprises an AaLS peptide.

7. The circular polyribonucleotide of claim 1 or 2, wherein the multimerization domain or each multimerization domain comprises a lumazine synthase domain.

8. The circular polyribonucleotide of claim 1, wherein the open reading frame comprises, arranged in the following 5′-to-3′ order:
(a) a first sequence encoding an immunogen and second sequence encoding a T4 foldon domain;
(b) a first sequence encoding an immunogen and second sequence encoding a ferritin domain;
(c) a first sequence encoding an immunogen and second sequence encoding a β-annulus peptide;
(d) a first sequence encoding an immunogen and second sequence encoding an AaLS peptide;
(e) a first sequence encoding an immunogen, a second sequence encoding a T4 foldon domain, and a third sequence encoding an immunogen;
(f) a first sequence encoding an immunogen, a second sequence encoding a T4 foldon domain, and a third sequence encoding a ferritin domain; or
(g) a first sequence encoding an immunogen, a second sequence encoding a ferritin domain, and a third sequence encoding a T4 foldon domain.

9. The circular polyribonucleotide of any one of claims 1-8, wherein each immunogen is, independently, operably linked to a secretion signal sequence.

10. The circular polyribonucleotide of any one of claim 1-9, wherein the open reading frame is operably linked to an IRES.

11. The circular polyribonucleotide of any one of claims 1-10, wherein the circular polyribonucleotide further comprises a second open reading frame encoding a second polypeptide operably linked to a second IRES.

12. The circular polyribonucleotide of claim 11, wherein the second polypeptide is a polypeptide immunogen.

13. The circular polyribonucleotide of claim 11, wherein the second polypeptide is a polypeptide adjuvant.

14. The circular polyribonucleotide of claim 13, wherein the polypeptide adjuvant is a cytokine, a chemokine, a costimulatory molecule, an innate immune stimulator, a signaling molecule, a transcriptional activator, a cytokine receptor, a bacterial component, or a component of the innate immune system.

15. The circular polyribonucleotide of any one of claims 1-14, wherein the circular polyribonucleotide further comprises a non-coding ribonucleic acid sequence that is an innate immune system stimulator.

16. The circular polyribonucleotide of claim 15, wherein the innate immune system stimulator is selected from a GU-rich motif, an AU-rich motif, a structured region comprising dsRNA, or an aptamer.

17. An immunogenic composition comprising the circular polyribonucleotide of any one of claims 1-16 and a pharmaceutically acceptable excipient.

18. The immunogenic composition of claim 17, wherein the composition further comprises a second circular polyribonucleotide.

19. The immunogenic composition of claim 18, wherein the second circular polyribonucleotide comprises and open reading frame encoding an immunogen.

20. The immunogenic composition of claim 18, wherein the second circular polyribonucleotide comprises and open reading frame encoding a polypeptide adjuvant.

21. The immunogenic composition of claim 18, wherein the second circular polyribonucleotide comprises a non-coding ribonucleic acid sequence that is an innate immune system stimulator.

22. A method of inducing an immune response against an immunogen in a subject, the method comprising administering to the subject the circular polyribonucleotide or immunogenic composition of any one of claims 1-21.

23. A method of treating or preventing a disease, condition, or disorder in a subject, the method comprising administering to the subject the circular polyribonucleotide or immunogenic composition of any one of claims 1-21.

24. The method of claim 22 or 23, wherein the subject is a human subject.

25. The method of any one of claims 22-24, further comprising administering an adjuvant to the subject.

26. The method of any one of claims 22-25, further comprising administering a polypeptide immunogen to the subject.
